(19) United States
(12) Patent Application Publication
(10) Pub. No.: US 2009/0075255 A1
(43) Pub. Date: Mar. 19, 2009

(54) Androgen regulated nucleic acid molecules and encoded proteins

(75) Inventor: Biaoyang Lin, Bothell, WA (US)

Correspondence Address: MCDERMOTT, WILL & EMERY, 11682 EL CAMINO REAL, SUITE 400, SAN DIEGO, CA, 92130-2047, US

(73) Assignee: The Institute for Systems Biology, Seattle, WA (US)

(21) Appl. No.: 11/701,440

(22) Filed: Feb. 1, 2007

Related U.S. Application Data
(63) Continuation of application No. 10/345,837, filed on Jan. 15, 2003, now abandoned.
(60) Provisional application No. 60/505,349, filed on Jan. 15, 2002.

(51) Int. Cl. C12Q 1/68; C07H 21/04

(52) U.S. Cl. 435/6; 536/23.5

(57) ABSTRACT

The present invention provides novel androgen regulated nucleic acid molecules. Related polypeptides and diagnostic methods also are provided.

This application claims benefit of the filing date of U.S. Provisional Application No. 60/______ (yet to be assigned), filed Jan. 15, 2002, which was converted from U.S. Ser. No. 10/053,248, and which is incorporated herein by reference.

### BACKGROUND OF THE INVENTION

1. Field of the Invention

This invention relates generally to cancer and, more specifically, to prostate-specific genes that can be used to diagnose and treat prostate cancer.

2. Background Information

Cancer is currently the second leading cause of mortality in the United States. However, it is estimated that by the year 2000 cancer will surpass heart disease and become the leading cause of death in the United States. Prostate cancer is the most common non-cutaneous cancer in the United States and the second leading cause of male cancer mortality.

Cancerous tumors result when a cell escapes from its normal growth regulatory mechanisms and proliferates in an uncontrolled fashion. As a result of such uncontrolled proliferation, cancerous tumors usually invade neighboring tissues and spread by lymph or blood stream to create secondary or metastatic growths in other tissues. If untreated, cancerous tumors follow a fatal course. Prostate cancer, due to its slow growth profile, is an excellent candidate for early detection and therapeutic intervention.

During the last decade, most advances in prostate cancer research have focused on prostate specific antigen (PSA), a member of the serine protease family that exhibits a prostate-specific expression profile. Serum PSA remains the most widely used tumor marker for monitoring prostate cancer, but its specificity is limited by a high frequency of falsely elevated values in men with benign prostatic hyperplasia (BPH). Other biomarkers of prostate cancer progression have proven to be of limited clinical use in recent surveys because they are not uniformly elevated in men with advanced prostate cancer. Due to the limitations of currently available biomarkers, the identification and characterization of prostate specific genes is essential to the development of more accurate diagnostic methods and therapeutic targets. In many cases, the clinical potential of novel tumor markers can be optimized by utilizing them in combination with other tumor markers in the development of diagnostic and treatment modalities.

Normal prostate tissue consists of three distinct non-stromal cell populations, luminal secretory cells, basal cells, and endocrine paracrine cells. Phenotypic similarities between normal luminal cells and prostate cancer cells, including the expression of PSA, have suggested that prostate adenocarcinomas derive from luminal cells. However, a number of recent studies suggest that at least some prostate cancers can arise from the transformation of basal cells and report the expression of various genes in normal prostate basal cells as well as in prostate carcinoma cells. These genes include prostate stem cell antigen (PSCA), c-met and Bcl-2. Because none of these genes is universally expressed in all basal cells and prostate carcinomas, the utility of these genes as diagnostic markers is limited. Likewise, because PSA is expressed in luminal secretory cells in normal prostate tissue, this antigen has limited utility as a marker for basal cell derived carcinomas.

Thus, there exists a need for the identification of additional prostate specific genes that can be used as diagnostic markers and therapeutic targets for prostate cancer. The present invention satisfies this need and provides related advantages as well.

### SUMMARY OF THE INVENTION

The present invention provides androgen responsive prostate specific (ARP) nucleic acid and polypeptide molecules.

The present invention provides a substantially pure ARP7 nucleic acid molecule which includes the nucleotide sequence shown as SEQ ID NO: 1. The invention also provides a substantially pure ARP7 nucleic acid molecule that has at least 10 contiguous nucleotides of nucleotides 1-445 of SEQ ID NO: 1.

Further provided by the invention is method of diagnosing or predicting susceptibility to a prostate neoplastic condition in an individual. The method is practiced by contacting a sample from the individual with an ARP7 nucleic acid molecule containing at least 10 contiguous nucleotides of SEQ ID NO: 1; determining a test expression level of ARP7 RNA in the sample; and comparing the test expression level to a non-neoplastic control expression level of ARP7 RNA, where an altered test expression level as compared to the control expression level indicates the presence of a prostate neoplastic condition in the individual. In one embodiment, the method is practiced with a prostate tissue sample. In another embodiment, the method is practiced with a sample of blood, urine or semen. In a further embodiment, the method is practiced with an ARP7 nucleic acid molecule that has a length of 15 to 35 nucleotides.

The invention also provides a method of diagnosing or predicting susceptibility to a prostate neoplastic condition in an individual. The method includes the steps of contacting a specimen from the individual with an ARP7 binding agent that selectively binds an ARP7 polypeptide; determining a test expression level of ARP7 polypeptide in the specimen; and comparing the test expression level to a non-neoplastic control expression level of ARP7 polypeptide, where an altered test expression level as compared to the control expression level indicates the presence of a prostate neoplastic condition in the individual. A method of the invention can be practiced with a specimen that includes, for example, prostate tissue, or with a specimen which is blood, serum, urine or semen. If desired, a method of the invention for diagnosing or predicting susceptibility to a prostate neoplastic condition can be practiced with an ARP7 binding agent which is an antibody.

Also provided by the invention is a method for treating or reducing the severity of a prostate neoplastic condition in an individual by administering to the individual an ARP7 regulatory agent.

The present invention also provides a substantially pure ARP15 nucleic acid molecule that includes the nucleotide sequence shown as SEQ ID NO: 3. In addition, the invention provides a substantially pure ARP15 nucleic acid molecule which has at least 10 contiguous nucleotides of nucleotides 1-86 of SEQ ID NO: 3.

Also provided herein is a method of diagnosing or predicting susceptibility to a prostate neoplastic condition in an individual by contacting a sample from the individual with an ARP15 nucleic acid molecule that includes at least 10 contiguous nucleotides of SEQ ID NO: 3; determining a test expression level of ARP15 RNA in the sample; and comparing the test expression level to a non-neoplastic control expression level of ARP15 RNA, where an altered test expression level as compared to the control expression level indicates the presence of a prostate neoplastic condition in the individual. A sample useful in such a method of the invention can include, for example, prostate tissue, or can be, for example, blood, urine or semen. An ARP15 nucleic acid molecule useful in a method of the invention can have a length of, for example, 15 to 35 nucleotides.

The invention also provides a method of diagnosing or predicting susceptibility to a prostate neoplastic condition in an individual by contacting a specimen from the individual with an ARP15 binding agent that selectively binds an ARP15 polypeptide; determining a test expression level of ARP15 polypeptide in the specimen; and comparing the test expression level to a non-neoplastic control expression level of ARP15 polypeptide, where an altered test expression level as compared to the control expression level indicates the presence of a prostate neoplastic condition in the individual. A specimen useful in such a method can include, for example, prostate tissue, or can be, for example, blood, serum, urine or semen. In one embodiment, the ARP15 binding agent that selectively binds the ARP15 polypeptide is an antibody.

Further provided herein is a method for treating or reducing the severity of a prostate neoplastic condition in an individual by administering to the individual an ARP15 regulatory agent.

The present invention additionally provides a substantially pure ARP16 nucleic acid molecule that contains a nucleic acid sequence encoding an ARP16 polypeptide having at least 90% amino acid identity with SEQ ID NO: 6. Such a nucleic acid molecule can encode, for example, the amino acid sequence shown as SEQ ID NO:6. In one embodiment, an ARP16 nucleic acid molecule of the invention includes the nucleotide sequence shown as SEQ ID NO:5. Further provided by the invention is a substantially pure ARP16 nucleic acid molecule that includes at least 10 contiguous nucleotides of nucleotides 1-1531 of SEQ ID NO: 5.

The invention also provides a method of diagnosing or predicting susceptibility to a prostate neoplastic condition in an individual by contacting a sample from the individual with an ARP16 nucleic acid molecule containing at least 10 contiguous nucleotides of SEQ ID NO: 5; determining a test expression level of ARP16 RNA in the sample; and comparing the test expression level to a non-neoplastic control expression level of ARP16 RNA, where an altered test expression level as compared to the control expression level indicates the presence of a prostate neoplastic condition in the individual. Samples useful in the methods of the invention include, for example, prostate tissue samples as well as samples of blood, urine or semen. In one embodiment, a method of the invention is practiced with an ARP16 nucleic acid molecule which has a length of 15 to 35 nucleotides.

The invention also provides a substantially pure ARP16 polypeptide that contains an amino acid sequence having at least 90% amino acid identity with SEQ ID NO: 6. An ARP16 polypeptide of the invention can include, for example, the amino acid sequence shown as SEQ ID NO: 6.

Also provided by the invention is a substantially pure ARP16 polypeptide fragment which has at least eight contiguous amino acids of SEQ ID NO: 6. In one embodiment, an ARP16 polypeptide fragment of the invention has at least eight contiguous amino acids of residues 1-465 of SEQ ID NO: 6.

Also provided herein is an ARP16 binding agent which includes a molecule that selectively binds at least eight contiguous amino acids of SEQ ID NO: 6. In one embodiment, such a binding agent selectively binds at least eight contiguous amino acids of residues 1-465 of SEQ ID NO: 6. In another embodiment, the ARP16 binding agent is an antibody.

Also provided herein is a method of diagnosing or predicting susceptibility to a prostate neoplastic condition in an individual by contacting a specimen from the individual with an ARP16 binding agent that selectively binds an ARP16 polypeptide; determining a test expression level of ARP16 polypeptide in the specimen; and comparing the test expression level to a non-neoplastic control expression level of ARP16 polypeptide, where an altered test expression level as compared to the control expression level indicates the presence of a prostate neoplastic condition in the individual. A specimen useful for diagnosing or predicting susceptibility to a prostate neoplastic condition can include, for example, prostate tissue, or can be, for example, a specimen of blood, serum, urine or semen. In one embodiment, the ARP16 binding agent that selectively binds the ARP16 polypeptide is a antibody.

Further provided herein is a method for treating or reducing the severity of a prostate neoplastic condition in an individual by administering to the individual an ARP16 regulatory agent.

The present invention also provides a substantially pure ARP8 nucleic acid molecule that contains a nucleic acid sequence encoding an ARP8 polypeptide having at least 65% amino acid identity with SEQ ID NO: 8. Such a substantially pure ARP8 nucleic acid molecule can encode, for example, the amino acid sequence shown as SEQ ID NO: 8. In one embodiment, an ARP8 nucleic acid molecule of the invention has the nucleotide sequence shown as SEQ ID NO: 7. Also provided herein is a substantially pure ARP8 nucleic acid molecule which includes at least 10 contiguous nucleotides of nucleotides 1-349 of SEQ ID NO: 7.

The invention additionally provides method of diagnosing or predicting susceptibility to a prostate neoplastic condition in an individual. The method includes the steps of contacting a sample from the individual with an ARP8 nucleic acid molecule containing at least 10 contiguous nucleotides of SEQ ID NO:7; determining a test expression level of ARP8 RNA in the sample; and comparing the test expression level to a non-neoplastic control expression level of ARP8 RNA, where an altered test expression level as compared to the control expression level indicates the presence of a prostate neoplastic condition in the individual. In one embodiment, the sample includes prostate tissue. In other embodiments, the sample is blood, urine or semen. In a further embodiment, the ARP8 nucleic acid molecule has a length of 15 to 35 nucleotides.

The present invention further provides a substantially pure ARP8 polypeptide that contains an amino acid sequence having at least 65% amino acid identity with SEQ ID NO: 8. Such an ARP8 polypeptide can have, for example, the amino acid sequence shown as SEQ ID NO: 8. In addition, there is provided herein a substantially pure ARP8 polypeptide fragment, which includes at least eight contiguous amino acids of residues 1-116 of SEQ ID NO: 8. In one embodiment, the ARP8 fragment has at least eight contiguous amino acids of residues 249-576 of SEQ ID NO: 8.

Also provided herein is an ARP8 binding agent which includes a molecule that selectively binds at least eight contiguous amino acids of residues 1-116 of SEQ ID NO: 8, for example, an antibody that selectively binds at least eight contiguous amino acids of residues 1-116 of SEQ ID NO: 8. In addition, the invention provides an ARP8 binding agent which includes a molecule that selectively binds at least eight contiguous amino acids of residues 249-576 of SEQ ID NO: 8. Such an ARP8 binding agent can be, for example, an antibody.

There is further provided herein a method of diagnosing or predicting susceptibility to a prostate neoplastic condition in an individual by contacting a specimen from the individual with an ARP8 binding agent that selectively binds an ARP8 polypeptide; determining a test expression level of ARP8 polypeptide in the specimen; and comparing the test expression level to a non-neoplastic control expression level of ARP8 polypeptide, where an altered test expression level as compared to the control expression level indicates the presence of a prostate neoplastic condition in the individual. A method of the invention can be practiced, for example, with a specimen that includes prostate tissue, or with a specimen which is blood, serum, urine or semen. In one embodiment, the ARP8 binding agent that selectively binds the ARP8 polypeptide is an antibody.

Also provided herein is a method for treating or reducing the severity of a prostate neoplastic condition in an individual by administering to the individual an ARP8 regulatory agent.

The present invention further provides a substantially pure ARP9 nucleic acid molecule that includes a nucleic acid sequence encoding an ARP9 polypeptide having at least 65% amino acid identity with SEQ ID NO: 10. A substantially pure ARP9 nucleic acid molecule of the invention can encode, for example, the amino acid sequence shown as SEQ ID NO: 10. In one embodiment, the nucleic acid molecule includes the nucleotide sequence shown as SEQ ID NO:9. The invention also provides a substantially pure ARP9 nucleic acid molecule that includes at least 10 contiguous nucleotides of nucleotides 697-745 of SEQ ID NO: 9.

Further provided herein is a method of diagnosing or predicting susceptibility to a prostate neoplastic condition in an individual. The method is practiced by contacting a sample from the individual with an ARP9 nucleic acid molecule that includes at least 10 contiguous nucleotides of SEQ ID NO: 9; determining a test expression level of ARP9 RNA in the sample; and comparing the test expression level to a non-neoplastic control expression level of ARP9 RNA, where an altered test expression level as compared to the control expression level indicates the presence of a prostate neoplastic condition in the individual. In one embodiment, a method of the invention is practiced with a sample that includes prostate tissue. In other embodiments, a method of the invention is practiced with a sample of blood, urine or semen. In a further embodiment, a method of the invention is practiced with an ARP9 nucleic acid molecule having a length of 15 to 35 nucleotides.

The invention also provides a substantially pure ARP9 polypeptide that includes an amino acid sequence having at least 65% amino acid identity with SEQ ID NO: 10. Such an ARP9 polypeptide can have, for example, the amino acid sequence shown as SEQ ID NO: 10. Substantially pure ARP9 polypeptide fragments also are provided herein. The ARP9 fragments of the invention have at least eight contiguous amino acids of residues 1-83 of SEQ ID NO: 10. In one embodiment, such an ARP9 fragment of the invention has at least eight contiguous amino acids of residues 47-62 of SEQ ID NO: 10.

The invention also provides an ARP9 binding agent that includes a molecule that selectively binds at least eight contiguous amino acids of residues 1-83 of SEQ ID NO: 10. In one embodiment, the ARP9 binding agent includes a molecule that selectively binds at least eight contiguous amino acids of residues 47-62 of SEQ ID NO: 10. An ARP9 binding agent of the invention can be, for example, an antibody.

The present invention further provides a method of diagnosing or predicting susceptibility to a prostate neoplastic condition in an individual, in which a specimen from the individual is contacted with an ARP9 binding agent that selectively binds an ARP9 polypeptide; a test expression level of ARP9 polypeptide in the specimen is determined; and the test expression level is compared to a non-neoplastic control expression level of ARP9 polypeptide, where an altered test expression level as compared to the control expression level indicates the presence of a prostate neoplastic condition in the individual. A method of the invention can be practiced with a specimen containing, for example, prostate tissue, or, for example, with a blood, serum, urine or semen specimen. If desired, a method of the invention can be practiced with an ARP9 binding agent which is an antibody.

Further provided herein is a method for treating or reducing the severity of a prostate neoplastic condition in an individual by administering to the individual an ARP9 regulatory agent.

The present invention also provides a substantially pure ARP13 nucleic acid molecule that includes a nucleic acid sequence encoding an ARP13 polypeptide having at least 90% amino acid identity with SEQ ID NO: 12. Such a substantially pure ARP13 nucleic acid molecule can encode, for example, the amino acid sequence shown as SEQ ID NO: 12. In one embodiment, a substantially pure ARP13 nucleic acid molecule of the invention has the nucleotide sequence shown as SEQ ID NO: 11.

The invention also provides a method of diagnosing or predicting susceptibility to a prostate neoplastic condition in an individual. The method includes the steps of contacting a sample from the individual with an ARP13 nucleic acid molecule that includes at least 10 contiguous nucleotides of SEQ ID NO: 11; determining a test expression level of ARP13 RNA in the sample; and comparing the test expression level to a non-neoplastic control expression level of ARP13 RNA, where an altered test expression level as compared to the control expression level indicates the presence of a prostate neoplastic condition in the individual. A method of the invention can be practiced, for example, with a sample which includes prostate tissue or, for example, with a blood, urine or semen sample. A variety of ARP13 nucleic acid molecules are useful in the methods of the invention including ARP13 nucleic acid molecules of 15 to 35 nucleotides in length.

Also provided herein is a substantially pure ARP13 polypeptide, which has an amino acid sequence having at least 90% amino acid identity with SEQ ID NO: 12. As an example, a substantially pure ARP13 polypeptide of the invention can have the amino acid sequence shown as SEQ ID NO: 12. The invention additionally provides a substantially pure ARP13 polypeptide fragment that includes at least eight contiguous amino acids of SEQ ID NO: 12.

There further is provided herein an ARP13 binding agent which includes a molecule that selectively binds at least eight contiguous amino acids of SEQ ID NO: 12. In one embodiment, the ARP13 binding agent is an antibody.

The invention also provides a method of diagnosing or predicting susceptibility to a prostate neoplastic condition in an individual by contacting a specimen from the individual with an ARP13 binding agent that selectively binds an ARP13 polypeptide; determining a test expression level of ARP13 polypeptide in the specimen; and comparing the test expression level to a non-neoplastic control expression level of ARP13 polypeptide, where an altered test expression level as compared to the control expression level indicates the presence of a prostate neoplastic condition in the individual. A variety of specimens are useful in a method of the invention for diagnosing or predicting susceptibility to a prostate neoplastic condition, including, but not limited to, prostate tissue, blood, serum, urine and semen. An ARP13 binding agent useful in a method of the invention can be, for example, an antibody.

Further provided herein is a method for treating or reducing the severity of a prostate neoplastic condition in an individual by administering to the individual an ARP13 regulatory agent.

There further is provided herein a method of diagnosing or predicting susceptibility to a prostate neoplastic condition in an individual by contacting a sample from the individual with an ARP20 nucleic acid molecule which includes at least 10 contiguous nucleotides of SEQ ID NO: 13; determining a test expression level of ARP20 RNA in the sample; and comparing the test expression level to a non-neoplastic control expression level of ARP20 RNA, where an altered test expression level as compared to the control expression level indicates the presence of a prostate neoplastic condition in the individual. Samples useful in a method of the invention include prostate tissue, blood, urine and semen. In one embodiment, a method of the invention is practiced with an ARP20 nucleic acid molecule having a length of 15 to 35 nucleotides.

The invention also provides a substantially pure ARP20 polypeptide that includes an amino acid sequence having at least 55% amino acid identity with SEQ ID NO: 14. Such an ARP20 polypeptide can have, for example, the amino acid sequence shown as SEQ ID NO: 14. Also provided herein is a substantially pure ARP20 polypeptide fragment including at least eight contiguous amino acids of SEQ ID NO: 14.

The invention also provides an ARP20 binding agent which contains a molecule that selectively binds at least eight contiguous amino acids of SEQ ID NO: 14. In one embodiment, the ARP20 binding agent is an antibody.

Further provided herein is a method of diagnosing or predicting susceptibility to a prostate neoplastic condition in an individual. The method is practiced by contacting a specimen from the individual with an ARP20 binding agent that selectively binds an ARP20 polypeptide; determining a test expression level of ARP20 polypeptide in the specimen; and comparing the test expression level to a non-neoplastic control expression level of ARP20 polypeptide, where an altered test expression level as compared to the control expression level indicates the presence of a prostate neoplastic condition in the individual. In one embodiment, a method of the invention is practiced with a specimen of prostate tissue. In another embodiment, a method of the invention is practiced with a blood, serum, urine or semen specimen. In a further embodiment, a method of the invention is practiced with an ARP20 binding agent which is an antibody.

The invention further provides a method for treating or reducing the severity of a prostate neoplastic condition in an individual by administering to the individual an ARP20 regulatory agent.

Also provided herein is a method of diagnosing or predicting susceptibility to a prostate neoplastic condition in an individual. The method includes the steps of contacting a sample from the individual with an ARP24 nucleic acid molecule containing at least 10 contiguous nucleotides of SEQ ID NO: 15; determining a test expression level of ARP24 RNA in the sample; and comparing the test expression level to a non-neoplastic control expression level of ARP24 RNA, where an altered test expression level as compared to the control expression level indicates the presence of a prostate neoplastic condition in the individual. In one embodiment, a method of the invention is practiced with a sample containing prostate tissue. In other embodiments, a method of the invention is practiced with a sample of blood, urine or semen. In yet another embodiment, the method is practiced with an ARP24 nucleic acid molecule that is 15 to 35 nucleotides in length.

Further provided herein is a substantially pure ARP24 polypeptide that includes an amino acid sequence having at least 30% amino acid identity with SEQ ID NO: 16. A substantially pure ARP24 polypeptide of the invention can have, for example, the amino acid sequence shown as SEQ ID NO: 16. The invention also provides a substantially pure ARP24 polypeptide fragment which contains at least eight contiguous amino acids of SEQ ID NO: 16.

In addition, there is provided herein an ARP24 binding agent that includes a molecule that selectively binds at least eight contiguous amino acids of SEQ ID NO: 16. In one embodiment, the ARP24 binding agent is an antibody.

The invention also provides a method of diagnosing or predicting susceptibility to a prostate neoplastic condition in an individual by contacting a specimen from the individual with an ARP24 binding agent that selectively binds an ARP24 polypeptide; determining a test expression level of ARP24 polypeptide in the specimen; and comparing the test expression level to a non-neoplastic control expression level of ARP24 polypeptide, where an altered test expression level as compared to the control expression level indicates the presence of a prostate neoplastic condition in the individual. Samples useful in a method of the invention include prostate tissue, blood, urine and semen. In one embodiment, a method of the invention is practiced with an ARP24 nucleic acid molecule having a length of 15 to 35 nucleotides.

Further provided herein is a method for treating or reducing the severity of a prostate neoplastic condition in an individual by administering to the individual an ARP24 regulatory agent.

The present invention further provides a substantially pure ARP26 nucleic acid which includes the nucleotide sequence shown as SEQ ID NO: 17. The invention also provides a substantially pure ARP26 nucleic acid molecule of the invention that includes at least 10 contiguous nucleotides of nucleotides 1404-1516 of SEQ ID NO: 17.

Also provided herein is a method of diagnosing or predicting susceptibility to a prostate neoplastic condition in an individual. A method of the invention includes the steps of contacting a sample from the individual with an ARP26 nucleic acid molecule containing at least 10 contiguous nucleotides of SEQ ID NO: 17; determining a test expression level of ARP26 RNA in the sample; and comparing the test expression level to a non-neoplastic control expression level of ARP26 RNA, where an altered test expression level as compared to the control expression level indicates the presence of a prostate neoplastic condition in the individual. Samples useful in a method of the invention include prostate tissue, blood, urine and semen. In one embodiment, a method of the invention is practiced with an ARP26 nucleic acid molecule having a length of 15 to 35 nucleotides.

The invention also provides a method of diagnosing or predicting susceptibility to a prostate neoplastic condition in an individual by contacting a specimen from the individual with an ARP26 binding agent that selectively binds an ARP26 polypeptide; determining a test expression level of ARP26 polypeptide in the specimen; and comparing the test expression level to a non-neoplastic control expression level of ARP26 polypeptide, where an altered test expression level as compared to the control expression level indicates the presence of a prostate neoplastic condition in the individual. A specimen useful in the invention can include, for example, prostate tissue, or can be, for example, a blood, serum, urine or semen specimen. In one embodiment, the ARP26 binding agent is an antibody.

The invention also provides a method for treating or reducing the severity of a prostate neoplastic condition in an individual by administering to the individual an ARP26 regulatory agent.

The invention further provides a method of diagnosing or predicting susceptibility to a prostate neoplastic condition in an individual, in which a sample from the individual is contacted with an ARP28 nucleic acid molecule containing at least 10 contiguous nucleotides of SEQ ID NO: 19; a test expression level of ARP28 RNA in the sample is determined; and the test expression level is compared to a non-neoplastic control expression level of ARP28 RNA, where an altered test expression level as compared to the control expression level indicates the presence of a prostate neoplastic condition in the individual. In one embodiment, the sample contacted with an ARP28 nucleic acid molecule contains prostate tissue. In other embodiments, the sample is blood, urine or semen sample. In a further embodiment, the ARP28 nucleic acid molecule has a length of 15 to 35 nucleotides.

The invention further provides herein a method of diagnosing or predicting susceptibility to a prostate neoplastic condition in an individual by contacting a specimen from the individual with an ARP28 binding agent the selectively binds an ARP28 polypeptide; determining a test expression level of ARP28 polypeptide in the specimen; and comparing the test expression level to a non-neoplastic control expression level of ARP28 polypeptide, where an altered test expression level as compared to the control expression level indicates the presence of a prostate neoplastic condition in the individual. A specimen useful in the invention can include, for example, prostate tissue, or can be, for example, a blood, serum, urine or semen specimen. ARP28 binding agents useful in the methods of the invention include, but are not limited to, antibodies.

The invention further provides a method for treating or reducing the severity of a prostate neoplastic condition in an individual by administering to the individual an ARP28 regulatory agent.

The present invention also provides a substantially pure ARP30 nucleic acid molecule that includes a nucleic acid sequence encoding an ARP30 polypeptide having at least 30% amino acid identity with SEQ ID NO: 22. A substantially pure ARP30 nucleic acid molecule of the invention can encode, for example, the amino acid sequence shown as SEQ ID NO: 22, and, in one embodiment, includes the nucleotide sequence shown as SEQ ID NO: 21. Also provided herein is a substantially pure ARP30 nucleic acid molecule that includes at least 10 contiguous nucleotides of nucleotides 1-132, nucleotides 832-1696, or nucleotides 2346-2796 of SEQ ID NO: 21.

The invention also provides herein a method of diagnosing or predicting susceptibility to a prostate neoplastic condition in an individual. This method includes the steps of contacting a sample from the individual with an ARP30 nucleic acid molecule containing at least 10 contiguous nucleotides of nucleotides 1-1829 or nucleotides 2346-3318 of SEQ ID NO: 21; determining a test expression level of ARP30 RNA in the sample; and comparing the test expression level to a non-neoplastic control expression level of ARP30 RNA, where an altered test expression level as compared to the control expression level indicates the presence of a prostate neoplastic condition in the individual. In one embodiment, a method of the invention is practiced with a sample containing prostate tissue. In other embodiments, a method of the invention is practiced with a blood, urine or semen sample. In a further embodiment, a method of the invention is practiced with an ARP30 nucleic acid molecule having a length of 15 to 35 nucleotides.

Also provided herein is a substantially pure ARP30 polypeptide that contains an amino acid sequence having at least 30% amino acid identity with SEQ ID NO: 22. In one embodiment, a substantially pure ARP30 polypeptide of the invention encodes the amino acid sequence shown as SEQ ID NO: 22. The invention also provides a substantially pure ARP30 polypeptide fragment that has at least eight contiguous amino acids of SEQ ID NO: 22.

In addition, there is provided herein an ARP30 binding agent, which includes a molecule that selectively binds at least eight contiguous residues of SEQ ID NO: 22. In one embodiment, the ARP30 binding agent is an antibody.

The invention also provides herein a method of diagnosing or predicting susceptibility to a prostate neoplastic condition in an individual by contacting a specimen from the individual with an ARP30 binding agent that selectively binds an ARP30 polypeptide; determining a test expression level of ARP30 polypeptide in the specimen; and comparing the test expression level to a non-neoplastic control expression level of ARP30 polypeptide, where an altered test expression level as compared to the control expression level indicates the presence of a prostate neoplastic condition in the individual. A specimen useful in the invention can include, for example, prostate tissue, or can be, for example, a blood, serum, urine or semen specimen. ARP30 binding agents useful in the methods of the invention include, but are not limited to, antibodies.

The invention further provides a method for treating or reducing the severity of a prostate neoplastic condition in an individual by administering to the individual an ARP30 regulatory agent.

The invention also provides a method of diagnosing or predicting susceptibility to a prostate neoplastic condition in an individual by contacting a sample from the individual with an ARP33 nucleic acid molecule that includes at least 10 contiguous nucleotides of SEQ ID NO: 23; determining a test expression level of ARP33 RNA in the sample; and comparing the test expression level to a non-neoplastic control expression level of ARP33 RNA, where an altered test expression level as compared to the control expression level indicates the presence of a prostate neoplastic condition in the individual. Samples useful in the invention can include, for example, prostate tissue. Samples useful in the invention also can be samples of blood, urine or semen. A variety of ARP33 nucleic acid molecules are useful in the methods of the invention including, for example, ARP33 nucleic acid molecules of 15 to 35 nucleotides in length.

The invention also provides a substantially pure ARP33 polypeptide that includes an amino acid sequence having at least 70% amino acid identity with SEQ ID NO: 24. Such a substantially pure ARP33 polypeptide can have, for example, the amino acid sequence shown as SEQ ID NO: 24. Also provided herein is a substantially pure ARP33 polypeptide fragment that includes at least eight contiguous amino acids of residues 1-132 or 251-405 of SEQ ID NO: 24.

The present invention also provides an ARP33 binding agent that includes a molecule that selectively binds at least eight contiguous amino acids of residues 1-132 or 251-405 of SEQ ID NO: 24. Such an ARP33 binding agent can be, for example, an antibody.

The invention also provides herein a method of diagnosing or predicting susceptibility to a prostate neoplastic condition in an individual by contacting a specimen from the individual with an ARP33 binding agent that selectively binds an ARP33 polypeptide; determining a test expression level of ARP33 polypeptide in the specimen; and comparing the test expression level to a non-neoplastic control expression level of ARP33 polypeptide, where an altered test expression level as compared to the control expression level indicates the presence of a prostate neoplastic condition in the individual. A specimen useful in the invention can include, for example, prostate tissue, or can be, for example, a blood, serum, urine or semen specimen. ARP33 binding agents useful in the methods of the invention encompass, without limitation, antibodies.

The invention further provides herein a method for treating or reducing the severity of a prostate neoplastic condition in an individual by administering to the individual an ARP33 regulatory agent.

The present invention also provides a substantially pure ARP11 nucleic acid molecule that contains the nucleotide sequence shown as SEQ ID NO: 33. In addition, there is provided a substantially pure ARP11 nucleic acid molecule which contains at least 10 contiguous nucleotides of nucleotides 1-458 of SEQ ID NO: 33.

Also provided herein is a method of diagnosing or predicting susceptibility to a prostate neoplastic condition in an individual by contacting a sample from the individual with an ARP11 nucleic acid molecule containing at least 10 contiguous nucleotides of nucleotides 1-458 of SEQ ID NO: 33; determining a test expression level of ARP11 RNA in the sample; and comparing the test expression level to a non-neoplastic control expression level of ARP11 RNA, where an altered test expression level as compared to the control expression level indicates the presence of a prostate neoplastic condition in the individual. A sample useful for diagnosing or predicting susceptibility to a prostate neoplastic condition according to a method of the invention can be, for example, a sample of prostate tissue or a sample of blood, urine or semen. In one embodiment, a method of the invention is practiced with an ARP11 nucleic acid molecule having a length of 15 to 35 nucleotides.

The invention further provides a substantially pure ARP11 polypeptide which contains an amino acid sequence having at least 75% amino acid identity with SEQ ID NO: 34. Such an ARP11 polypeptide can include, for example, the amino acid sequence shown as SEQ ID NO: 34. Also provided is a substantially pure ARP11 polypeptide fragment containing at least eight contiguous amino acids of SEQ ID NO: 34.

Further provided herein is an ARP11 binding agent that contains a molecule which selectively binds at least eight contiguous amino acids of SEQ ID NO: 34. Such a binding agent can be, for example, an antibody.

The present invention also provides a method of diagnosing or predicting susceptibility to a prostate neoplastic condition in an individual by contacting a specimen from the individual with an ARP11 binding agent that selectively binds an ARP11 polypeptide; determining a test expression level of ARP11 polypeptide in the specimen; and comparing the test expression level to a non-neoplastic control expression level of ARP11 polypeptide, where an altered test expression level as compared to the control expression level indicates the presence of a prostate neoplastic condition in the individual. The method can be practiced with, for example, a prostate tissue specimen, or with a specimen of blood, serum, urine or semen. In one embodiment, a method of the invention is practiced with an ARP11 binding agent which is an antibody.

The invention further provides a method for treating or reducing the severity of a prostate neoplastic condition in an individual by administering to the individual an ARP11 regulatory agent.

The invention also provides a substantially pure ARP6 nucleic acid molecule that includes the nucleotide sequence shown as SEQ ID NO: 25. Further provided herein is a substantially pure ARP6 nucleic acid molecule that contains at least 10 contiguous nucleotides of nucleotides 505-526 of SEQ ID NO: 25.

The invention additionally provides a method of diagnosing or predicting susceptibility to a prostate neoplastic condition in an individual by contacting a sample from the individual with an ARP6 nucleic acid molecule containing at least 10 contiguous nucleotides of SEQ ID NO: 25; determining a test expression level of ARP6 RNA in the sample; and comparing the test expression level to a non-neoplastic control expression level of ARP6 RNA, where an altered test expression level as compared to the control expression level indicates the presence of a prostate neoplastic condition in the individual. In one embodiment, the method is practiced with a prostate tissue sample. In another embodiment, the method is practiced with a sample of blood, urine or semen. In a further embodiment, the method is practiced with an ARP6 nucleic acid molecule having a length of 15 to 35 nucleotides.

The invention further provides a method for treating or reducing the severity of a prostate neoplastic condition in an individual by administering to the individual an ARP6 regulatory agent.

The invention further provides a method of diagnosing or predicting susceptibility to a prostate neoplastic condition in an individual by contacting a sample from the individual with an ARP11 nucleic acid molecule containing at least 10 contiguous nucleotides of SEQ ID NO: 26; determining a test expression level of ARP11 RNA in the sample; and comparing the test expression level to a non-neoplastic control expression level of ARP11 RNA, where an altered test expression level as compared to the control expression level indicates the presence of a prostate neoplastic condition in the individual. In one embodiment, the method is practiced with a sample containing prostate tissue. In other embodiments, the method is practiced with a blood, urine or semen sample. In a further embodiment, the method is practiced with an ARP11 nucleic acid molecule of 15 to 35 nucleotides in length.

The invention further provides a method for treating or reducing the severity of a prostate neoplastic condition in an individual by administering to the individual an ARP11 regulatory agent.

The present invention further provides a substantially pure ARP12 nucleic acid molecule that contains the nucleotide sequence shown as SEQ ID NO: 27. In addition, the invention provides a substantially pure ARP12 nucleic acid molecule that contains at least 10 contiguous nucleotides of nucleotides 1635-1659 of SEQ ID NO: 27.

Also provided herein is a method of diagnosing or predicting susceptibility to a prostate neoplastic condition in an individual. This method includes the steps of contacting a sample from the individual with an ARP12 nucleic acid molecule containing at least 10 contiguous nucleotides of nucleotides 1-1659 or 2176-2576 of SEQ ID NO: 27; determining a test expression level of ARP12 RNA in the sample; and comparing the test expression level to a non-neoplastic control expression level of ARP12 RNA, where an altered test expression level as compared to the control expression level indicates the presence of a prostate neoplastic condition in the individual. In one embodiment, the method is practiced with a sample containing prostate tissue. In other embodiments, the method is practiced with a blood, urine or semen sample. In a further embodiment, a method of the invention is practiced with an ARP12 nucleic acid molecule that has a length of 15 to 35 nucleotides.

There further is provided herein a method for treating or reducing the severity of a prostate neoplastic condition in an individual by administering to the individual an ARP12 regulatory agent.

The present invention additionally provides a method of diagnosing or predicting susceptibility to a prostate neoplastic condition in an individual by contacting a sample from the individual with an ARP18 nucleic acid molecule containing at least 10 contiguous nucleotides of SEQ ID NO: 28; determining a test expression level of ARP18 RNA in the sample; and comparing the test expression level to a non-neoplastic control expression level of ARP18 RNA, where an altered test expression level as compared to the control expression level indicates the presence of a prostate neoplastic condition in the individual. A method of the invention can be practiced, for example, with a sample containing prostate tissue, or, for example, with a sample of blood, urine or semen. A variety of ARP18 nucleic acid molecules are useful in the methods of the invention. In one embodiment, the invention is practiced with an ARP18 nucleic acid molecule which has a length of 15 to 35 nucleotides.

The invention also provides a method for treating or reducing the severity of a prostate neoplastic condition in an individual by administering to the individual an ARP18 regulatory agent.

The invention also provided herein a substantially pure ARP19 nucleic acid molecule that includes the nucleotide sequence shown as SEQ ID NO: 29. Furthermore, there is provided herein a substantially pure ARP19 nucleic acid molecule which has at least 10 contiguous nucleotides of nucleotides 1-31 and 478-644 of SEQ ID NO: 29.

The invention further provides a method of diagnosing or predicting susceptibility to a prostate neoplastic condition in an individual by contacting a sample from the individual with an ARP19 nucleic acid molecule containing at least 10 contiguous nucleotides of SEQ ID NO: 29; determining a test expression level of ARP19 RNA in the sample; and comparing the test expression level to a non-neoplastic control expression level of ARP19 RNA, where an altered test expression level as compared to the control expression level indicates the presence of a prostate neoplastic condition in the individual. A method of the invention can be practiced, for example, with a sample containing prostate tissue, or, for example, with a sample of blood, urine or semen. A variety of ARP19 nucleic acid molecules are useful in the methods of the invention, for example, ARP19 nucleic acid molecules of 15 to 35 nucleotides in length.

The invention further provides a method for treating or reducing the severity of a prostate neoplastic condition in an individual by administering to the individual an ARP19 regulatory agent.

The present invention also provides a method of diagnosing or predicting susceptibility to a prostate neoplastic condition in an individual by contacting a sample from the individual with an ARP21 nucleic acid molecule containing at least 10 contiguous nucleotides of SEQ ID NO: 30; determining a test expression level of ARP21 RNA in the sample; and comparing the test expression level to a non-neoplastic control expression level of ARP21 RNA, where an altered test expression level as compared to the control expression level indicates the presence of a prostate neoplastic condition in the individual. Samples useful in the invention include, without limitation, those containing prostate tissue as well as blood, urine and semen samples. In one embodiment, a method of the invention is practiced with an ARP21 nucleic acid molecule having a length of 15 to 35 nucleotides.

The present invention also provides a method for treating or reducing the severity of a prostate neoplastic condition in an individual by administering to the individual an ARP21 regulatory agent.

The present invention also provides a substantially pure ARP22 nucleic acid molecule which includes the nucleotide sequence shown as SEQ ID NO: 31. In addition, the invention provides a substantially pure ARP22 nucleic acid molecule that has at least 10 contiguous nucleotides of nucleotides 1-73 or 447-464 of SEQ ID NO: 31.

Further provided by the present invention is a method of diagnosing or predicting susceptibility to a prostate neoplastic condition in an individual by contacting a sample from the individual with an ARP22 nucleic acid molecule containing at least 10 contiguous nucleotides of SEQ ID NO: 31; determining a test expression level of ARP22 RNA in the sample; and comparing the test expression level to a non-neoplastic control expression level of ARP22 RNA, where an altered test expression level as compared to the control expression level indicates the presence of a prostate neoplastic condition in the individual. In one embodiment, the method is practiced with a sample containing prostate tissue. In other embodiments, the method is practiced with a blood, urine or semen sample. In a further embodiment, a method of the invention is practiced with an ARP22 nucleic acid molecule having a length of 15 to 35 nucleotides.

The present invention also provides a method for treating or reducing the severity of a prostate neoplastic condition in an individual by administering to the individual an ARP22 regulatory agent.

The present invention also provides a method of diagnosing or predicting susceptibility to a prostate neoplastic condition in an individual by contacting a sample from the individual with an ARP29 nucleic acid molecule containing at least 10 contiguous nucleotides of SEQ ID NO: 32; determining a test expression level of ARP29 RNA in the sample; and comparing the test expression level to a non-neoplastic control expression level of ARP29 RNA, where an altered test expression level as compared to the control expression level indicates the presence of a prostate neoplastic condition in the individual. In one embodiment, the method is practiced with a sample containing prostate tissue. In other embodiments, the method is practiced with a sample of blood, urine or semen. In a further embodiment, a method of the invention is practiced with an ARP29 nucleic acid molecule which has a length of 15 to 35 nucleotides.

In addition, there is provided herein a method for treating or reducing the severity of a prostate neoplastic condition in an individual by administering to the individual an ARP29 regulatory agent.

### BRIEF DESCRIPTION OF THE DRAWINGS

FIG. 1 shows northern analysis of ARP7, ARp15, ARP16 and ARP21 expression in androgen stimulated cells. “+” indicates androgen-stimulated RNA; “−” indicates androgen-starved RNA.

FIG. 2 shows hybridization of an ARP7 probe to two multiple tissue northern blots (Clontech).

FIG. 3 shows hybridization of an ARP15 probe to two multiple tissue northern blots (Clontech).

FIG. 4 shows hybridization of an ARP21 probe to two multiple tissue northern blots (Clontech).

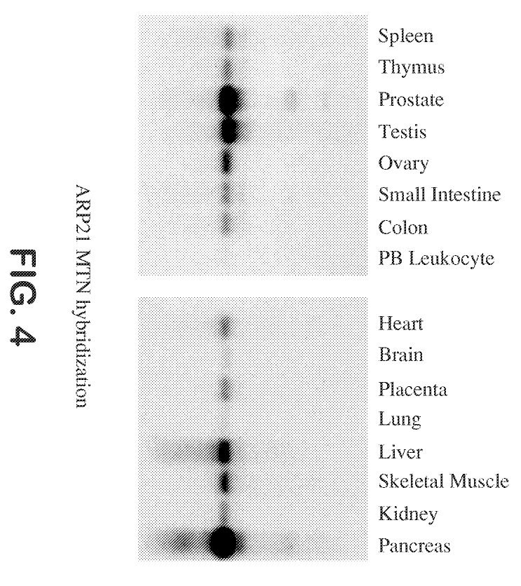

FIG. 5 shows Western blot analysis of ARP15 protein in cell lysates from prostate cancer LNCaP cells (left lane: “LNCaP”) and in serum from a prostate cancer patient (right lane: “Cap Serum”).

FIG. 6 shows cellular localization of ARP15. (A) LNCaP cells stained with anti-ARP15 monoclonal antibody 1R. (B) LNCaP cells stained with anti-β-integrin monoclonal antibody.

FIG. 7 shows immunohistochemical staining with anti-ARP15 monoclonal antibody 1R. (A) Prostate cancer tissue section stained with anti-ARP15. (B) Normal prostate tissue section stained with anti-ARP15.

### DETAILED DESCRIPTION OF THE INVENTION

This invention is directed to the discovery of androgen regulated prostate (ARP) expressed nucleic acid molecules. The androgen regulated prostate expressed nucleic acid molecules and encoded gene products are useful as diagnostic markers for neoplastic conditions and other disorders of the prostate, and, further, are targets for therapy as described further herein below.

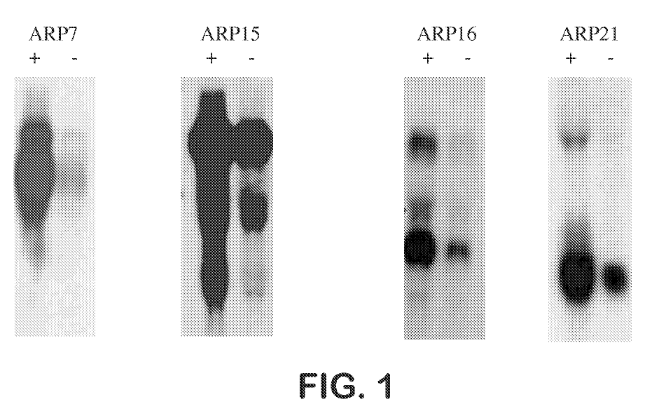

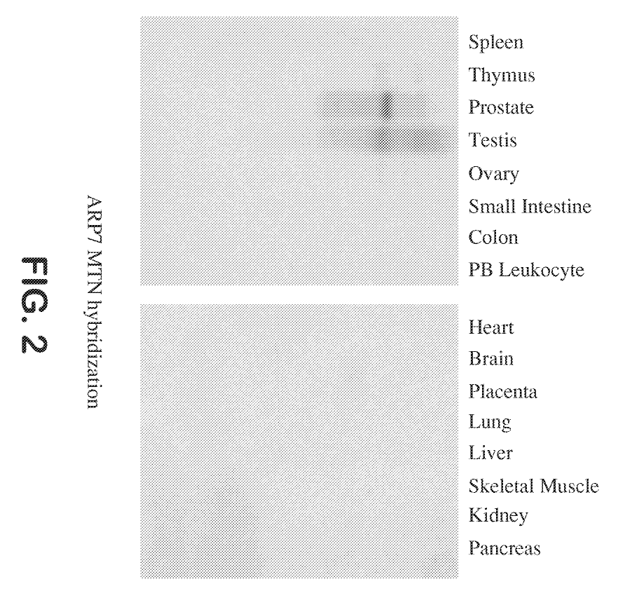

As disclosed herein in Example I, the ARP7 cDNA is an androgen-regulated sequence. The ARP7 nucleic acid molecule, which contains 5470 nucleotides, is provided herein as SEQ ID NO: 1. Nucleotides 474 to 4967 encode a polypeptide of 1498 amino acids (SEQ ID NO: 2). As shown in FIG. 1, ARP7 mRNA is dramatically up-regulated by androgen in starved LNCaP cells. As further shown in FIG. 2, ARP7 is most highly expressed in the prostate with little or no detectable expression in other tissues.

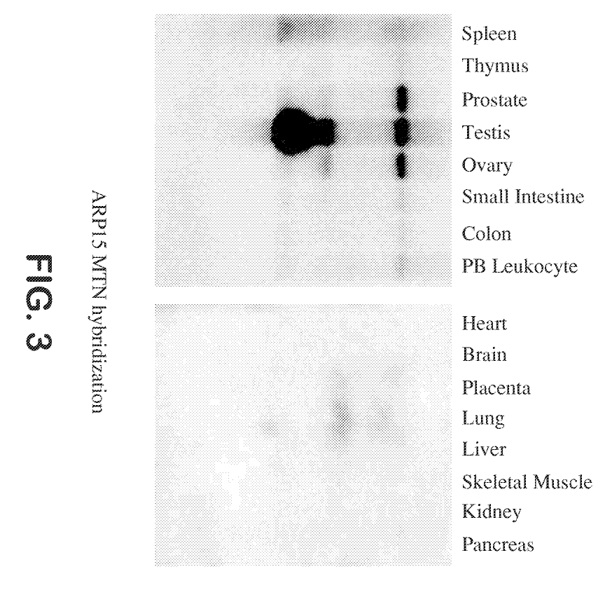

As further disclosed herein, the ARP15 cDNA also is a human androgen-regulated sequence (see FIG. 1). The human ARP15 nucleic acid molecule (SEQ ID NO: 3), which contains 3070 nucleotides, has an open reading frame from nucleotide 253 to 1527. The ARP15 cDNA sequence is predicted to encode a polypeptide of 425 amino acids (SEQ ID NO: 4) with at least three transmembrane domains. As shown in FIG. 3, ARP15 is expressed in prostate tissue and also expressed in testis and ovary.

As further disclosed herein, the ARP16 cDNA is up-regulated by androgen in human prostate cells. The human ARP16 cDNA, shown herein as SEQ ID NO: 5, has 2161 nucleotides with an open reading frame from nucleotide 138 to 1601. Furthermore, the human ARP16 is a polypeptide of 488 amino acids (SEQ ID NO: 4) with at least eight predicted transmembrane domains. As shown in FIG. 1, ARP16 mRNA is dramatically up-regulated by androgen in starved LNCaP cells.

Additional androgen regulated cDNAs also are disclosed herein. ARP8 is a human sequence up-regulated by androgen in prostate cells. The human ARP8 cDNA (SEQ ID NO: 7) contains 2096 nucleotides with an open reading frame from nucleotide 1 to 1728; the encoded human ARP8 polypeptide (SEQ ID NO: 8) has 576 amino acids. The nucleic acid sequence of another human androgen-regulated cDNA expressed in prostate, ARP9, is disclosed herein as SEQ ID NO: 9. The ARP9 nucleic acid sequence disclosed herein has 2568 nucleotides with an open reading frame from nucleotide 559 to 2232. The encoded human ARP9 polypeptide (SEQ ID NO: 10) has 558 residues and is predicted to include at least four transmembrane domains. The ARP13 cDNA also increased in response to androgen in the LNCaP cell line. The ARP13 nucleotide sequence (SEQ ID NO: 11) has 2920 nucleotides with an open reading frame from nucleotide 141 to 1022. The human ARP13 polypeptide has the 294 amino acid sequence shown herein as SEQ ID NO: 12 and is predicted to include at least one transmembrane domain. The ARP20 nucleotide sequence shown herein as SEQ ID NO: 13 also was identified as positively regulated in response to androgen in LNCaP cells. The human ARP20 nucleotide sequence has 1095 nucleotides with an open reading frame from nucleotide 113 to 661; the human ARP20 polypeptide is shown herein as SEQ ID NO: 14.

As further disclosed herein, ARP24, ARP26, ARP28, ARP30, ARP33 and ARP11 also are androgen regulated cDNAs expressed in the LNCaP prostate cell line. The ARP24 cDNA sequence shown herein as SEQ ID NO: 15 contains 3007 nucleotides with an open reading frame from nucleotide 38 to 1378; the encoded human ARP24 polypeptide (SEQ ID NO: 16) has 447 amino acids predicted to encode at least four transmembrane domains. The ARP26 cDNA sequence shown herein as SEQ ID NO: 17 is a sequence of 3937 nucleotides with an open reading frame from nucleotide 240 to 1013. The corresponding androgen-regulated human ARP26 polypeptide (SEQ ID NO: 18) has 258 residues. Furthermore, the ARP28 cDNA sequence, shown herein as SEQ ID NO: 19, is a sequence of 1401 nucleotides with an open reading frame from nucleotide 45 to 1085 and is predicted to encode the 347 amino acid human ARP28 polypeptide (SEQ ID NO: 20) with at least three transmembrane domains. The androgen-regulated cDNA ARP30 has a sequence (SEQ ID NO: 21) of 3318 nucleotides; the human ARP30 polypeptide (SEQ ID NO: 22), a protein of 601 amino acids, is encoded by an open reading frame positioned at nucleotides 252 to 2054 of SEQ ID NO: 21. As further disclosed herein, the androgen-regulated ARP33 cDNA has a nucleic acid sequence (SEQ ID NO: 23) of 1690 nucleotides with an open reading frame from nucleotide 98 to 1313. The human ARP33 polypeptide, a protein of 405 residues shown herein as SEQ ID NO: 24, is predicted to include at least one transmembrane domain. In addition, the human ARP11 cDNA has a nucleic acid sequence (SEQ ID NO: 33) of 3067 nucleotides with an open reading frame from nucleotides 790 to 1805 that encodes the human ARP11 polypeptide disclosed herein as SEQ ID NO: 34.

As further disclosed herein, ARP6, ARP10, ARP12, ARP18, ARP19, ARP21, ARP22 and ARP29 also are androgen-regulated sequences expressed in prostate cells. The human ARP6 cDNA sequence is shown herein as a 504 nucleotide sequence (SEQ ID NO: 25); the human ARP10 cDNA sequence is shown herein as a 2189 nucleotide sequence (SEQ ID NO: 26); the human ARP12 cDNA sequence is shown herein as a 2576 nucleotide sequence (SEQ ID NO: 27); and the human ARP18 cDNA sequence is shown herein as a 521 nucleotide sequence (SEQ ID NO: 28). Furthermore, the human ARP19 cDNA sequence is shown herein as a 644 nucleotide sequence (SEQ ID NO: 29); the human ARP21 cDNA sequence is shown herein as a 1460 nucleotide sequence (SEQ ID NO: 30); the human ARP22 cDNA sequence is shown herein as a 774 nucleotide sequence (SEQ ID NO: 31); and the human ARP29 cDNA sequence is shown herein as a 386 nucleotide sequence (SEQ ID NO: 32).

Based on these novel prostate-expressed sequences, the invention provides methods for diagnosing prostate neoplastic conditions. An ARP nucleic acid molecule or polypeptide of the invention can be used alone or in combination with other molecules as a specific marker for prostate cells or prostate neoplastic conditions.

The present invention provides a substantially pure ARP7 nucleic acid molecule which includes the nucleotide sequence shown as SEQ ID NO: 1. The invention also provides a substantially pure ARP7 nucleic acid molecule that has at least 10 contiguous nucleotides of nucleotides 1-445 of SEQ ID NO: 1.

The present invention also provides a substantially pure ARP15 nucleic acid molecule that includes the nucleotide sequence shown as SEQ ID NO: 3. In addition, the invention provides a substantially pure ARP15 nucleic acid molecule which has at least 10 contiguous nucleotides of nucleotides 1-86 of SEQ ID NO: 3.

The present invention additionally provides a substantially pure ARP16 nucleic acid molecule that contains a nucleic acid sequence encoding an ARP16 polypeptide having at least 90% amino acid identity with SEQ ID NO: 6. Such a nucleic acid molecule can encode, for example, the amino acid sequence shown as SEQ ID NO:6. In one embodiment, an ARP16 nucleic acid molecule of the invention includes the nucleotide sequence shown as SEQ ID NO:5. Further provided by the invention is a substantially pure ARP16 nucleic acid molecule that includes at least 10 contiguous nucleotides of nucleotides 1-1531 of SEQ ID NO: 5.

Also provided herein is a substantially pure ARP8 nucleic acid molecule that contains a nucleic acid sequence encoding an ARP8 polypeptide having at least 65% amino acid identity with SEQ ID NO: 8. Such a substantially pure ARP8 nucleic acid molecule can encode, for example, the amino acid sequence shown as SEQ ID NO: 8. In one embodiment, an ARP8 nucleic acid molecule of the invention has the nucleotide sequence shown as SEQ ID NO: 7. Also provided herein is a substantially pure ARP8 nucleic acid molecule which includes at least 10 contiguous nucleotides of nucleotides 1-349 of SEQ ID NO: 7.

The present invention further provides a substantially pure ARP9 nucleic acid molecule that includes a nucleic acid sequence encoding an ARP9 polypeptide having at least 65% amino acid identity with SEQ ID NO: 10. A substantially pure ARP9 nucleic acid molecule of the invention can encode, for example, the amino acid sequence shown as SEQ ID NO: 10. In one embodiment, an ARP9 nucleic acid molecule includes the nucleotide sequence shown as SEQ ID NO:9. The invention also provides a substantially pure ARP9 nucleic acid molecule that includes at least 10 contiguous nucleotides of nucleotides 697-745 of SEQ ID NO: 9.

The present invention also provides a substantially pure ARP13 nucleic acid molecule that includes a nucleic acid sequence encoding an ARP13 polypeptide having at least 90% amino acid identity with SEQ ID NO: 12. Such a substantially pure ARP13 nucleic acid molecule can encode, for example, the amino acid sequence shown as SEQ ID NO: 12. In one embodiment, a substantially pure ARP13 nucleic acid molecule of the invention has the nucleotide sequence shown as SEQ ID NO: 11.

The present invention further provides a substantially pure ARP26 nucleic acid which includes the nucleotide sequence shown as SEQ ID NO: 17. The invention also provides a substantially pure ARP26 nucleic acid molecule of the invention that includes at least 10 contiguous nucleotides of nucleotides 1404-1516 of SEQ ID NO: 17.

Further provided herein is a substantially pure ARP30 nucleic acid molecule that includes a nucleic acid sequence encoding an ARP30 polypeptide having at least 30% amino acid identity with SEQ ID NO: 22. A substantially pure ARP30 nucleic acid molecule of the invention can encode, for example, the amino acid sequence shown as SEQ ID NO: 22, and, in one embodiment, includes the nucleotide sequence shown as SEQ ID NO: 21. Also provided herein is a substantially pure ARP30 nucleic acid molecule that includes at least 10 contiguous nucleotides of nucleotides 1-132, nucleotides 832-1696, or nucleotides 2346-2796 of SEQ ID NO: 21.

The present invention also provides a substantially pure ARP11 nucleic acid molecule that contains the nucleotide sequence shown as SEQ ID NO: 33.

In addition, there is provided a substantially pure ARP11 nucleic acid molecule which contains at least 10 contiguous nucleotides of nucleotides 1-458 of SEQ ID NO: 33.

The invention also provides a substantially pure ARP6 nucleic acid molecule that includes the nucleotide sequence shown as SEQ ID NO: 25. Further provided herein is a substantially pure ARP6 nucleic acid molecule that contains at least 10 contiguous nucleotides of nucleotides 505-526 of SEQ ID NO: 25.

The present invention further provides a substantially pure ARP12 nucleic acid molecule that contains the nucleotide sequence shown as SEQ ID NO: 27. In addition, the invention provides a substantially pure ARP12 nucleic acid molecule that contains at least 10 contiguous nucleotides of nucleotides 1635-1659 of SEQ ID NO: 27.

The invention also provides a substantially pure ARP19 nucleic acid molecule that includes the nucleotide sequence shown as SEQ ID NO: 29. Furthermore, there is provided herein a substantially pure ARP19 nucleic acid molecule which has at least 10 contiguous nucleotides of nucleotides 1-31 or 478-644 of SEQ ID NO: 29.

In addition, the present invention provides a substantially pure ARP22 nucleic acid molecule which includes the nucleotide sequence shown as SEQ ID NO: 31. In addition, the invention provides a substantially pure ARP22 nucleic acid molecule that has at least 10 contiguous nucleotides of nucleotides 1-73 or 447-464 of SEQ ID NO: 31.

The nucleic acid molecules of the invention corresponding to unique sequences are useful in a variety of diagnostic procedures which employ probe hybridization methods. One advantage of employing nucleic acid hybridization in diagnostic procedures is that very small amounts of sample can be used because the analyte nucleic acid molecule can be amplified to many copies by, for example, polymerase chain reaction (PCR) or other well known methods for nucleic acid molecule amplification and synthesis.

As used herein, the term “nucleic acid molecule” means a single- or double-stranded DNA or RNA molecule including, for example, genomic DNA, cDNA and mRNA. The term is intended to include nucleic acid molecules of both synthetic and natural origin. A nucleic acid molecule of natural origin can be derived from any animal, such as a human, non-human primate, mouse, rat, rabbit, bovine, porcine, ovine, canine, feline, or amphibian, or from a lower eukaryote. A nucleic acid molecule of the invention can be of linear, circular or branched configuration, and can represent either the sense or antisense strand, or both, of a native nucleic acid molecule. A nucleic acid molecule of the invention can further incorporate a detectable moiety such as a radiolabel, a fluorochrome, a ferromagnetic substance, a luminescent tag or a detectable moiety such as biotin.

As used herein, the term “substantially pure nucleic acid molecule” means a nucleic acid molecule that is substantially free from cellular components or other contaminants that are not the desired molecule. A substantially pure nucleic acid molecule can also be sufficiently homogeneous so as to resolve as a band by gel electrophoresis, and generate a nucleotide sequence profile consistent with a predominant species.

In particular embodiments, the present invention provides a substantially pure ARP7 nucleic acid molecule which has at least 10 contiguous nucleotides of nucleotides 1-445 of SEQ ID NO: 1; a substantially pure ARP15 nucleic acid molecule which has at least 10 contiguous nucleotides of nucleotides 1-86 of SEQ ID NO: 3; a substantially pure ARP16 nucleic acid molecule which has at least 10 contiguous nucleotides of nucleotides 1-1531 of SEQ ID NO: 5; a substantially pure ARP8 nucleic acid molecule which has at least 10 contiguous nucleotides of nucleotides 1-349 of SEQ ID NO: 7; a substantially pure ARP9 nucleic acid molecule which has at least 10 contiguous nucleotides of nucleotides 697-745 of SEQ ID NO: 9; a substantially pure ARP26 nucleic acid molecule which has at least 10 contiguous nucleotides of nucleotides 1404-1516 of SEQ ID NO: 17; a substantially pure ARP30 nucleic acid molecule which has at least 10 contiguous nucleotides of nucleotides 1-132, at least 10 contiguous nucleotides of nucleotides 832-1696, or at least 10 contiguous nucleotides of nucleotides 2346-2796 of SEQ ID NO: 21; and a substantially pure ARP11 nucleic acid molecule which has at least 10 contiguous nucleotides of nucleotides 1-458 of SEQ ID NO: 33.

The invention also provides a substantially pure ARP6 nucleic acid molecule which has at least 10 contiguous nucleotides of nucleotides 505-526 of SEQ ID NO: 25; a substantially pure ARP12 nucleic acid molecule which has at least 10 contiguous nucleotides of nucleotides 1635-1659 of SEQ ID NO: 27; a substantially pure ARP19 nucleic acid molecule which has at least 10 contiguous nucleotides of nucleotides 1-31 or at least 10 contiguous nucleotides of nucleotides 478-644 of SEQ ID NO: 29; and a substantially pure ARP22 nucleic acid molecule which has at least 10 contiguous nucleotides of nucleotides 1-73 or at least 10 contiguous nucleotides of nucleotides 447-464 of SEQ ID NO: 31.

Such a nucleic acid molecule having “at least 10 contiguous nucleotides” is a portion of a full-length nucleic acid molecule having the ability to selectively hybridize with the parent nucleic acid molecule. As used herein, the term “selectively hybridize” means an ability to bind the parent nucleic acid molecule without substantial cross-reactivity with a molecule that is not the parent nucleic acid molecule. Therefore, the term selectively hybridize includes specific hybridization where there is little or no detectable cross-reactivity with other nucleic acid molecules. The term also includes minor cross-reactivity with other molecules provided hybridization to the parent nucleic acid molecule is distinguishable from hybridization to the cross-reactive species. Thus, a nucleic acid molecule of the invention can be used, for example, as a PCR primer to selectively amplify a parent nucleic acid molecule; as a selective primer for 5′ or 3′ RACE to determine additional 5′ or 3′ sequence of a parent nucleic acid molecule; as a selective probe to identify or isolate a parent nucleic acid molecule on a RNA or DNA blot, or within a genomic or cDNA library; or as a selective inhibitor of transcription or translation of an ARP in a tissue, cell or cell extract.

A nucleic acid molecule of the invention includes at least 10 contiguous nucleotides corresponding to the reference nucleic acid molecule, and can include at least 11, 12, 13, 14, 15, 16, 17, 18, 19, 20, 25, 30, 35, 40, 45 or 50 nucleotides and, if desired, can include at least 100, 200, 300, 400, 500 or 1000 nucleotides or up to the full length of the reference nucleic acid molecule. Nucleic acid molecules of such lengths are able to selectively hybridize with the subject nucleic acid molecule in a variety of detection formats described herein.

As used herein, the term “substantially the nucleotide sequence” in reference to a nucleic acid molecule or nucleic acid probe of the invention includes sequences having one or more additions, deletions or substitutions with respect to the reference sequence, so long as the nucleic acid molecule retains its ability to selectively hybridize with the subject nucleic acid molecule.

Nucleic acid molecules of the invention are useful, in part, as hybridization probes in diagnostic procedures. The nucleic acid molecules can be as long as the full length transcript or as short as about 10 to 15 nucleotides, for example, 15 to 18 nucleotides in length. A nucleic acid molecule of the invention that is not a full-length sequence can correspond to a coding region or an untranslated region. The particular application and degree of desired specificity will be one consideration well known to those skilled in the art in selecting a nucleic acid molecule for a particular application. For example, if it is desired to detect an ARP and other related species, the probe can correspond to a coding sequence and be used in low stringency hybridization conditions. Alternatively, using high stringency conditions with a probe of the invention will select a specific ARP7, ARP15, ARP16, ARP8, ARP9, ARP13, ARP26, ARP30, ARP11, ARP6, ARP12, ARP19 or ARP22 nucleic acid molecule. Untranslated region sequences corresponding to an ARP transcript also can be used to construct probes since there is little evolutionary pressure to conserve non-coding domains. Nucleic acid molecules as small as 15 nucleotides are statistically unique sequences within the human genome. Therefore, fragments of 15 nucleotides or more of the ARP sequences disclosed herein as SEQ ID NOS: 1, 3, 5, 7, 9, 11, 17, 21, 25, 27, 29, 31 and 33 can be constructed from essentially any region of an ARP cDNA, mRNA or promoter/regulatory region and be capable of uniquely hybridizing to ARP DNA or RNA.

A nucleic acid molecule of the invention can be produced recombinantly or chemically synthesized using methods well known in the art. Additionally, an ARP nucleic acid molecule can be labeled with a variety of detectable labels including, for example, radioisotopes, fluorescent tags, reporter enzymes, biotin and other ligands for use as a probe in a hybridization method. Such detectable labels can additionally be coupled with, for example, calorimetric or photometric indicator substrate for spectrophotometric detection. Methods for labeling and detecting nucleic acid molecules are well known in the art and can be found described in, for example, Sambrook et al., Molecular Cloning: A Laboratory Manual, 2nd ed., Cold Spring Harbor Press, Plainview, N.Y. (1989), and Ausubel et al., Current Protocols in Molecular Biology (Supplement 47), John Wiley & Sons, New York (1999).

The nucleic acid molecules of the invention can be hybridized under various stringency conditions readily determined by one skilled in the art. Depending on the particular assay, one skilled in the art can readily vary the stringency conditions to optimize detection of an ARP nucleic acid molecule.

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, at least 75% identity, at least 85% identity; or at least 90% identity with the parent or target nucleic acid sequence. Moderately stringent conditions are conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C.

The term low stringency hybridization means conditions equivalent to hybridization in 10% formamide, 5× Denhart's solution, 6×SSPE, 0.2% SDS at 22° C., followed by washing in 1×SSPE, 0.2% SDS, at 37° C. Denhart's solution contains 1% Ficoll, 1% polyvinylpyrrolidine, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., supra, 1999). Nucleic acid molecules encoding polypeptides hybridize under moderately stringent or high stringency conditions to substantially the entire sequence, or substantial portions, for example, typically at least 15-30 nucleotides of an ARP nucleic acid sequence.

The invention also provides a modification of an ARP nucleotide sequence that hybridizes under moderately stringent conditions to an ARP nucleic acid molecule, for example, an ARP nucleic acid molecule referenced herein as SEQ ID NO: 1, 3, 5, 7, 9, 11, 17, 21, 25, 27, 29, 31 or 33. Modifications of ARP nucleotide sequences, where the modification has at least 60% identity to an ARP nucleotide sequence, are also provided. The invention also provides modification of an ARP nucleotide sequence having at least 65% identity, at least 70% identity, at least 75% identity, at least 80% identity, at least 85% identity, at least 90% identity, or at least 95% identity to SEQ ID NO: 1, 3, 5, 7, 9, 11, 17, 21, 25, 27, 29, 31 or 33.

Identity of any two nucleic acid sequences can be determined by those skilled in the art based, for example, on a BLAST 2.0 computer alignment, using default parameters. BLAST 2.0 searching is available at http://www.ncbi.nlm.nih.gov/gorf/bl2.html., as described by Tatiana et al., FEMS Microbiol Lett. 174:247-250 (1999); Altschul et al., Nucleic Acids Res., 25:3389-3402 (1997).

The present invention further provides substantially pure ARP polypeptides encoded by the prostate-expressed nucleic acid molecules of the invention. In particular, the present invention provides a substantially pure ARP16 polypeptide that contains an amino acid sequence having at least 90% amino acid identity with SEQ ID NO: 6. An ARP16 polypeptide of the invention can include, for example, the amino acid sequence shown as SEQ ID NO: 6. Also provided by the invention is a substantially pure ARP16 polypeptide fragment which has at least eight contiguous amino acids of SEQ ID NO: 6. In one embodiment, an ARP16 polypeptide fragment of the invention has at least eight contiguous amino acids of residues 1-465 of SEQ ID NO: 6.

The present invention further provides a substantially pure ARP8 polypeptide that contains an amino acid sequence having at least 65% amino acid identity with SEQ ID NO: 8. Such an ARP8 polypeptide can have, for example, the amino acid sequence shown as SEQ ID NO: 8. In addition, there is provided herein a substantially pure ARP8 polypeptide fragment, which includes at least eight contiguous amino acids of residues 1-116 of SEQ ID NO: 8. In one embodiment, the ARP8 fragment has at least eight contiguous amino acids of residues 249-576 of SEQ ID NO: 8.

The invention also provides a substantially pure ARP9 polypeptide that includes an amino acid sequence having at least 65% amino acid identity with SEQ ID NO: 10. Such an ARP9 polypeptide can have, for example, the amino acid sequence shown as SEQ ID NO: 10. Substantially pure ARP9 polypeptide fragments also are provided herein. The ARP9 fragments of the invention have at least eight contiguous amino acids of residues 1-83 of SEQ ID NO: 10. In one embodiment, such an ARP9 fragment has at least eight contiguous amino acids of residues 47-62 of SEQ ID NO: 10.

Also provided herein is a substantially pure ARP13 polypeptide, which has an amino acid sequence having at least 90% amino acid identity with SEQ ID NO: 12. As an example, a substantially pure ARP13 polypeptide of the invention can have the amino acid sequence shown as SEQ ID NO: 12. The invention additionally provides a substantially pure ARP13 polypeptide fragment that includes at least eight contiguous amino acids of SEQ ID NO: 12.

The invention also provides a substantially pure ARP20 polypeptide that includes an amino acid sequence having at least 55% amino acid identity with SEQ ID NO: 14. Such an ARP20 polypeptide can have, for example, the amino acid sequence shown as SEQ ID NO: 14. Also provided herein is a substantially pure ARP20 polypeptide fragment including at least eight contiguous amino acids of SEQ ID NO: 14.

Further provided herein is a substantially pure ARP24 polypeptide that includes an amino acid sequence having at least 30% amino acid identity with SEQ ID NO: 16. A substantially pure ARP24 polypeptide of the invention can have, for example, the amino acid sequence shown as SEQ ID NO: 16. The invention also provides a substantially pure ARP24 polypeptide fragment which contains at least eight contiguous amino acids of SEQ ID NO: 16.

Also provided herein is a substantially pure ARP30 polypeptide that contains an amino acid sequence having at least 30% amino acid identity with SEQ ID NO: 22. In one embodiment, a substantially pure ARP30 polypeptide of the invention includes the amino acid sequence shown as SEQ ID NO: 22. The invention also provides a substantially pure ARP30 polypeptide fragment that has at least eight contiguous amino acids of SEQ ID NO: 22.

The invention also provides a substantially pure ARP33 polypeptide that includes an amino acid sequence having at least 70% amino acid identity with SEQ ID NO: 24. Such a substantially pure ARP33 polypeptide can have, for example, the amino acid sequence shown as SEQ ID NO: 24. Also provided herein is a substantially pure ARP33 polypeptide fragment that includes at least eight contiguous amino acids of residues 1-132 or 251-405 of SEQ ID NO: 24.

The invention further provides a substantially pure ARP11 polypeptide which contains an amino acid sequence having at least 75% amino acid identity with SEQ ID NO: 34. Such an ARP11 polypeptide can include, for example, the amino acid sequence shown as SEQ ID NO: 34. Also provided is a substantially pure ARP11 polypeptide fragment containing at least eight contiguous amino acids of SEQ ID NO: 34.

Exemplary polypeptide fragments include those fragments having amino acids 1 to 8, 2 to 9, 3 to 10, etc., of SEQ ID NO: 6, 8, 10, 12, 14, 16, 22, 24 or 34. The invention also encompasses other polypeptide fragments which are potential antigenic fragments capable of eliciting an immune response, and thereby generating antibodies selective for an ARP16, ARP8, ARP9, ARP13, ARP20, ARP24, ARP30, ARP33 or ARP11 polypeptide or polypeptide fragment of the invention. It is understood that polypeptide fragments of other lengths also can be useful, for example, a polypeptide having at least nine, ten, 11, 12, 13, 14, 15, 16, 17, 18, 19, 20, 25, 30, 35, 40, 45 or more contiguous amino acids of the amino acid sequence disclosed herein as SEQ ID NO: 6, residues 1-465 of SEQ ID NO: 6; residues 1-116 of SEQ ID NO: 8; residues 249-576 of SEQ ID NO: 8; residues 1-83 of SEQ ID NO: 10; residues 47-62 of SEQ ID NO: 10; the amino acid sequence disclosed herein as SEQ ID NO: 12; the amino acid sequence disclosed herein as SEQ ID NO: 14; the amino acid sequence disclosed herein as SEQ ID NO: 16; the amino acid sequence disclosed herein as SEQ ID NO: 22; residues 1-132 of the amino acid sequence disclosed herein as SEQ ID NO: 24; residues 251-405 of the amino acid sequence disclosed herein as SEQ ID NO: 24; or the amino acid sequence disclosed herein as SEQ ID NO: 34. It is understood that polypeptide fragments encompassed by the invention further include, for example, polypeptide fragments having at least 50, 100, 150, 200, 250, 300, 350, 400, 450, 500, 550, 600, 650, 700, 750, 800, 850, 900, 950, 1000, 1050, 1110, 1150, 1200, 1250, 1300, 1350, 1400, 1450 or 1500 amino acids beginning at residue 1, 50, 100, 150, 200, 250, 300, 350, 400, 450, 500, 550, 600, 650, 700, 750, 800, 850, 900, 950, 1000, 1050, 1110, 1150, 1200, 1250, 1300, 1350, 1400, 1450 of SEQ ID NO: 6, SEQ ID NO: 8, SEQ ID NO: 10, SEQ ID NO: 12, SEQ ID NO: 14, SEQ ID NO: 16, SEQ ID NO: 22, SEQ ID NO: 24 or SEQ ID NO: 34. Such polypeptide fragments can be useful to produce binding agents or in any of the compositions or diagnostic or therapeutic methods of the invention.

The term “ARP16 polypeptide” as used herein, means a polypeptide that is structurally similar to a human ARP16 (SEQ ID NO: 6) and that has at least one biological activity of human ARP16. Such an ARP16 polypeptide has 90% or more amino acid sequence identity to SEQ ID NO:16 and can have, for example, 92%, 94%, 96%, 98%, 99% or more sequence identity to human ARP16 (SEQ ID NO: 6). Percent amino acid identity can be determined using Clustal W version 1.7 (Thompson et al., Nucleic Acids Res. 22:4673-4680 (1994)).

Thus, it is clear to the skilled person that the term “ARP16 polypeptide” encompasses polypeptides with one or more naturally occurring or non-naturally occurring amino acid substitutions, deletions or insertions as compared to SEQ ID NO: 6, provided that the peptide has at least 90% amino acid identity with SEQ ID NO: 6 and retains at least one biological activity of human ARP16. An ARP16 polypeptide can be, for example, a naturally occurring variant of human ARP16 (SEQ ID NO: 6); a species homolog such as a porcine, bovine or primate homolog; an ARP16 polypeptide mutated by recombinant techniques, and the like. In view of the above definition, it is clear to the skilled person that the mouse protein shown in Genbank accession BAB28556, which shares 87% amino acid identity with human ARP16 (SEQ ID NO: 6), is not encompassed by the invention.

The term “ARP8 polypeptide” as used herein, means a polypeptide that is structurally similar to a human ARP8 (SEQ ID NO: 8) and that has at least one biological activity of human ARP8. Such an ARP8 polypeptide has 65% or more amino acid sequence identity to SEQ ID NO:5 and can have, for example 70%, 75%, 80%, 85%, 90%, 95% or more amino acid sequence identity to human ARP8 (SEQ ID NO: 8). Percent amino acid identity can be determined using Clustal W version 1.7 as described above.

Thus, the term “ARP8 polypeptide” encompasses polypeptides with one or more naturally occurring or non-naturally occurring amino acid substitutions, deletions or insertions as compared to SEQ ID NO:8, provided that the peptide has at least 65% amino acid identity with SEQ ID NO: 8 and retains at least one biological activity of human ARP8. An ARP8 polypeptide can be, for example, a naturally occurring variant of human ARP8 (SEQ ID NO: 8); a species homolog such as a non-mammalian or mammalian homolog, for example, a murine, bovine or primate homolog; an ARP8 polypeptide mutated by recombinant techniques; and the like. The polypeptide encoded by murine protein (Genbank accession BAB28455), which shares 62% amino acid identity with human ARP8 (SEQ ID NO: 8), is not encompassed by the invention.

The term “ARP9 polypeptide” as used herein, means a polypeptide that is structurally similar to a human ARP9 (SEQ ID NO: 10) and that has at least one biological activity of human ARP9. Such an ARP9 polypeptide has 65% or more amino acid sequence identity to SEQ ID NO: 10 and can have, for example, 70%, 75%, 80%, 85%, 90%, 95% or more amino acid sequence identity to human ARP9 (SEQ ID NO: 10). Percent amino acid identity can be determined using Clustal W version 1.7 as described above.

Thus, the term “ARP9 polypeptide” encompasses polypeptides with one or more naturally occurring or non-naturally occurring amino acid substitutions, deletions or insertions as compared to SEQ ID NO: 10, provided that the peptide has at least 65% amino acid identity with SEQ ID NO: 10 and retains at least one biological activity of human ARP9. An ARP9 polypeptide can be, for example, a naturally occurring variant of human ARP9 (SEQ ID NO: 10); a species homolog such as a non-mammalian or mammalian homolog, for example, a murine, bovine or primate homolog; an ARP9 polypeptide mutated by recombinant techniques; and the like. The polypeptide encoded by Genbank accession NP_{—}071769), which shares 63% amino acid identity with human ARP9 (SEQ ID NO: 10), is not encompassed by the invention.

The term “ARP13 polypeptide” as used herein, means a polypeptide that is structurally similar to a human ARP13 (SEQ ID NO: 12) and that has at least one biological activity of human ARP13. Such an ARP13 polypeptide has 90% or more amino acid sequence identity to SEQ ID NO: 12 and can have, for example, 92%, 94%, 96%, 98%, 99% or more sequence identity to human ARP13 (SEQ ID NO: 12). Percent amino acid identity can be determined using Clustal W version 1.7 (Thompson et al., supra, 1994).

The term “ARP13 polypeptide” encompasses polypeptides with one or more naturally occurring or non-naturally occurring amino acid substitutions, deletions or insertions as compared to SEQ ID NO: 12, provided that the peptide has at least 90% amino acid identity with SEQ ID NO: 12 and retains at least one biological activity of human ARP13. An ARP13 polypeptide can be, for example, a naturally occurring variant of human ARP13 (SEQ ID NO: 12); a species homolog such as a non-mammalian or mammalian homolog, for example, a murine, bovine or primate homolog; an ARP13 polypeptide mutated by recombinant techniques, and the like. In view of the above definition, it is clear to the skilled person that the polypeptide encoded by Genbank accession BAB29190, which shares 86% amino acid identity with human ARP13 (SEQ ID NO: 12), is not encompassed by the invention.

The term “ARP20 polypeptide” as used herein, means a polypeptide that is structurally similar to a human ARP20 (SEQ ID NO: 14) and that has at least one biological activity of human ARP20. Such an ARP20 polypeptide has 55% or more amino acid sequence identity to SEQ ID NO:12 and can have, for example, 60%, 65%, 70%, 75%, 80%, 85%, 90%, 95% or more sequence identity to human ARP20 (SEQ ID NO: 14). Percent amino acid identity can be determined using Clustal W version 1.7 (Thompson et al., supra, 1994).

The term “ARP20 polypeptide” encompasses polypeptides with one or more naturally occurring or non-naturally occurring amino acid substitutions, deletions or insertions as compared to SEQ ID NO: 14, provided that the peptide has at least 55% amino acid identity with SEQ ID NO: 14 and retains at least one biological activity of human ARP20. An ARP20 polypeptide can be, for example, a naturally occurring variant of human ARP20 (SEQ ID NO: 14); a species homolog such as a non-mammalian or mammalian homolog, for example, a murine, bovine or primate homolog; an ARP20 polypeptide mutated by recombinant techniques, and the like. In view of the above definition, it is clear to the skilled person that the polypeptide encoded by Genbank accession AAL27184, which shares 50% amino acid identity with human ARP20 (SEQ ID NO: 14), is not encompassed by the invention.

The term “ARP24 polypeptide” as used herein, means a polypeptide that is structurally similar to a human ARP24 (SEQ ID NO: 16) and that has at least one biological activity of human ARP24. Such an ARP24 polypeptide has 30% or more amino acid sequence identity to SEQ ID NO:14 and can have, for example, 45%, 50%, 55%, 60%, 65%, 70%, 75%, 80%, 85%, 90%, 95% or more sequence identity to human ARP24 (SEQ ID NO: 16). Percent amino acid identity can be determined using Clustal W version 1.7 (Thompson et al., supra, 1994).

The term “ARP24 polypeptide” encompasses polypeptides with one or more naturally occurring or non-naturally occurring amino acid substitutions, deletions or insertions as compared to SEQ ID NO: 16, provided that the peptide has at least 30% amino acid identity with SEQ ID NO: 16 and retains at least one biological activity of human ARP24. An ARP24 polypeptide can be, for example, a naturally occurring variant of human ARP24 (SEQ ID NO: 16); a species homolog such as a non-mammalian or mammalian homolog, for example, a murine, bovine or primate homolog; an ARP24 polypeptide mutated by recombinant techniques, and the like.

Similarly, the term “ARP30 polypeptide” as used herein, means a polypeptide that is structurally similar to a human ARP30 (SEQ ID NO: 22) and that has at least one biological activity of human ARP30. Such an ARP30 polypeptide has 30% or more amino acid sequence identity to SEQ ID NO:20 and can have, for example, 45%, 50%, 55%, 60%, 65%, 70%, 75%, 80%, 85%, 90%, 95% or more sequence identity to human ARP30 (SEQ ID NO: 22). Percent amino acid identity can be determined using Clustal W version 1.7 (Thompson et al., supra, 1994).

The term “ARP30 polypeptide” encompasses polypeptides with one or more naturally occurring or non-naturally occurring amino acid substitutions, deletions or insertions as compared to SEQ ID NO: 22, provided that the peptide has at least 30% amino acid identity with SEQ ID NO: 22 and retains at least one biological activity of human ARP30. An ARP30 polypeptide can be, for example, a naturally occurring variant of human ARP30 (SEQ ID NO: 22); a species homolog such as a non-mammalian or mammalian homolog, for example, a murine, bovine or primate homolog; an ARP30 polypeptide mutated by recombinant techniques, and the like.

The term “ARP33 polypeptide” as used herein, means a polypeptide that is structurally similar to a human ARP33 (SEQ ID NO: 24) and that has at least one biological activity of human ARP33. Such an ARP33 polypeptide has 70% or more amino acid sequence identity to SEQ ID NO:22 and can have, for example, 75%, 80%, 85%, 90%, 95% or more sequence identity to human ARP33 (SEQ ID NO: 24). Percent amino acid identity can be determined using Clustal W version 1.7 (Thompson et al., supra, 1994).

The term “ARP33 polypeptide” encompasses polypeptides with one or more naturally occurring or non-naturally occurring amino acid substitutions, deletions or insertions as compared to SEQ ID NO: 24, provided that the peptide has at least 70% amino acid identity with SEQ ID NO: 24 and retains at least one biological activity of human ARP33. An ARP33 polypeptide can be, for example, a naturally occurring variant of human ARP33 (SEQ ID NO: 24); a species homolog including mammalian and non-mammalian homologs and murine, bovine, and primate homologs; an ARP33 polypeptide mutated by recombinant techniques, and the like. In view of the above, it is understood that the murine polypeptide encoded by Genbank accession NP_{—}033387, which shares 67% amino acid identity with human ARP33 (SEQ ID NO: 24), is not encompassed by the invention.

The term “ARP11 polypeptide” as used herein, means a polypeptide that is structurally similar to a human ARP11 (SEQ ID NO: 34) and that has at least one biological activity of human ARP11. Such an ARP11 polypeptide has 75% or more amino acid sequence identity to SEQ ID NO: 34 and can have, for example, 80%, 85%, 90%, 95% or more sequence identity to human ARP11 (SEQ ID NO: 34). Percent amino acid identity can be determined using Clustal W version 1.7 (Thompson et al., Nucleic Acids Res. 22:4673-4680 (1994)).

Thus, it is clear to the skilled person that the term “ARP11 polypeptide” encompasses polypeptides with one or more naturally occurring or non-naturally occurring amino acid substitutions, deletions or insertions as compared to SEQ ID NO: 34, provided that the peptide has at least 75% amino acid identity with SEQ ID NO: 34 and retains at least one biological activity of human ARP11. An ARP11 polypeptide can be, for example, a naturally occurring variant of human ARP11 (SEQ ID NO: 34); a species homolog such as a porcine, bovine or primate homolog; an ARP11 polypeptide mutated by recombinant techniques, and the like. In view of the above definition, it is clear to the skilled person that the mouse protein shown in Genbank accession BAB28028, which shares 72% amino acid identity with human ARP11 (SEQ ID NO: 34), is not encompassed by the invention.

Modifications to the ARP16, ARP8, ARP9, ARP13, ARP20, ARP24, ARP30, ARP33 and ARP11 polypeptides of SEQ ID NOS: 6, 8, 10, 12, 14, 16, 22, 24 and 34 that are encompassed within the invention include, for example, an addition, deletion, or substitution of one or more conservative or non-conservative amino acid residues; substitution of a compound that mimics amino acid structure or function; or addition of chemical moieties such as amino or acetyl groups.

The present invention also provides a variety of binding agents that selectively bind an ARP polypeptide of the invention. Such binding agents encompass, but are not limited to, polyclonal and monoclonal antibodies and binding portions thereof.

The present invention provides an ARP16 binding agent which includes a molecule that selectively binds at least eight contiguous amino acids of SEQ ID NO: 6. In one embodiment, such an ARP16 binding agent selectively binds at least eight contiguous amino acids of residues 1-465 of SEQ ID NO: 6. In another embodiment, the binding agent is an antibody.

Also provided herein is an ARP8 binding agent which includes a molecule that selectively binds at least eight contiguous amino acids of residues 1-116 of SEQ ID NO: 8, for example, an antibody that selectively binds at least eight contiguous amino acids of residues 1-116 of SEQ ID NO: 8. In addition, the invention provides a binding agent which includes a molecule that selectively binds at least eight contiguous amino acids of residues 249-576 of SEQ ID NO: 8. Such an ARP8 binding agent can be, for example, an antibody.

The invention also provides an ARP9 binding agent that includes a molecule that selectively binds at least eight contiguous amino acids of residues 1-83 of SEQ ID NO: 10. In one embodiment, the ARP9 binding agent includes a molecule that selectively binds at least eight contiguous amino acids of residues 47-62 of SEQ ID NO: 10. An ARP9 binding agent of the invention can be, for example, an antibody.

Further provided herein is an ARP13 binding agent which includes a molecule that selectively binds at least eight contiguous amino acids of SEQ ID NO: 12. ARP13 binding agents include, without limitation, antibodies.

The invention also provides an ARP20 binding agent which contains a molecule that selectively binds at least eight contiguous amino acids of SEQ ID NO: 14. In one embodiment, the ARP20 binding agent is an antibody.

In addition, there is provided herein an ARP24 binding agent that includes a molecule that selectively binds at least eight contiguous amino acids of SEQ ID NO: 16. In one embodiment, the ARP24 binding agent is an antibody.

In addition, there is provided herein an ARP30 binding agent, which includes a molecule that selectively binds at least eight contiguous amino acids of SEQ ID NO: 22. ARP30 binding agents encompass but are not limited to antibodies.

The present invention also provides an ARP33 binding agent that includes a molecule that selectively binds at least eight contiguous amino acids of residues 1-132 or at least eight contiguous amino acids of 251-405 of SEQ ID NO: 24. In a particular embodiment, the ARP33 binding agent is an antibody.

Further provided herein is an ARP11 binding agent, which includes a molecule that selectively binds at least eight contiguous amino acids of SEQ ID NO: 34. ARP11 binding agents encompass, but are not limited to, antibodies.

As used herein, the term “binding agent” when used in reference to a specified ARP polypeptide, means a compound, including a simple or complex organic molecule, a metal containing compound, carbohydrate, peptide, protein, peptidomimetic, glycoprotein, lipoprotein, lipid, nucleic acid molecule, antibody, or the like that selectively binds an ARP16, ARP8, ARP9, ARP13, ARP20, ARP24, ARP30, ARP33 or ARP11 polypeptide, or the specified fragment thereof. For example, a binding agent can be a polypeptide that selectively binds with high affinity or avidity to the specified ARP polypeptide, without substantial cross-reactivity to other unrelated polypeptides. The affinity of a binding agent that selectively binds an ARP polypeptide generally is greater than about 10⁵ M⁻¹ and can be greater than about 10⁶ M⁻¹. A binding agent also can bind with high affinity; such an agent generally binds with an affinity greater than 10⁸ M⁻¹ to 10⁹ M⁻¹. Specific examples of such selective binding agents include a polyclonal or monoclonal antibody selective for an ARP16, ARP8, ARP9, ARP13, ARP20, ARP24, ARP30, ARP33 or ARP11 polypeptide, or the specified fragment thereof; or a nucleic acid molecule, nucleic acid analog, or small organic molecule, identified, for example, by affinity screening of the appropriate library. For certain applications, a binding agent can be utilized that preferentially recognizes a particular conformational or post-translationally modified state of the specified ARP polypeptide. The binding agent can be labeled with a detectable moiety, if desired, or rendered detectable by specific binding to a detectable secondary binding agent.

As used herein, the term “antibody” is used in its broadest sense to mean polyclonal and monoclonal antibodies, including antigen binding fragments of such antibodies. As used herein, the term antigen means a native or synthesized fragment of a polypeptide of the invention. Such an antibody of the invention, or antigen binding fragment of such an antibody, is characterized by having specific binding activity for an ARP16, ARP8, ARP9, ARP13, ARP20, ARP24, ARP30, ARP33, or ARP11 polypeptide, or the specified fragment thereof, of at least about 1×10⁵ M⁻¹. Thus, Fab, F(ab′)₂, Fd and Fv fragments of an anti-ARP antibody, which retain specific binding activity for an ARP polypeptide of the invention, or fragment thereof, are included within the definition of an antibody. Specific binding activity can be readily determined by one skilled in the art, for example, by comparing the binding activity of the antibody to the specified ARP polypeptide, or fragment thereof, versus a control polypeptide that does not include a polypeptide of the invention. Methods of preparing polyclonal or monoclonal antibodies are well known to those skilled in the art (see, for example, Harlow and Lane, Antibodies: A Laboratory Manual, Cold Spring Harbor Laboratory Press (1988)).

The term “antibody” also includes naturally occurring antibodies as well as non-naturally occurring antibodies, including, for example, single chain antibodies, chimeric, bi-functional and humanized antibodies, as well as antigen-binding fragments thereof. Such non-naturally occurring antibodies can be constructed using solid phase peptide synthesis, produced recombinantly or obtained, for example, by screening combinatorial libraries consisting of variable heavy chains and variable light chains as described by Huse et al. (Science 246:1275-1281 (1989)). These and other methods of making, for example, chimeric, humanized, CDR-grafted, single chain, and bi-functional antibodies are well known to those skilled in the art (Winter and Harris, Immunol. Today 14:243-246 (1993); Ward et al., Nature 341:544-546 (1989); Harlow and Lane, supra, 1988); Hilyard et al., Protein Engineering: A practical approach (IRL Press 1992); Borrabeck, Antibody Engineering, 2d ed. (Oxford University Press 1995)).

An antibody of the invention can be prepared using as an immunogen an ARP16, ARP8, ARP9, ARP13, ARP20, ARP24, ARP30, ARP33 or ARP11 polypeptide, which can be prepared from natural sources or produced recombinantly, or a polypeptide fragment containing at least 8 contiguous amino acids of SEQ ID NO: 6, at least 8 contiguous amino acids of residues 1-116 or 249-576 of SEQ ID NO: 8; at least 8 contiguous amino acids of residues 1-83 or 47-62 of SEQ ID NO: 10; at least 8 contiguous amino acids of SEQ ID NO: 12, 14, 16 or 22; at least 8 contiguous amino acids of residues 1-132 of SEQ ID NO: 24; at least 8 contiguous amino acids of residues 251-405 of SEQ ID NO: 24; or at least 8 contiguous amino acids of SEQ ID NO: 34.

Such polypeptide fragments are functional antigenic fragments if the antigenic peptides can be used to generate an antibody selective for an ARP polypeptide of the invention. As is well known in the art, a non-immunogenic or weakly immunogenic ARP polypeptide of the invention, or polypeptide fragment thereof, can be made immunogenic by coupling the hapten to a carrier molecule such as bovine serum albumin (BSA) or keyhole limpet hemocyanin (KLH). Various other carrier molecules and methods for coupling a hapten to a carrier molecule are well known in the art (see, for example, Harlow and Lane, supra, 1988). An immunogenic ARP polypeptide fragment of the invention can also be generated by expressing the peptide portion as a fusion protein, for example, to glutathione S transferase (GST), polyHis or the like. Methods for expressing peptide fusions are well known to those skilled in the art (Ausubel et al., Current Protocols in Molecular Biology (Supplement 47), John Wiley & Sons, New York (1999)).

The present invention also provides a method of diagnosing or predicting susceptibility to a prostate neoplastic condition in an individual. The method is practiced by contacting a sample from the individual with an ARP7 nucleic acid molecule containing at least 10 contiguous nucleotides of SEQ ID NO: 1; determining a test expression level of ARP7 RNA in the sample; and comparing the test expression level to a non-neoplastic control expression level of ARP7 RNA, where an altered test expression level as compared to the control expression level indicates the presence of a prostate neoplastic condition in the individual. In one embodiment, the method is practiced with a prostate tissue sample. In another embodiment, the method is practiced with a sample of blood, urine or semen. In a further embodiment, the method is practiced with an ARP7 nucleic acid molecule that has a length of 15 to 35 nucleotides. In yet a further embodiment, the invention is practiced with an ARP7 nucleic acid molecule that has at least 10 contiguous nucleotides of nucleotides 1-445 of SEQ ID NO: 1.

Also provided herein is a method of diagnosing or predicting susceptibility to a prostate neoplastic condition in an individual by contacting a sample from the individual with an ARP15 nucleic acid molecule that includes at least 10 contiguous nucleotides of SEQ ID NO: 3; determining a test expression level of ARP15 RNA in the sample; and comparing the test expression level to a non-neoplastic control expression level of ARP15 RNA, where an altered test expression level as compared to the control expression level indicates the presence of a prostate neoplastic condition in the individual. A sample useful in such a method of the invention can include, for example, prostate tissue, or can be, for example, blood, urine or semen. An ARP15 nucleic acid molecule useful in a method of the invention can have a length of, for example, 15 to 35 nucleotides. In one embodiment, the ARP15 nucleic acid molecule has at least 10 contiguous nucleotides of nucleotides 1-86 of SEQ ID NO: 3.

The invention also provides a method of diagnosing or predicting susceptibility to a prostate neoplastic condition in an individual by contacting a sample from the individual with an ARP16 nucleic acid molecule containing at least 10 contiguous nucleotides of SEQ ID NO: 5; determining a test expression level of ARP16 RNA in the sample; and comparing the test expression level to a non-neoplastic control expression level of ARP16 RNA, where an altered test expression level as compared to the control expression level indicates the presence of a prostate neoplastic condition in the individual. Samples useful in the methods of the invention include, for example, prostate tissue samples as well as samples of blood, urine or semen. In one embodiment, a method of the invention is practiced with an ARP16 nucleic acid molecule which has a length of 15 to 35 nucleotides. In another embodiment, a method of the invention is practiced with an ARP16 nucleic acid molecule that has at least 10 contiguous nucleotides of nucleotides 1-1531 of SEQ ID NO: 5.

The invention additionally provides a method of diagnosing or predicting susceptibility to a prostate neoplastic condition in an individual by contacting a sample from the individual with an ARP8 nucleic acid molecule containing at least 10 contiguous nucleotides of SEQ ID NO:7; determining a test expression level of ARP8 RNA in the sample; and comparing the test expression level to a non-neoplastic control expression level of ARP8 RNA, where an altered test expression level as compared to the control expression level indicates the presence of a prostate neoplastic condition in the individual. In one embodiment, the sample includes prostate tissue. In other embodiments, the sample is blood, urine or semen. In a further embodiment, the ARP8 nucleic acid molecule has a length of 15 to 35 nucleotides. In yet a further embodiment, the ARP8 nucleic acid molecule includes at least 10 contiguous nucleotides of nucleotides 1-349 of SEQ ID NO: 7.

Further provided herein is a method of diagnosing or predicting susceptibility to a prostate neoplastic condition in an individual by contacting a sample from the individual with an ARP9 nucleic acid molecule that includes at least 10 contiguous nucleotides of SEQ ID NO: 9; determining a test expression level of ARP9 RNA in the sample; and comparing the test expression level to a non-neoplastic control expression level of ARP9 RNA, where an altered test expression level as compared to the control expression level indicates the presence of a prostate neoplastic condition in the individual. In one embodiment, a method of the invention is practiced with a sample that includes prostate tissue. In other embodiments, a method of the invention is practiced with a sample of blood, urine or semen. In a further embodiment, a method of the invention is practiced with an ARP9 nucleic acid molecule having a length of 15 to 35 nucleotides. In yet a further embodiment, a method of the invention is practiced with an ARP9 nucleic acid molecule which has at least 10 contiguous nucleotides of nucleotides 697-745 of SEQ ID NO: 9.

The invention also provides a method of diagnosing or predicting susceptibility to a prostate neoplastic condition in an individual by contacting a sample from the individual with an ARP13 nucleic acid molecule that includes at least 10 contiguous nucleotides of SEQ ID NO: 11; determining a test expression level of ARP13 RNA in the sample; and comparing the test expression level to a non-neoplastic control expression level of ARP13 RNA, where an altered test expression level as compared to the control expression level indicates the presence of a prostate neoplastic condition in the individual. A method of the invention can be practiced, for example, with a sample which includes prostate tissue or, for example, with a blood, urine or semen sample. A variety of ARP13 nucleic acid molecules are useful in the methods of the invention including ARP13 nucleic acid molecules of 15 to 35 nucleotides in length. There further is provided herein a method of diagnosing or predicting susceptibility to a prostate neoplastic condition in an individual by contacting a sample from the individual with an ARP20 nucleic acid molecule which includes at least 10 contiguous nucleotides of SEQ ID NO: 13; determining a test expression level of ARP20 RNA in the sample; and comparing the test expression level to a non-neoplastic control expression level of ARP20 RNA, where an altered test expression level as compared to the control expression level indicates the presence of a prostate neoplastic condition in the individual. Samples useful in a method of the invention include prostate tissue, blood, urine and semen. In one embodiment, a method of the invention is practiced with an ARP20 nucleic acid molecule having a length of 15 to 35 nucleotides. Also provided herein is a method of diagnosing or predicting susceptibility to a prostate neoplastic condition in an individual. The method includes the steps of contacting a sample from the individual with an ARP24 nucleic acid molecule containing at least 10 contiguous nucleotides of SEQ ID NO: 15; determining a test expression level of ARP24 RNA in the sample; and comparing the test expression level to a non-neoplastic control expression level of ARP24 RNA, where an altered test expression level as compared to the control expression level indicates the presence of a prostate neoplastic condition in the individual. In one embodiment, a method of the invention is practiced with a sample containing prostate tissue. In other embodiments, a method of the invention is practiced with a sample of blood, urine or semen. In yet another embodiment, the method is practiced with an ARP24 nucleic acid molecule that is 15 to 35 nucleotides in length.

Also provided herein is a method of diagnosing or predicting susceptibility to a prostate neoplastic condition in an individual. A method of the invention includes the steps of contacting a sample from the individual with an ARP26 nucleic acid molecule containing at least 10 contiguous nucleotides of SEQ ID NO: 17; determining a test expression level of ARP26 RNA in the sample; and comparing the test expression level to a non-neoplastic control expression level of ARP26 RNA, where an altered test expression level as compared to the control expression level indicates the presence of a prostate neoplastic condition in the individual. Samples useful in a method of the invention include prostate tissue, blood, urine and semen. In one embodiment, a method of the invention is practiced with an ARP26 nucleic acid molecule having a length of 15 to 35 nucleotides. In another embodiment, a method of the invention is practiced with an ARP26 nucleic acid molecule having at least 10 contiguous nucleotides of nucleotides 1404-1516 of SEQ ID NO: 17.

The invention further provides a method of diagnosing or predicting susceptibility to a prostate neoplastic condition in an individual, in which a sample from the individual is contacted with an ARP28 nucleic acid molecule containing at least 10 contiguous nucleotides of SEQ ID NO: 19; a test expression level of ARP28 RNA in the sample is determined; and the test expression level is compared to a non-neoplastic control expression level of ARP28 RNA, where an altered test expression level as compared to the control expression level indicates the presence of a prostate neoplastic condition in the individual. In one embodiment, the sample contacted with an ARP28 nucleic acid molecule contains prostate tissue. In other embodiments, the sample is blood, urine or semen sample. In a further embodiment, the ARP28 nucleic acid molecule has a length of 15 to 35 nucleotides.

The invention also provides herein a method of diagnosing or predicting susceptibility to a prostate neoplastic condition in an individual by contacting a sample from the individual with an ARP30 nucleic acid molecule containing at least 10 contiguous nucleotides of nucleotides 1-1829 or nucleotides 2346-3318 of SEQ ID NO: 21; determining a test expression level of ARP30 RNA in the sample; and comparing the test expression level to a non-neoplastic control expression level of ARP30 RNA, where an altered test expression level as compared to the control expression level indicates the presence of a prostate neoplastic condition in the individual. In one embodiment, a method of the invention is practiced with a sample containing prostate tissue. In other embodiments, a method of the invention is practiced with a blood, urine or semen sample. In a further embodiment, a method of the invention is practiced with an ARP30 nucleic acid molecule having a length of 15 to 35 nucleotides. In yet a further embodiment, a method of the invention is practiced with an ARP30 nucleic acid molecule that includes at least 10 contiguous nucleotides of nucleotides 1-132, nucleotides 832-1696, or nucleotides 2346-2796 of SEQ ID NO: 21.

The invention also provides a method of diagnosing or predicting susceptibility to a prostate neoplastic condition in an individual by contacting a sample from the individual with an ARP33 nucleic acid molecule that includes at least 10 contiguous nucleotides of SEQ ID NO: 23; determining a test expression level of ARP33 RNA in the sample; and comparing the test expression level to a non-neoplastic control expression level of ARP33 RNA, where an altered test expression level as compared to the control expression level indicates the presence of a prostate neoplastic condition in the individual. Samples useful in the invention can include, for example, prostate tissue. Samples useful in the invention also can be samples of blood, urine or semen. A variety of ARP33 nucleic acid molecules are useful in the methods of the invention including, for example, ARP33 nucleic acid molecules of 15 to 35 nucleotides in length.

Also provided herein is a method of diagnosing or predicting susceptibility to a prostate neoplastic condition in an individual by contacting a sample from the individual with an ARP11 nucleic acid molecule containing at least 10 contiguous nucleotides of nucleotides 1-458 of SEQ ID NO: 33; determining a test expression level of ARP11 RNA in the sample; and comparing the test expression level to a non-neoplastic control expression level of ARP11 RNA, where an altered test expression level as compared to the control expression level indicates the presence of a prostate neoplastic condition in the individual. A sample useful for diagnosing or predicting susceptibility to a prostate neoplastic condition according to a method of the invention can be, for example, a sample of prostate tissue or a sample of blood, urine or semen. In one embodiment, a method of the invention is practiced with an ARP11 nucleic acid molecule having a length of 15 to 35 nucleotides.

The invention additionally provides a method of diagnosing or predicting susceptibility to a prostate neoplastic condition in an individual by contacting a sample from the individual with an ARP6 nucleic acid molecule containing at least 10 contiguous nucleotides of SEQ ID NO: 25; determining a test expression level of ARP6 RNA in the sample; and comparing the test expression level to a non-neoplastic control expression level of ARP6 RNA, where an altered test expression level as compared to the control expression level indicates the presence of a prostate neoplastic condition in the individual. In one embodiment, the method is practiced with a prostate tissue sample. In another embodiment, the method is practiced with a sample of blood, urine or semen. In a further embodiment, the method is practiced with an ARP6 nucleic acid molecule having a length of 15 to 35 nucleotides. In yet a further embodiment, the method is practiced with an ARP6 nucleic acid molecule which contains at least 10 contiguous nucleotides of nucleotides 505-526 of SEQ ID NO: 25.

The invention further provides a method of diagnosing or predicting susceptibility to a prostate neoplastic condition in an individual by contacting a sample from the individual with an ARP11 nucleic acid molecule containing at least 10 contiguous nucleotides of SEQ ID NO: 26; determining a test expression level of ARP10 RNA in the sample; and comparing the test expression level to a non-neoplastic control expression level of ARP10 RNA, where an altered test expression level as compared to the control expression level indicates the presence of a prostate neoplastic condition in the individual. In one embodiment, the method is practiced with a sample containing prostate tissue. In other embodiments, the method is practiced with a blood, urine or semen sample. In a further embodiment, the method is practiced with an ARP10 nucleic acid molecule of 15 to 35 nucleotides in length.

Also provided herein is a method of diagnosing or predicting susceptibility to a prostate neoplastic condition in an individual by contacting a sample from the individual with an ARP12 nucleic acid molecule containing at least 10 contiguous nucleotides of nucleotides 1-1659 or 2176-2576 of SEQ ID NO: 27; determining a test expression level of ARP12 RNA in the sample; and comparing the test expression level to a non-neoplastic control expression level of ARP12 RNA, where an altered test expression level as compared to the control expression level indicates the presence of a prostate neoplastic condition in the individual. In one embodiment, the method is practiced with a sample containing prostate tissue. In other embodiments, the method is practiced with a blood, urine or semen sample. In a further embodiment, a method of the invention is practiced with an ARP12 nucleic acid molecule that has a length of 15 to 35 nucleotides. In yet a further embodiment, a method of the invention is practiced with an ARP12 nucleic acid molecule that contains at least 10 contiguous nucleotides of nucleotides 1635-1659 of SEQ ID NO: 27.

The present invention additionally provides a method of diagnosing or predicting susceptibility to a prostate neoplastic condition in an individual by contacting a sample from the individual with an ARP18 nucleic acid molecule containing at least 10 contiguous nucleotides of SEQ ID NO: 28; determining a test expression level of ARP18 RNA in the sample; and comparing the test expression level to a non-neoplastic control expression level of ARP18 RNA, where an altered test expression level as compared to the control expression level indicates the presence of a prostate neoplastic condition in the individual. A method of the invention can be practiced, for example, with a sample containing prostate tissue, or, for example, with a sample of blood, urine or semen. A variety of ARP18 nucleic acid molecules are useful in the methods of the invention. In one embodiment, the invention is practiced with an ARP18 nucleic acid molecule which has a length of 15 to 35 nucleotides.

The invention further provides a method of diagnosing or predicting susceptibility to a prostate neoplastic condition in an individual by contacting a sample from the individual with an ARP19 nucleic acid molecule containing at least 10 contiguous nucleotides of SEQ ID NO: 29; determining a test expression level of ARP19 RNA in the sample; and comparing the test expression level to a non-neoplastic control expression level of ARP19 RNA, where an altered test expression level as compared to the control expression level indicates the presence of a prostate neoplastic condition in the individual. A method of the invention can be practiced, for example, with a sample containing prostate tissue, or, for example, with a sample of blood, urine or semen. A variety of ARP19 nucleic acid molecules are useful in the methods of the invention, for example, ARP19 nucleic acid molecules of 15 to 35 nucleotides in length. In a particular embodiment, a method of the invention is practiced with an ARP19 nucleic acid molecule which has at least 10 contiguous nucleotides of nucleotides 1-31 or 478-644 of SEQ ID NO: 29.

The present invention also provides a method of diagnosing or predicting susceptibility to a prostate neoplastic condition in an individual by contacting a sample from the individual with an ARP21 nucleic acid molecule containing at least 10 contiguous nucleotides of SEQ ID NO: 30; determining a test expression level of ARP21 RNA in the sample; and comparing the test expression level to a non-neoplastic control expression level of ARP21 RNA, where an altered test expression level as compared to the control expression level indicates the presence of a prostate neoplastic condition in the individual. Samples useful in the invention include, without limitation, those containing prostate tissue as well as blood, urine and semen samples. In one embodiment, a method of the invention is practiced with an ARP21 nucleic acid molecule having a length of 15 to 35 nucleotides.

Further provided by the present invention is a method of diagnosing or predicting susceptibility to a prostate neoplastic condition in an individual by contacting a sample from the individual with an ARP22 nucleic acid molecule containing at least 10 contiguous nucleotides of SEQ ID NO: 31; determining a test expression level of ARP22 RNA in the sample; and comparing the test expression level to a non-neoplastic control expression level of ARP22 RNA, where an altered test expression level as compared to the control expression level indicates the presence of a prostate neoplastic condition in the individual. In one embodiment, the method is practiced with a sample containing prostate tissue. In other embodiments, the method is practiced with a blood, urine or semen sample. In a further embodiment, a method of the invention is practiced with an ARP22 nucleic acid molecule having a length of 15 to 35 nucleotides. In yet a further embodiment, a method of the invention is practiced with an ARP22 nucleic acid molecule that has at least 10 contiguous nucleotides of nucleotides 1-73 or 447-464 of SEQ ID NO: 31.

The present invention also provides a method of diagnosing or predicting susceptibility to a prostate neoplastic condition in an individual by contacting a sample from the individual with an ARP29 nucleic acid molecule containing at least 10 contiguous nucleotides of SEQ ID NO: 32; determining a test expression level of ARP29 RNA in the sample; and comparing the test expression level to a non-neoplastic control expression level of ARP29 RNA, where an altered test expression level as compared to the control expression level indicates the presence of a prostate neoplastic condition in the individual. In one embodiment, the method is practiced with a sample containing prostate tissue. In other embodiments, the method is practiced with a sample of blood, urine or semen. In a further embodiment, a method of the invention is practiced with an ARP29 nucleic acid molecule which has a length of 15 to 35 nucleotides.

In the diagnostic methods of the invention, the sample can be, for example, a prostate tissue, or can be, for example, a fluid such as blood, urine or semen. The non-neoplastic control expression level can be determined, for example, using a normal prostate cell or an androgen-dependent cell line.

As described herein, the term “prostate neoplastic condition” means a benign or malignant or metastatic prostate lesion of proliferating cells. For example, primary prostate tumors are classified into stages TX, T0, T1, T2, T3, and T4. Metastatic prostate cancer is classified into stages D1, D2, and D3. The term further includes prostate neoplasm. Each of the above conditions is encompassed within the term “prostate neoplastic condition.”

As used herein, the term “sample” means any biological fluid, cell, tissue, organ or portion thereof, that includes or potentially includes an ARP nucleic acid molecule. The term sample includes materials present in an individual as well as materials obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid molecule. A sample can be prepared by methods known in the art suitable for the particular format of the detection method.

As used herein, the term “test expression level” is used in reference to ARP RNA expression or to ARP polypeptide expression as discussed below and means the extent, amount or rate of synthesis of the specified ARP RNA or polypeptide. The amount or rate of synthesis can be determined by measuring the accumulation or synthesis of the specified ARP RNA or polypeptide, or by measuring an activity associated with a polypeptide of the invention.

As used herein, an “altered test expression level” means a test expression level that is either elevated or reduced as compared to a control expression level. One skilled in the art understands that such an elevation or reduction is not within the inherent variability of the assay and generally is an expression level that is at least two-fold elevated or reduced. An altered test expression level can be, for example, two-fold, five-fold, ten-fold, 100-fold, 200-fold, or 1000-fold increased in the extent, amount or rate of synthesis of the specified RNA or polypeptide as compared to a control expression level of the specified ARP RNA or polypeptide. An altered test expression level also can be, for example, two-fold, five-fold, ten-fold, 100-fold, 200-fold, or 1000-fold decreased in the extent, amount or rate of synthesis of the specified ARP RNA or polypeptide compared to a control expression level of the same ARP RNA or polypeptide.

As used herein, the term “non-neoplastic control expression level” means an ARP RNA expression level or to an ARP polypeptide expression level as discussed below used as a baseline for comparison to a test expression level. For example, a suitable control expression level can be the expression level of ARP nucleic acid or polypeptide from a non-neoplastic prostate cell or a fluid sample obtained from a normal individual. Another suitable non-neoplastic control is a prostate cell line that is androgen-dependent. It is understood that ARP nucleic acid or polypeptide expression levels determined in cell lines generally are determined under androgen-depleted growth conditions which can correlate to non-neoplastic control expression levels. The response of an androgen-depleted androgen-dependent prostate cell line to androgen stimulation will be indicative of ARP nucleic acid or polypeptide expression levels in neoplastic cells. The control expression level can be determined simultaneously with one or more test samples or, alternatively, expression levels can be established for a particular type of sample and standardized to internal or external parameters such as protein or nucleic acid content, cell number or mass of tissue. Such standardized control samples can then be directly compared with results obtained from the test sample. As indicated above, an increase of two-fold or more, for example, of a test expression level of the specified ARP nucleic acid or polypeptide indicates the presence of a prostate neoplastic condition or pathology in the tested individual.

A detectable label can be useful in a method of the invention and refers to a molecule that renders a nucleic acid molecule of the invention detectable by an analytical method. An appropriate detectable label depends on the particular assay format; such labels are well known by those skilled in the art. For example, a detectable label selective for a nucleic acid molecule can be a complementary nucleic acid molecule, such as a hybridization probe, that selectively hybridizes to the nucleic acid molecule. A hybridization probe can be labeled with a measurable moiety, such as a radioisotope, fluorochrome, chemiluminescent marker, biotin, or other moiety known in the art that is measurable by analytical methods. A detectable label also can be a nucleic acid molecule without a measurable moiety. For example, PCR or RT-PCR primers can be used without conjugation to selectively amplify all or a desired portion of the nucleic acid molecule. The amplified nucleic acid molecules can then be detected by methods known in the art.

The present invention also provide diagnostic methods that rely on a binding agent that selectively binds the specified ARP polypeptide. In particular, the present invention provides a method of diagnosing or predicting susceptibility to a prostate neoplastic condition in an individual by contacting a specimen from the individual with an ARP7 binding agent that selectively binds an ARP7 polypeptide; determining a test expression level of ARP7 polypeptide in the specimen; and comparing the test expression level to a non-neoplastic control expression level of ARP7 polypeptide, where an altered test expression level as compared to the control expression level indicates the presence of a prostate neoplastic condition in the individual. A method of the invention can be practiced with a specimen that includes, for example, prostate tissue, or with a specimen which is blood, serum, urine or semen. If desired, a method of the invention for diagnosing or predicting susceptibility to a prostate neoplastic condition can be practiced with an ARP7 binding agent which is an antibody. In one embodiment, a method of the invention is practiced with an ARP7 binding agent that selectively binds human ARP7 (SEQ ID NO: 2).

The invention also provides a method of diagnosing or predicting susceptibility to a prostate neoplastic condition in an individual by contacting a specimen from the individual with an ARP15 binding agent that selectively binds an ARP15 polypeptide; determining a test expression level of ARP15 polypeptide in the specimen; and comparing the test expression level to a non-neoplastic control expression level of ARP15 polypeptide, where an altered test expression level as compared to the control expression level indicates the presence of a prostate neoplastic condition in the individual. A specimen useful in such a method can include, for example, prostate tissue, or can be, for example, blood, serum, urine or semen. In one embodiment, the ARP15 binding agent that selectively binds the ARP15 polypeptide is an antibody. In another embodiment, a method of the invention is practiced with an ARP15 binding agent that selectively binds human ARP15 (SEQ ID NO: 4).

Also provided herein is a method of diagnosing or predicting susceptibility to a prostate neoplastic condition in an individual by contacting a specimen from the individual with an ARP16 binding agent that selectively binds an ARP16 polypeptide; determining a test expression level of ARP16 polypeptide in the specimen; and comparing the test expression level to a non-neoplastic control expression level of ARP16 polypeptide, where an altered test expression level as compared to the control expression level indicates the presence of a prostate neoplastic condition in the individual. A specimen useful for diagnosing or predicting susceptibility to a prostate neoplastic condition can include, for example, prostate tissue, or can be, for example, a specimen of blood, serum, urine or semen. In one embodiment, the ARP16 binding agent is an antibody. In a further embodiment, a method of the invention is practiced with an ARP16 binding agent that selectively binds human ARP16 (SEQ ID NO: 6). In another embodiment, a method of the invention is practiced with an ARP16 binding agent that selectively binds at least eight contiguous amino acids of residues 1-465 of SEQ ID NO: 6.

There is further provided herein a method of diagnosing or predicting susceptibility to a prostate neoplastic condition in an individual by contacting a specimen from the individual with an ARP8 binding agent that selectively binds an ARP8 polypeptide; determining a test expression level of ARP8 polypeptide in the specimen; and comparing the test expression level to a non-neoplastic control expression level of ARP8 polypeptide, where an altered test expression level as compared to the control expression level indicates the presence of a prostate neoplastic condition in the individual. A method of the invention can be practiced, for example, with a specimen that includes prostate tissue, or with a specimen which is blood, serum, urine or semen.

In one embodiment, the ARP8 binding agent is an antibody. In another embodiment, the ARP8 binding agent selectively binds at least eight contiguous amino acids of human ARP8 (SEQ ID NO: 8). In a further embodiment, the ARP8 binding agent selectively binds at least eight contiguous amino acids of residues 1-116 of SEQ ID NO: 8. In yet a further embodiment, the ARP8 binding agent selectively binds residues 249-576 of SEQ ID NO: 8.

The present invention also provides a method of diagnosing or predicting susceptibility to a prostate neoplastic condition in an individual, in which a specimen from the individual is contacted with an ARP9 binding agent that selectively binds an ARP9 polypeptide; a test expression level of ARP9 polypeptide in the specimen is determined; and the test expression level is compared to a non-neoplastic control expression level of ARP9 polypeptide, where an altered test expression level as compared to the control expression level indicates the presence of a prostate neoplastic condition in the individual. A method of the invention can be practiced with a specimen containing, for example, prostate tissue, or, for example, with a blood, serum, urine or semen specimen. If desired, a method of the invention can be practiced with an ARP9 binding agent which is an antibody. In one embodiment, a method of the invention is practiced with an ARP9 binding agent that selectively binds at least eight contiguous amino acids of human ARP9 (SEQ ID NO: 10).

The invention also provides a method of diagnosing or predicting susceptibility to a prostate neoplastic condition in an individual by contacting a specimen from the individual with an ARP13 binding agent that selectively binds an ARP13 polypeptide; determining a test expression level of ARP13 polypeptide in the specimen; and comparing the test expression level to a non-neoplastic control expression level of ARP13 polypeptide, where an altered test expression level as compared to the control expression level indicates the presence of a prostate neoplastic condition in the individual. A variety of specimens are useful in a method of the invention for diagnosing or predicting susceptibility to a prostate neoplastic condition, including, but not limited to, prostate tissue, blood, serum, urine and semen. An ARP13 binding agent useful in a method of the invention can be, for example, an antibody. An ARP13 binding agent useful in the invention also can be an ARP13 binding agent that selectively binds at least eight contiguous amino acids of human ARP13 (SEQ ID NO: 12).

Further provided herein is a method of diagnosing or predicting susceptibility to a prostate neoplastic condition in an individual by contacting a specimen from the individual with an ARP20 binding agent that selectively binds an ARP20 polypeptide; determining a test expression level of ARP20 polypeptide in the specimen; and comparing the test expression level to a non-neoplastic control expression level of ARP20 polypeptide, where an altered test expression level as compared to the control expression level indicates the presence of a prostate neoplastic condition in the individual. In one embodiment, a method of the invention is practiced with a specimen of prostate tissue. In another embodiment, a method of the invention is practiced with a blood, serum, urine or semen specimen. In a further embodiment, a method of the invention is practiced with an ARP20 binding agent which is an antibody. In yet a further embodiment, a method of the invention is practiced with an ARP20 binding agent that selectively binds at least eight contiguous amino acids of human ARP20 (SEQ ID NO: 14).

The invention also provides a method of diagnosing or predicting susceptibility to a prostate neoplastic condition in an individual by contacting a specimen from the individual with an ARP24 binding agent that selectively binds an ARP24 polypeptide; determining a test expression level of ARP24 polypeptide in the specimen; and comparing the test expression level to a non-neoplastic control expression level of ARP24 polypeptide, where an altered test expression level as compared to the control expression level indicates the presence of a prostate neoplastic condition in the individual. Samples useful in a method of the invention include prostate tissue, blood, urine and semen. In one embodiment, a method of the invention is practiced with an ARP24 nucleic acid molecule having a length of 15 to 35 nucleotides. In another embodiment, a method of the invention is practiced with an ARP24 binding agent that selectively binds at least eight contiguous amino acids of human ARP24 (SEQ ID NO: 16).

The invention also provides a method of diagnosing or predicting susceptibility to a prostate neoplastic condition in an individual by contacting a specimen from the individual with an ARP26 binding agent that selectively binds an ARP26 polypeptide; determining a test expression level of ARP26 polypeptide in the specimen; and comparing the test expression level to a non-neoplastic control expression level of ARP26 polypeptide, where an altered test expression level as compared to the control expression level indicates the presence of a prostate neoplastic condition in the individual. A specimen useful in the invention can include, for example, prostate tissue, or can be, for example, a blood, serum, urine or semen specimen. In one embodiment, the ARP26 binding agent is an antibody. In another embodiment, the ARP26 binding agent selectively binds at least eight contiguous amino acids of human ARP26 (SEQ ID NO: 18).

The invention further provides herein a method of diagnosing or predicting susceptibility to a prostate neoplastic condition in an individual by contacting a specimen from the individual with an ARP28 binding agent the selectively binds an ARP28 polypeptide; determining a test expression level of ARP28 polypeptide in the specimen; and comparing the test expression level to a non-neoplastic control expression level of ARP28 polypeptide, where an altered test expression level as compared to the control expression level indicates the presence of a prostate neoplastic condition in the individual. A specimen useful in the invention can include, for example, prostate tissue, or can be, for example, a blood, serum, urine or semen specimen. ARP28 binding agents useful in the methods of the invention include, but are not limited to, antibodies. In one embodiment, a method of the invention is practiced with an ARP28 binding agent that selectively binds at least eight contiguous amino acids of human ARP28 (SEQ ID NO: 20).

The invention also provides herein a method of diagnosing or predicting susceptibility to a prostate neoplastic condition in an individual by contacting a specimen from the individual with an ARP30 binding agent that selectively binds an ARP30 polypeptide; determining a test expression level of ARP30 polypeptide in the specimen; and comparing the test expression level to a non-neoplastic control expression level of ARP30 polypeptide, where an altered test expression level as compared to the control expression level indicates the presence of a prostate neoplastic condition in the individual. A specimen useful in the invention can include, for example, prostate tissue, or can be, for example, a blood, serum, urine or semen specimen. ARP30 binding agents useful in the methods of the invention include, but are not limited to, antibodies. Additional ARP30 binding agents useful in the invention include those that selectively bind at least eight contiguous amino acids of human ARP30 (SEQ ID NO: 22).

The invention also provides herein a method of diagnosing or predicting susceptibility to a prostate neoplastic condition in an individual by contacting a specimen from the individual with an ARP33 binding agent that selectively binds an ARP33 polypeptide; determining a test expression level of ARP33 polypeptide in the specimen; and comparing the test expression level to a non-neoplastic control expression level of ARP33 polypeptide, where an altered test expression level as compared to the control expression level indicates the presence of a prostate neoplastic condition in the individual. A specimen useful in the invention can include, for example, prostate tissue, or can be, for example, a blood, serum, urine or semen specimen. ARP33 binding agents useful in the methods of the invention encompass, without limitation, antibodies. In one embodiment, a method of the invention is practiced with an ARP33 binding agent that selectively binds human ARP33 (SEQ ID NO: 24). In another embodiment, a method of the invention is practiced with an ARP33 binding agent that selectively binds at least eight contiguous amino acids of residues 1-132 of SEQ ID NO: 24. In yet a further embodiment, a method of the invention is practiced with an ARP33 binding agent that selectively binds at least eight contiguous amino acids of residues 251-405 of SEQ ID NO: 24.

The present invention also provides a method of diagnosing or predicting susceptibility to a prostate neoplastic condition in an individual by contacting a specimen from the individual with an ARP11 binding agent that selectively binds an ARP11 polypeptide; determining a test expression level of ARP11 polypeptide in the specimen; and comparing the test expression level to a non-neoplastic control expression level of ARP11 polypeptide, where an altered test expression level as compared to the control expression level indicates the presence of a prostate neoplastic condition in the individual. The method can be practiced with, for example, a prostate tissue specimen, or with a specimen of blood, serum, urine or semen. In one embodiment, a method of the invention is practiced with an ARP11 binding agent which is an antibody that selectively binds at least eight contiguous amino acids of human ARP11 (SEQ ID NO: 34).

In a method of the invention, the specimen can contain, for example, a prostate cell or prostate tissue and, in one embodiment, is a fluid such as blood, serum, urine or semen. The control expression level can be determined, for example, using a normal prostate cell or an androgen-dependent cell line. In addition, a binding agent selective for a polypeptide of the invention can be, for example, an antibody, and, if desired, can further include a detectable label.

As used herein, the term “specimen” means any biological material including fluid, cell, tissue, organ or portion thereof, that contains or potentially contains an ARP polypeptide of the invention. The term specimen includes materials present in an individual as well as materials obtained or derived from the individual. For example, a specimen can be a histologic section obtained by biopsy, or cells that are placed in or adapted to tissue culture. A specimen further can be a subcellular fraction or extract, or a crude or substantially pure protein preparation. A specimen can be prepared by methods known in the art suitable for the particular format of the detection method.

In methods of the invention, the specimen can be, for example, a prostate cell or prostate tissue such as a tissue biopsy. A specimen can also be a fluid sample, for example, blood, serum, urine or semen. A normal specimen can be, for example, a normal prostate cell or an androgen-dependent cell line.

These diagnostic methods of the invention rely on a binding agent. As described above, the term “binding agent” when used in reference to an ARP polypeptide, is intended to mean a compound, including a simple or complex organic molecule, a metal containing compound, carbohydrate, peptide, protein, peptidomimetic, glycoprotein, lipoprotein, lipid, nucleic acid molecule, antibody, or the like that selectively binds the specified ARP polypeptide, or fragment thereof. The binding agent can be labeled with a detectable moiety, if desired, or rendered detectable by specific binding to a detectable secondary binding agent. Exemplary binding agents are discussed hereinabove.

A prostate neoplastic condition is a benign or malignant prostate lesion of proliferating cells. Prostate neoplastic conditions include, for example, prostate interepithelial neoplasia (PIN) and prostate cancer. Prostate cancer is an uncontrolled proliferation of prostate cells which can invade and destroy adjacent tissues as well as metastasize. Primary prostate tumors can be classified into stages TX, T0, T1, T2, T3, and T4 and metastatic tumors can be classified into stages D1, D2 and D3. Similarly, there are classifications known by those skilled in the art for the progressive stages of precancerous lesions or PIN. The methods herein are applicable for the diagnosis or treatment of any or all stages of prostate neoplastic conditions.

The methods of the invention are also applicable to prostate pathologies other than neoplastic conditions. Such other pathologies include, for example, benign prostatic hyperplasia (BPH) and prostatitis. BPH is one of the most common diseases in adult males. Histological evidence of BPH has been found in more than 40% of men in their fifties and almost 90% of men in their eighties. The disease results from the accumulation of non-malignant nodules arising in a small region around the proximal segment of the prostatic urethra which leads to an increase in prostate volume. If left untreated, BPH can result in acute and chronic retention of urine, renal failure secondary to obstructive uropathy, serious urinary tract infection and irreversible bladder decompensation. Prostatitis is an infection of the prostate. Other prostate pathologies known to those skilled in the art exist as well and are similarly applicable for diagnosis or treatment using the methods of the invention. Various neoplastic conditions of the prostate as well as prostate pathologies can be found described in, for example, Campbell's Urology, Seventh Edition, W.B. Saunders Company, Philadelphia (1998). Therefore, the methods of the invention are applicable to both prostate neoplastic conditions and prostate pathologies.

Therefore, the invention provides a method for both diagnosing and prognosing a prostate neoplastic condition including prostate cancer and prostate interepithelial neoplasia as well as other prostate pathologies such as BPH and prostatitis.

The invention provides a method of diagnosing or predicting prostate neoplastic conditions based on a finding of a positive correlation between a test expression level of an ARP polypeptide or nucleic acid in neoplastic cells of the prostate and the degree or extent of the neoplastic condition or pathology. The diagnostic methods of the invention are applicable to numerous prostate neoplastic conditions and pathologies as described above. One consequence of progression into these neoplastic and pathological conditions can be altered expression of ARP polypeptide or nucleic acid in prostate tissue. The alteration in ARP polypeptide or nucleic acid expression in individuals suffering from a prostate neoplastic condition can be measured by comparing the amount of ARP polypeptide or nucleic acid to that found, for example, in normal prostate tissue samples or in normal blood or serum samples. A two-fold or more increase or decrease in a test expression level in a prostate cell sample relative to a non-neoplastic control expression sample obtained, for example, from normal prostate cells or from an androgen-dependent cell line is indicative of a prostate neoplastic condition or pathology. Similarly, an alteration in ARP polypeptide or nucleic acid expression leading to an increased or decreased secretion into the blood or other circulatory fluids of the individual compared to a non-neoplastic control blood or fluid samples also can be indicative of a prostate neoplastic condition or pathology. For example, an alteration in ARP polypeptide or nucleic acid expression can lead to a two-fold, five-fold, ten-fold, 100-fold, 200-fold or 1000-fold increased secretion into the blood or other circulatory fluids of the individual compared to a non-neoplastic control blood or fluid samples. As another example, an alteration in ARP polypeptide or nucleic acid expression can lead to a two-fold, five-fold, ten-fold, 100-fold, 200-fold or 1000-fold decreased secretion into the blood or other circulatory fluids of the individual compared to a non-neoplastic control blood or fluid samples.

As a diagnostic indicator, an ARP polypeptide or nucleic acid molecule can be used qualitatively to positively identify a prostate neoplastic condition or pathology as described above. Alternatively, ARP polypeptide or nucleic acid molecule also can be used quantitatively to determine the degree or susceptibility of a prostate neoplastic condition or pathology. For example, successive increases or decreases in the expression levels of ARP polypeptide or nucleic acid can be used as a predictive indicator of the degree or severity of a prostate neoplastic condition or pathology. For example, increased expression can lead to a rise in accumulated levels and can be positively correlated with increased severity of a neoplastic condition of the prostate. A higher level of ARP polypeptide or nucleic acid expression can be correlated with a later stage of a prostate neoplastic condition or pathology. For example, increases in expression levels of two-fold or more compared to a normal sample can be indicative of at least prostate neoplasia. ARP polypeptide or nucleic acid molecule also can be used quantitatively to distinguish between pathologies and neoplastic conditions as well as to distinguish between the different types of neoplastic conditions.

Correlative alterations can be determined by comparison of ARP polypeptide or nucleic acid expression from the individual having, or suspected of having, a neoplastic condition of the prostate to expression levels of ARP polypeptide or nucleic acid from known specimens or samples determined to exhibit a prostate neoplastic condition. Alternatively, correlative alterations also can be determined by comparison of a test expression level of ARP polypeptide or nucleic acid expression to expression levels of other known markers of prostate cancer such as prostate specific antigen (PSA), glandular kallikrein 2 (hK2) and prostase/PRSS18. These other known markers can be used, for example, as an internal or external standard for correlation of stage-specific expression with altered ARP polypeptide or nucleic acid expression and severity of the neoplastic or pathological condition. Conversely, a regression in the severity of a prostate neoplastic condition or pathology can be followed by a corresponding reversal in ARP polypeptide or nucleic acid expression levels and can similarly be assessed using the methods described herein.

Given the teachings and guidance provided herein, those skilled in the art will know or can determine the stage or severity of a prostate neoplastic condition or pathology based on a determination of ARP polypeptide or nucleic acid expression and correlation with a prostate neoplastic condition or pathology. A correlation can be determined using known procedures and marker comparisons as described herein. For a review of recognized values for such other marker in normal versus pathological tissues, see, for example, Campbell's Urology, Seventh Edition, W.B. Saunders Company, Philadelphia (1998).

The use of ARP polypeptide or nucleic acid expression levels in prostate cells, the circulatory system and urine as a diagnostic indicator of a prostate pathology allows for early diagnosis as a predictive indicator when no physiological or pathological symptoms are apparent. The methods are particularly applicable to any males over age 50, African-American males and males with familial history of prostate neoplastic conditions or pathologies. The diagnostic methods of the invention also are particularly applicable to individuals predicted to be at risk for prostate neoplastic conditions or pathologies by reliable prognostic indicators prior to onset of overt clinical symptoms. All that is necessary is to determine the ARP polypeptide or nucleic acid prostate tissue or circulatory or bodily fluid expression levels to determine whether there is altered ARP polypeptide or nucleic acid levels in the individual suspected of having a prostate pathology compared to a control expression level such as the level observed in normal individuals. Those skilled in the art will know by using routine examinations and practices in the field of medicine those individuals who are applicable candidates for diagnosis by the methods of the invention.

For example, individuals suspected of having a prostate neoplastic condition or pathology can be identified by exhibiting presenting signs of prostate cancer which include, for example, a palpable nodule (>50% of the cases), dysuria, cystitis and prostatitis, frequency, urinary retention, or decreased urine stream. Signs of advanced disease include pain, uremia, weight loss and systemic bleeding. Prognostic methods of this invention are applicable to individuals after diagnosis of a prostate neoplastic condition, for example, to monitor improvements or identify residual neoplastic prostate cells using, for example, imaging methods known in the art and which target ARP polypeptide or nucleic acid. Therefore, the invention also provides a method of predicting the onset of a prostate neoplastic condition or pathology by determining an altered test expression level of one of the ARP nucleic acid molecules or polypeptides of the invention.

The diagnostic methods of the invention are applicable for use with a variety of different types of samples or specimens isolated or obtained from an individual having, or suspected of having a prostate neoplastic condition or prostate pathology. For example, samples applicable for use in one or more diagnostic formats of the invention include tissue and cell samples. A tissue or cell sample or specimen can be obtained, for example, by biopsy or surgery. As described below, and depending on the format of the method, the tissue can be used whole or subjected to various methods known in the art to disassociate the sample or specimen into smaller pieces, cell aggregates or individual cells. Additionally, when combined with amplification methods such as polymerase chain reaction (PCR), a single prostate cell can be a sample sufficient for use in diagnostic assays of the invention which employ hybridization detection methods. Similarly, when measuring ARP polypeptide or activity levels, amplification of the signal with enzymatic coupling or photometric enhancement can be employed using only a few or a small number of cells.

Whole tissue obtained from a prostate biopsy or surgery is one example of a prostate cell sample or specimen. Whole tissue prostate cell samples or specimens can be assayed employing any of the formats described below. For example, the prostate tissue sample can be mounted and hybridized in situ with ARP nucleic acid probes. Similar histological formats employing protein detection methods and in situ activity assays also can be used to detect an ARP polypeptide in whole tissue prostate cell specimens. Protein detection methods include, for example, staining with an ARP specific antibody and activity assays. Such histological methods as well as others well known to those skilled in the art are applicable for use in the diagnostic methods of the invention using whole tissue as the source of a prostate cell specimen. Methods for preparing and mounting the samples and specimens are similarly well known in the art.

Individual prostate cells and cell aggregates from an individual having, or suspected of having a prostate neoplastic condition or pathology also are prostate cell samples which can be analyzed for an altered test expression level in a method of the invention. The cells can be grown in culture and analyzed in situ using procedures such as those described above. Whole cell samples expressing cell surface markers associated with ARP polypeptide or nucleic acid expression can be rapidly tested using fluorescent or magnetic activated cell sorting (FACS or MACS) with labeled binding agents selective for the surface marker or using binding agents selective for epithelial or prostate cell populations, for example, and then determining a test expression level of a specified ARP polypeptide or nucleic acid within this population. The test expression level can be determined using, for example, binding agents selective for polypeptides of the invention or by hybridization to a specific nucleic acid molecule of the invention. Other methods for measuring the expression level of ARP polypeptide or nucleic acid in whole cell samples are known in the art and are similarly applicable in any of the diagnostic formats described below.

The tissue or whole cell prostate cell sample or specimen obtained from an individual also can be analyzed for increased ARP polypeptide or nucleic acid expression by lysing the cell and measuring a test expression levels of ARP polypeptide or nucleic acid in the lysate, a fractionated portion thereof or a purified component thereof using any of diagnostic formats described herein. For example, if a hybridization format is used, ARP RNA can be amplified directly from the lysate using PCR, or other amplification procedures well known in the art such as RT-PCR, 5′ or 3′ RACE to directly measure the expression levels of ARP nucleic acid molecules. RNA also can be isolated and probed directly such as by solution hybridization or indirectly by hybridization to immobilized RNA. Similarly, when determining a test expression level of ARP using polypeptide detection formats, lysates can be assayed directly, or they can be further fractionated to enrich for ARP polypeptide and its corresponding activity. Numerous other methods applicable for use with whole prostate cell samples are well known to those skilled in the art and can accordingly be used in the methods of the invention.

The prostate tissue or cell sample or specimen can be obtained directly from the individual or, alternatively, it can be obtained from other sources for testing. Similarly, a cell sample can be tested when it is freshly isolated or it can be tested following short or prolonged periods of cryopreservation without substantial loss in accuracy or sensitivity. If the sample is to be tested following an indeterminate period of time, it can be obtained and then cryopreserved, or stored at 4° C. for short periods of time, for example. An advantage of the diagnostic methods of the invention is that they do not require histological analysis of the sample. As such, the sample can be initially disaggregated, lysed, fractionated or purified and the active component stored for later diagnosis.

The diagnostic methods of the invention are applicable for use with a variety of different types of samples and specimens other than prostate cell samples. For example, an ARP polypeptide or fragment thereof that is released into the extracellular space, including circulatory fluids as well as other bodily fluids, can be detected in a method of the invention. In such a case, the diagnostic methods of the invention are practiced with fluid samples collected from an individual having, or suspected of having a neoplastic condition of the prostate or a prostate pathology.

Fluid samples and specimens, which can be measured for ARP polypeptide or nucleic acid expression levels, include, for example, blood, serum, lymph, urine and semen. Other bodily fluids are known to those skilled in the art and are similarly applicable for use as a sample or specimen in the diagnostic methods of the invention. One advantage of analyzing fluid samples or specimens is that they are readily obtainable, in sufficient quantity, without invasive procedures as required by biopsy and surgery. Analysis of fluid samples or specimens such as blood, serum and urine will generally be in the diagnostic formats described herein which measure ARP polypeptide levels or activity. As the ARP related polypeptide is circulating in a soluble form, the methods will be similar to those which measure expression levels from cell lysates, fractionated portions thereof or purified components.

Prostate neoplastic conditions and prostate pathologies can be diagnosed, predicted or prognosed by measuring a test expression level of ARP polypeptide or nucleic acid in a prostate cell sample, circulating fluid or other bodily fluid obtained from the individual. As described herein, a test or control expression level can be measured by a variety of methods known in the art. For example, a test expression level of a specified ARP can be determined by measuring the amount of ARP RNA or polypeptide in a sample or specimen from the individual. Alternatively, a test expression level of ARP can be determined by measuring the amount of an ARP activity in a specimen, the amount of activity being indicative of the specified ARP polypeptide expression level.

One skilled in the art can readily determine an appropriate assay system given the teachings and guidance provided herein and choose a method based on measuring ARP RNA, polypeptide or activity. Considerations such as the sample or specimen type, availability and amount will also influence selection of a particular diagnostic format. For example, if the sample or specimen is a prostate cell sample and there is only a small amount available, then diagnostic formats which measure the amount of ARP RNA by, for example, PCR amplification, or which measure ARP-related cell surface polypeptide by, for example, FACS analysis can be appropriate choices for determining a test expression level. Alternatively, if the specimen is a blood sample and the user is analysing numerous different samples simultaneous, such as in a clinical setting, then a multisample format, such as an Enzyme Linked Immunoabsorbant Assay (ELISA), which measures the amount of an ARP polypeptide can be an appropriate choice for determining a test expression level of a specified ARP. Additionally, ARP nucleic acid molecules released into bodily fluids from the neoplastic or pathological prostate cells can also be analyzed by, for example, PCR or RT-PCR. Those skilled in the art will know, or can determine which format is amenable for a particular application and which methods or modifications known within the art are compatible with a particular type of format.

Hybridization methods are applicable for measuring the amount of ARP RNA as an indicator of ARP expression levels. There are numerous methods well known in the art for detecting nucleic acid molecules by specific or selective hybridization with a complementary nucleic acid molecule. Such methods include both solution hybridization procedures and solid-phase hybridization procedures where the probe or sample is immobilized to a solid support. Descriptions for such methods can be found in, for example, Sambrook et al., supra, and in Ausubel et al., supra. Specific examples of such methods include PCR and other amplification methods such as RT-PCR, 5′ or 3′ RACE, RNase protection, RNA blot, dot blot or other membrane-based technologies, dip stick, pin, ELISA or two-dimensional arrays immobilized onto chips as a solid support. These methods can be performed using either qualitative or quantitative measurements, all of which are well known to those skilled in the art.

PCR or RT-PCR can be used with isolated RNA or crude cell lysate preparations. As described previously, PCR is advantageous when there is limiting amounts of starting material. A further description of PCR methods can be found in, for example, Dieffenbach, C. W., and Dveksler, G. S., PCR Primer: A Laboratory Manual, Cold Spring Harbor Press, Plainview, N.Y. (1995). Multisample formats such as an ELISA or two-dimensional array offer the advantage of analyzing numerous, different samples in a single assay. Solid-phase dip stick-based methods offer the advantage of being able to rapidly analyze a patient's fluid sample and obtain an immediate result.

Nucleic acid molecules useful for measuring a test expression level of a specified ARP RNA are disclosed herein above. Briefly, for detection by hybridization, an ARP nucleic acid molecule having a detectable label is added to a prostate cell sample or a fluid sample obtained from the individual having, or suspected of having a prostate neoplastic condition or pathology under conditions which allow annealing of the molecule to an ARP RNA. Methods for detecting ARP RNA in a sample can include the use of, for example, RT-PCR. Conditions are well known in the art for both solution and solid phase hybridization procedures. Moreover, optimization of hybridization conditions can be performed, if desired, by hybridization of an aliquot of the sample at different temperatures, durations and in different buffer conditions. Such procedures are routine and well known to those skilled in the art. Following annealing, the sample is washed and the signal is measured and compared with a suitable control or standard value. The magnitude of the hybridization signal is directly proportional to the expression levels of ARP RNA.

The diagnostic procedures described herein can additionally be used in conjunction with other prostate markers, such as prostate specific antigen, human glandular kallikrein 2 (hk2) and prostase/PRSS18 for simultaneous or independent corroboration of a sample. Additionally, ARP polypeptide or nucleic acid expression can be used, for example, in combination with other markers to further distinguish normal basal cells, secretory cells and neoplastic cells of the prostate. Moreover, ARP polypeptide or nucleic acid expression can be used in conjunction with smooth muscle cell markers to distinguish between pathological conditions such as benign prostate hypertrophy (BPH) and neoplasia. Those skilled in the art will know which markers are applicable for use in conjunction with ARP polypeptide or nucleic acid to delineate more specific diagnostic information such as that described above.

The invention also provides diagnostic methods based on determining whether there is an altered test expression level of an ARP16, ARP8, ARP9, ARP13, ARP20, ARP24, ARP30, ARP33 or ARP11 polypeptide using a binding agent that selectively binds at least eight contiguous amino acids of the recited polypeptide. Essentially all modes of affinity binding assays are applicable for use in determining a test expression level of an ARP polypeptide in a method of the invention. Such methods are rapid, efficient and sensitive. Moreover, affinity binding methods are simple and can be modified to be performed under a variety of clinical settings and conditions to suit a variety of particular needs. Affinity binding assays which are known and can be used in the methods of the invention include both soluble and solid phase formats. A specific example of a soluble phase affinity binding assay is immunoprecipitation using an ARP selective antibody or other binding agent. Solid phase formats are advantageous in that they are rapid and can be performed easily and simultaneously on multiple different samples without losing sensitivity or accuracy. Moreover, solid phase affinity binding assays are further amenable to high throughput and ultra high throughput screening and automation.

Specific examples of solid phase affinity binding assays include immunoaffinity binding assays such as an ELISA and radioimmune assay (RIA). Other solid phase affinity binding assays are known to those skilled in the art and are applicable to the methods of the invention. Although affinity binding assays are generally formatted for use with an antibody binding molecule that is selective for the analyte or ligand of interest, essentially any binding agent can be alternatively substituted for the selectively binding antibody. Such binding agents include, for example, macromolecules such as polypeptides, peptides, nucleic acid molecules, lipids and sugars as well as small molecule compounds. Methods are known in the art for identifying such molecules which bind selectively to a particular analyte or ligand and include, for example, surface display libraries and combinatorial libraries. Thus, for a molecule other than an antibody to be used in an affinity binding assay, all that is necessary is for the binding agent to exhibit selective binding activity for a polypeptide of the invention.

Various modes of affinity binding formats are similarly known which can be used in the diagnostic methods of the invention. For the purpose of illustration, particular embodiments of such affinity binding assays will be described further in reference to immunoaffinity binding assays. The various modes of affinity binding assays, such as immunoaffinity binding assays, include, for example, solid phase ELISA and RIA as well as modifications thereof. Such modifications thereof include, for example, capture assays and sandwich assays as well as the use of either mode in combination with a competition assay format. The choice of which mode or format of immunoaffinity binding assay to use will depend on the intent of the user. Such methods can be found described in common laboratory manuals such as Harlow and Lane, Using Antibodies: A Laboratory Manual, Cold Spring Harbor Laboratory Press, New York (1999).

As with the hybridization methods described previously, the diagnostic formats employing affinity binding can be used in conjunction with a variety of detection labels and systems known in the art to quantitate amounts of a polypeptide of the invention in the analyzed sample. Detection systems include the detection of bound polypeptide on the invention by both direct and indirect means. Direct detection methods include labeling of the ARP-selective antibody or binding agent. Indirect detection systems include, for example, the use of labeled secondary antibodies and binding agents.

Secondary antibodies, labels and detection systems are well known in the art and can be obtained commercially or by techniques well known in the art. The detectable labels and systems employed with the ARP-selective binding agent should not impair binding of the agent to the corresponding ARP polypeptide. Moreover, multiple antibody and label systems can be employed for detecting the bound ARP-selective antibody to enhance the sensitivity of the binding assay if desired.

As with the hybridization formats described previously, detectable labels can be essentially any label that can be quantitated or measured by analytical methods. Such labels include, for example, enzymes, radioisotopes, fluorochromes as well as chemi- and bioluminescent compounds. Specific examples of enzyme labels include horseradish peroxidase (HRP), alkaline phosphatase (AP), β-galactosidase, urease and luciferase.

A horseradish-peroxidase detection system can be used, for example, with the chromogenic substrate tetramethylbenzidine (TMB), which yields a soluble product in the presence of hydrogen peroxide that is detectable by measuring absorbance at 450 nm. An alkaline phosphatase detection system can be used with the chromogenic substrate p-nitrophenyl phosphate, for example, which yields a soluble product readily detectable by measuring absorbance at 405 nm. Similarly, a β-galactosidase detection system can be used with the chromogenic substrate o-nitrophenyl-β-D-galactopyranoside (ONPG), which yields a soluble product detectable by measuring absorbance at 410 nm, or a urease detection system can be used with a substrate such as urea-bromocresol purple (Sigma Immunochemicals, St. Louis, Mo.). Luciferin is the substrate compound for luciferase which emits light following ATP-dependent oxidation.

Fluorochrome detection labels are rendered detectable through the emission of light of ultraviolet or visible wavelength after excitation by light or another energy source. DAPI, fluorescein, Hoechst 33258, R-phycocyanin, B-phycoerythrin, R-phycoerythrin, rhodamine, Texas red and lissamine are specific examples of fluorochrome detection labels that can be utilized in the affinity binding formats of the invention. A particularly useful fluorochrome is fluorescein or rhodamine.

Chemiluminescent as well as bioluminescent detection labels are convenient for sensitive, non-radioactive detection of an ARP polypeptide and can be obtained commercially from various sources such as Amersham Lifesciences, Inc. (Arlington Heights, Ill.).

Alternatively, radioisotopes can be used as detectable labels in the methods of the invention. Iodine-125 is a specific example of a radioisotope useful as a detectable label.

Signals from detectable labels can be analyzed, for example, using a spectrophotometer to detect color from a chromogenic substrate; a fluorometer to detect fluorescence in the presence of light of a certain wavelength; or a radiation counter to detect radiation, such as a gamma counter for detection of iodine-125. For detection of an enzyme-linked secondary antibody, for example, a quantitative analysis of the amount of bound agent can be made using a spectrophotometer such as an EMAX Microplate Reader (Molecular Devices, Menlo Park, Calif.) in accordance with the manufacturer's instructions. If desired, the assays of the invention can be automated or performed robotically, and the signal from multiple samples can be detected simultaneously.

The diagnostic formats of the present invention can be forward, reverse or simultaneous as described in U.S. Pat. No. 4,376,110 and No. 4,778,751. Separation steps for the various assay formats described herein, including the removal of unbound secondary antibody, can be performed by methods known in the art (Harlow and Lane, supra). For example, washing with a suitable buffer can be followed by filtration, aspiration, vacuum or magnetic separation as well as by centrifugation.

A binding agent selective for an ARP polypeptide also can be utilized in imaging methods that are targeted at ARP expressing prostate cells. These imaging techniques have utility in identification of residual neoplastic cells at the primary site following standard treatments including, for example, radical prostatectomy, radiation or hormone therapy. In addition, imaging techniques that detect neoplastic prostate cells have utility in detecting secondary sites of metastasis. A binding agent that selectively binds an ARP16, ARP8, ARP9, ARP13, ARP20, ARP24, ARP30, ARP33 or ARP11 polypeptide can be radiolabeled with, for example, ¹¹¹indium and infused intravenously as described by Kahn et al., Journal of Urology 152:1952-1955 (1994). The binding agent selective for an ARP polypeptide can be, for example, a monoclonal antibody selective for an ARP polypeptide. Imaging can be accomplished by, for example, radioimmunoscintigraphy as described by Kahn et al., supra.

In one embodiment, the invention provides a method of diagnosing or predicting the susceptibility of a prostate neoplastic condition in an individual suspected of having a neoplastic condition of the prostate, where a test expression level of an ARP polypeptide is determined by measuring the amount of ARP16, ARP8, ARP9, ARP13, ARP20, ARP24, ARP30, ARP33 or ARP11 polypeptide activity. The method is practiced by contacting a specimen from the individual with an agent that functions to measure an activity associated with an ARP16, ARP8, ARP9, ARP13, ARP20, ARP24, ARP30, ARP33 or ARP11 polypeptide of the invention.

As with the hybridization and affinity binding formats described above, activity assays similarly can be performed using essentially identical methods and modes of analysis. Therefore, solution and solid phase modes, including multisample ELISA, RIA and two-dimensional array procedures are applicable for use in measuring an activity associated with an ARP polypeptide. The activity can be measured by, for example, incubating an agent that functions to measure an activity associated with an ARP polypeptide with the sample and determining the amount of product formed that corresponds to ARP16, ARP8, ARP9, ARP13, ARP20, ARP24, ARP3, ARP33 or ARP11 polypeptide activity. The amount of product formed will directly correlate with the ARP16, ARP8, ARP9, ARP13, ARP20, ARP24, ARP30, ARP33 or ARP11 polypeptide activity in the specimen and therefore, with the expression levels of the corresponding polypeptide of the invention in the specimen.

The invention further provides a method of identifying a compound that inhibits ARP16, ARP8, ARP9, ARP13, ARP20, ARP24, ARP30, ARP33 or ARP11 polypeptide activity. The method consists of contacting a specimen containing an ARP polypeptide and an agent that functions to measure an activity associated with an ARP polypeptide with a test compound under conditions that allow formation of a product that corresponds to an ARP polypeptide activity and measuring the amount of product formed, where a decrease in the amount of product formed in the presence of the test compound compared to the absence of the test compound indicates that the compound has ARP polypeptide inhibitory activity. Similarly, compounds that increase the activity of an ARP polypeptide also can be identified. A test compound added to a specimen containing an ARP polypeptide and an agent that functions to measure an activity associated with an ARP polypeptide which increases the amount of product formed compared to the absence of the test compound indicates that the compound increases the corresponding ARP polypeptide activity. Therefore, the invention provides a method of identifying compounds that modulate the activity of an ARP polypeptide. The ARP polypeptide containing specimen used for such a method can be serum, prostate tissue, a prostate cell population or a recombinant cell population expressing an ARP polypeptide.

Those compounds having inhibitory activity are considered as potential ARP polypeptide antagonists and further as potential therapeutic agents for treatment of neoplastic conditions of the prostate. Similarly, those compounds which increase an ARP polypeptide activity are considered as potential ARP polypeptide agonists and further as potential therapeutic agents for the treatment of neoplastic conditions of the prostate. Each of these classes of compounds is encompassed by the term ARP regulatory agent as defined herein.

Within the biological arts, the term “about” when used in reference to a particular activity or measurement is intended to refer to the referenced activity or measurement as being within a range of values encompassing the referenced value and within accepted standards of a credible assay within the art, or within accepted statistical variance of a credible assay within the art.

A reaction system for identifying a compound that inhibits or enhances an ARP polypeptide activity can be performed using essentially any source of ARP polypeptide activity. Such sources include, for example, a prostate cell sample, lysate or fractionated portion thereof; a bodily fluid such as blood, serum or urine from an individual with a prostate neoplastic condition; a recombinant cell or soluble recombinant source, and an in vitro translated source. The ARP polypeptide source is combined with an agent that functions to measure an activity associated with an ARP polypeptide as described above and incubated in the presence or absence of a test inhibitory compound. The amount of product that corresponds to an ARP polypeptide activity that is formed in the presence of the test compound is compared with that in the absence of the test compound. Those test compounds which inhibit product formation are considered to be ARP polypeptide inhibitors. For example, a test compound can inhibit product formation by at least 50%, 80%, 90%, 95%, 99%, 99.5% or 99.9%. Similarly, those compounds which increase product formation are considered to be ARP polypeptide enhancers or activators. For example, a test compound can increase product formation by at least two-fold, five-fold, ten-fold, 100-fold, 200-fold or 1000-fold. ARP polypeptide inhibitors and activators can then be subjected to further in vitro or in vivo testing to confirm that they inhibit an ARP polypeptide activity in cellular and animal models.

Suitable test compounds for the inhibition or enhancement assays can be any substance, molecule, compound, mixture of molecules or compounds, or any other composition which is suspected of being capable of inhibiting an ARP polypeptide activity in vivo or in vitro. The test compounds can be macromolecules, such as biological polymers, including proteins, polysaccharides and nucleic acid molecules. Sources of test compounds which can be screened for ARP polypeptide inhibitory activity include, for example, libraries of peptides, polypeptides, DNA, RNA and small organic compounds. The test compounds can be selected randomly and tested by the screening methods of the present invention. Test compounds are administered to the reaction system at a concentration in the range from about 1 pM to 1 mM.

Methods for producing pluralities of compounds to use in screening for compounds that modulate the activity of an ARP polypeptide, including chemical or biological molecules that are inhibitors or enhancers of an ARP activity such as simple or complex organic molecules, metal-containing compounds, carbohydrates, peptides, proteins, peptidomimetics, glycoproteins, lipoproteins, nucleic acid molecules, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources. Combinatorial libraries of molecules can be prepared using well known combinatorial chemistry methods (Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997)).

Therefore, the invention provides a method of identifying a compound that inhibits or enhances an ARP polypeptide activity where the sample further consists of a prostate cell lysate, a recombinant cell lysate expressing an ARP polypeptide, an in vitro translation lysate containing an ARP mRNA, a fraction of a prostate cell lysate, a fraction of a recombinant cell lysate expressing an ARP polypeptide, a fractionated sample of an in vitro translation lysate containing an ARP mRNA or an isolated ARP polypeptide. The method can be performed in single or multiple sample format.

In another embodiment, polypeptides of the invention can be used as vaccines to prophylactically treat individuals for the occurrence of a prostate neoplastic condition or pathology. Such vaccines can be used to induce B or T cell immune responses or both aspects of the individuals endogenous immune mechanisms. The mode of administration and formulations to induce either or both of these immune responses are well known to those skilled in the art. For example, polypeptides can be administered in many possible formulations, including pharmaceutically acceptable mediums. They can be administered alone or, for example, in the case of a peptide, the peptide can be conjugated to a carrier, such as KLH, in order to increase its immunogenicity. The vaccine can include or be administered in conjunction with an adjuvant, various of which are known to those skilled in the art. After initial immunization with the vaccine, further boosters can be provided if desired. Therefore, the vaccines are administered by conventional methods in dosages which are sufficient to elicit an immunological response, which can be easily determined by those skilled in the art. Alternatively, the vaccines can contain anti-idiotypic antibodies which are internal images of polypeptides of the invention. Methods of making, selecting and administering such anti-idiotype vaccines are well known in the art. See, for example, Eichmann, et al., CRC Critical Reviews in Immunology 7:193-227 (1987). In addition, the vaccines can contain an ARP nucleic acid molecule. Methods for using nucleic acid molecules such as DNA as vaccines are well known to those skilled in the art (see, for example, Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997)).

The invention additionally provides a method of treating or reducing the severity of a prostate neoplastic condition.

Also provided by the invention is a method for treating or reducing the severity of a prostate neoplastic condition in an individual by administering to the individual an ARP7, ARP15, ARP16, ARP8, ARP9, ARP13, ARP20, ARP24, ARP26, ARP28, ARP30, ARP33 or ARP11 regulatory agent.

The invention further provides a method for treating or reducing the severity of a prostate neoplastic condition in an individual by administering to the individual an ARP6, ARP11, ARP12, ARP18, ARP19, ARP21, ARP22 or ARP29 regulatory agent. A method of the invention can be practiced by administering to an individual having a prostate neoplastic condition or other pro static pathology an ARP7, ARP15, ARP16, ARP8, ARP9, ARP13, ARP20, ARP24, ARP26, ARP28, ARP30, ARP33 or ARP11 regulatory agent. A “regulatory agent” means an agent that inhibits or enhances a biological activity of the specified ARP polypeptide. Such an ARP regulatory agent can effect the amount of ARP polypeptide produced or can inhibit or enhance activity without effecting the amount of polypeptide. Such an ARP regulatory agent can be, for example, a dominant negative form of ARP16, ARP8, ARP9, ARP13, ARP20, ARP24, ARP30, ARP33 or ARP11 polypeptide; an ARP16, ARP8, ARP9, ARP13, ARP20, ARP24, ARP30, ARP33 or ARP11 selective binding agent, or an ARP7, ARP15, ARP16, ARP8, ARP9, ARP13, ARP20, ARP24, ARP26, ARP28, ARP30, ARP33 or ARP11 antisense molecule. One skilled in the art understands that such an ARP7, ARP15, ARP16, ARP8, ARP9, ARP13, ARP20, ARP24, ARP26, ARP28, ARP30, ARP33 or ARP11 regulatory agent can be an agent that selectively regulates a biological activity of the specified ARP polypeptide or, alternatively, can be a non-selective agent that, in addition to regulating a biological activity of the specified polypeptide, also regulates the activity of one or more polypeptides.

AARP regulatory agent can cause a two-fold, five-fold, ten-fold, 20-fold, 100-fold or more reduction in the amount or activity of an ARP polypeptide. As another example, a regulatory agent can cause a two-fold, five-fold, ten-fold, 20-fold, 100-fold or more increase in the amount or activity of an ARP polypeptide or nucleic acid. ARP regulatory agents include ARP nucleic acid molecules, for example, antisense nucleic acid molecules; other nucleic acid molecules such as ribozymes; binding agents including antibodies, and compounds identified by the methods described herein. Such regulatory agents can be useful as therapeutics for treating or reducing the severity of an individual with a prostate neoplastic condition or for treating another pathology of the prostate.

One type of ARP regulatory agent is an inhibitor, means an agent effecting a decrease in the extent, amount or rate of ARP polypeptide expression or activity. An example of an ARP inhibitor is an ARP antisense nucleic acid molecule or a transcriptional inhibitor that binds to an ARP5′ promoter/regulatory region.

The term inhibitory amount means the amount of an inhibitor necessary to effect a reduction in the extent, amount or rate of ARP polypeptide. For example, an inhibitory amount of inhibitor can cause a two-fold, five-fold, ten-fold, 20-fold, 100-fold or more reduction in the amount or activity of an ARP polypeptide of the invention.

Such inhibitors can be produced using methods which are generally known in the art, and include the use of a purified ARP polypeptide to produce antibodies or to screen libraries of compounds, as described previously, for those which specifically bind a corresponding ARP polypeptide. For example, in one aspect, antibodies which are selective for an ARP polypeptide of the invention can be used directly as an antagonist, or indirectly as a targeting or delivery mechanism for bringing a cytotoxic or cytostatic agent to neoplastic prostate cells. Such agents can be, for example, radioisotopes. The antibodies can be generated using methods that are well known in the art and include, for example, polyclonal, monoclonal, chimeric, humanized single chain, Fab fragments, and fragments produced by a Fab expression library.

In another embodiment of the invention, ARP polynucleotides, or any fragment thereof, or antisense molecules, can be used as an ARP regulatory agent in a method of the invention. In one aspect, antisense molecules to an ARP encoding nucleic acid molecules can be used to block the transcription or translation of the corresponding mRNA. Specifically, cells can be transformed with sequences complementary to a nucleic acid molecule of the invention. Such methods are well known in the art, and sense or antisense oligonucleotides or larger fragments, can be designed from various locations along the coding or control regions of sequences encoding ARP polypeptides or nucleic acids. Thus, antisense molecules may be used to modulate an ARP activity, or to achieve regulation of an ARP gene function.

Expression vectors derived from retroviruses, adenovirus, adeno-associated virus (AAV), herpes or vaccinia viruses, or from various bacterial plasmids can be used for delivery of antisense nucleotide sequences to the prostate cell population. The viral vector selected should be able to infect the tumor cells and be safe to the host and cause minimal cell transformation. Retroviral vectors and adenoviruses offer an efficient, useful, and presently the best-characterized means of introducing and expressing foreign genes efficiently in mammalian cells. These vectors are well known in the art and have very broad host and cell type ranges, express genes stably and efficiently. Methods which are well known to those skilled in the art can be used to construct such recombinant vectors and are described in Sambrook et al., supra. Even in the absence of integration into the DNA, such vectors can continue to transcribe RNA molecules until they are disabled by endogenous nucleases. Transient expression can last for a month or more with a non-replicating vector and even longer if appropriate replication elements are part of the vector system.

Ribozymes, which are enzymatic RNA molecules, can also be used to catalyze the specific cleavage of an ARP mRNA. The mechanism of ribozyme action involves sequence-specific hybridization of the ribozyme molecule to complementary target ARP RNA, followed by endonucleolytic cleavage. Specific ribozyme cleavage sites within any potential RNA target are identified by scanning an ARP RNA for ribozyme cleavage sites which include the following sequences: GUA, GUU, and GUC. Once identified, short RNA sequences of between 15 and 20 ribonucleotides corresponding to the region of the target gene containing the cleavage site can be evaluated for secondary structural features which can render the oligonucleotide inoperable. The suitability of candidate targets can also be evaluated by testing accessibility to hybridization with complementary oligonucleotides using ribonuclease protection assays. Antisense molecules and ribozymes of the invention can be prepared by any method known in the art for the synthesis of nucleic acid molecules.

In another embodiment, an ARP promoter and regulatory region can be used for constructing vectors for prostate cancer gene therapy. The promoter and regulatory region can be fused to a therapeutic gene for prostate specific expression. This method can include the addition of one or more enhancer elements which amplify expression of the heterologous therapeutic gene without compromising tissue specificity. Methods for identifying a gene promoter and regulatory region are well known to those skilled in the art, for example, by selecting an appropriate primer from the 5′ end of the coding sequence and isolating the promoter and regulatory region from genomic DNA.

Examples of therapeutic genes that are candidates for prostate gene therapy utilizing an ARP promoter include suicide genes. The expression of suicide genes produces a protein or agent that directly or indirectly inhibits neoplastic prostate cell growth or promotes neoplastic prostate cell death. Suicide genes include genes encoding enzymes, oncogenes, tumor suppressor genes, genes encoding toxins, genes encoding cytokines, or a gene encoding oncostatin. The therapeutic gene can be expressed using the vectors described previously for antisense expression.

In accordance with another embodiment of the present invention, there are provided diagnostic systems, for example, in kit form. Such a diagnostic system contains at least one nucleic acid molecule or antibody of the invention in a suitable packaging material. The diagnostic kits containing nucleic acid molecules are derived from ARP nucleic acid molecules described herein. A diagnostic system of the invention can be useful for assaying for the presence or absence of an ARP nucleic acid molecule in either genomic DNA or mRNA.

A suitable diagnostic system includes at least one ARP nucleic acid molecule or antibody, as a separately packaged chemical reagent(s) in an amount sufficient for at least one assay. For a diagnostic kit containing a nucleic acid molecule of the invention, the kit will generally contain two or more nucleic acid molecules. When the diagnostic kit is to be used in PCR, the kit can further contain at least two oligonucleotides that can serve as primers for PCR. Those of skill in the art can readily incorporate nucleic acid molecules antibodies of the invention into kit form in combination with appropriate buffers and solutions for the practice of the invention methods as described herein. A kit containing an ARP polypeptide-specific antibody can contain a reaction cocktail that provides the proper conditions for performing an assay, for example, an ELISA or other immunoassay, for determining the level of expression of a corresponding ARP polypeptide in a specimen, and can contain control samples that contain known amounts of a corresponding ARP polypeptide and, if desired, a second antibody selective for the corresponding anti-ARP antibody.

The contents of the kit of the invention, for example, ARP nucleic acid molecules or antibodies, are contained in packaging material, which can provide a sterile, contaminant-free environment. In addition, the packaging material contains instructions indicating how the materials within the kit can be employed both to detect the presence or absence of a particular nucleic acid sequence or polypeptide of the invention or to diagnose the presence of, or a predisposition for a condition associated with the presence or absence of a nucleic acid sequence or polypeptide of the invention such as prostate cancer. The instructions for use typically include a tangible expression describing the reagent concentration or at least one assay method parameter, such as the relative amounts of reagent and sample to be admixed, maintenance time periods for reagent/sample admixtures, temperature, buffer conditions, and the like.

All journal article, reference, and patent citations provided above, in parentheses or otherwise, whether previously stated or not, are incorporated herein by reference.

It is understood that modifications which do not substantially affect the activity of the various embodiments of this invention are also included within the definition of the invention provided herein. Accordingly, the following examples are intended to illustrate but not limit the present invention.

### EXAMPLE I

### Isolation of ARP cDNAs

This example describes the isolation of several androgen-regulated sequences.

The ARP7 cDNA was identified as an androgen upregulated sequence as described below. The ARP7 (SEQ ID NO: 1) contains 5470 nucleotides. Nucleotides 474 to 4967 encode a polypeptide of 1498 amino acids (SEQ ID NO: 2). As shown in FIG. 1, ARP7 is dramatically up-regulated by androgen in starved LNCaP cells. As further shown in FIG. 2, ARP7 is most highly expressed in the prostate with little or no detectable expression in other tissues.

The human ARP15 cDNA (SEQ ID NO: 3), which contains 3070 nucleotides, has an open reading frame from nucleotide 253 to 1527 of SEQ ID NO: 3. The ARP15 cDNA sequence is predicted to encode a polypeptide of 425 amino acids (SEQ ID NO: 4) with at least three transmembrane domains (see Table 1). As shown in FIG. 3, ARP15 is expressed in prostate tissue and also expressed in testis and ovary.

The human ARP16 cDNA, shown herein as SEQ ID NO: 5, is a sequence of 2161 nucleotides with an open reading frame from nucleotide 138 to 1601. Furthermore, the human ARP16 is a polypeptide of 488 amino acids (SEQ ID NO: 4) with at least eight predicted transmembrane domains. As shown in FIG. 1, ARP16 mRNA is dramatically up-regulated by androgen in starved LNCaP cells.

ARP8 also was identified as a human sequence up-regulated by androgen in prostate cells. The human ARP8 cDNA (SEQ ID NO: 7) contains 2096 nucleotides with an open reading frame from nucleotides 1 to 1728; the encoded human ARP8 polypeptide (SEQ ID NO: 8) has 576 amino acids. The nucleic acid sequence of another human androgen-regulated cDNA expressed in prostate, ARP9 (SEQ ID NO: 9), was identified as described below. The ARP9 nucleic acid sequence disclosed herein has 2568 nucleotides with an open reading frame from nucleotide 559 to 2232. The encoded human ARP9 polypeptide (SEQ ID NO: 10) has 558 residues and is predicted to include at least four transmembrane domains. The ARP13 cDNA also increased in response to androgen in the LNCaP cell line. The ARP13 nucleotide sequence (SEQ ID NO: 11) has 2920 nucleotides with an open reading frame from nucleotide 141 to 1022. The human ARP13 polypeptide has the 294 amino acid sequence shown herein as SEQ ID NO: 12 and is predicted to include at least one transmembrane domain. The ARP20 nucleotide sequence shown herein as SEQ ID NO: 13 also was identified as positively regulated in response to androgen in LNCaP cells. The human ARP20 nucleotide sequence has 1095 nucleotides with an open reading frame from nucleotides 113 to 661; the human ARP20 polypeptide is shown herein as SEQ ID NO: 14.

ARP24, ARP26, ARP28, ARP30, ARP33 and ARP11 also were identified as androgen upregulated cDNAs expressed in the LnCaP prostate cell line. The ARP24 cDNA sequence shown herein as SEQ ID NO: 15 contains 3007 nucleotides with an open reading frame from nucleotides 38 to 1378; the encoded human ARP24 polypeptide has a 447 amino acid sequence (SEQ ID NO: 16) that is predicted to encode at least four transmembrane domains. The ARP26 cDNA sequence shown herein as SEQ ID NO: 17 was identified as a sequence of 3937 nucleotides with an open reading frame from nucleotides 240 to 1013. The corresponding androgen-regulated human ARP26 polypeptide (SEQ ID NO: 18) has 258 residues. Furthermore, the ARP28 cDNA sequence, shown herein as the 1401 nucleotide sequence SEQ ID NO: 19, contains an open reading frame from nucleotides 45 to 1085, which is predicted to encode the 347 amino acid human ARP28 polypeptide (SEQ ID NO: 20) with at least three transmembrane domains. The androgen-regulated ARP30 cDNA has a sequence (SEQ ID NO: 21) of 3318 nucleotides; the human ARP30 polypeptide (SEQ ID NO: 22), a protein of 601 amino acids, is encoded by an open reading frame positioned between nucleotides 252 to 2054 of SEQ ID NO: 21. Furthermore, the androgen-regulated ARP33 cDNA has a nucleic acid sequence (SEQ ID NO: 23) of 1690 nucleotides with an open reading frame from nucleotide 98 to 1313. The human ARP33 polypeptide, a protein of 405 residues shown herein as SEQ ID NO: 24, is predicted to include at least one transmembrane domain. The androgen-regulated ARP11 cDNA has a nucleic acid sequence (SEQ ID NO: 33) of 3067 nucleotides. An open reading frame from nucleotide 790 to 1805 encodes a protein of 338 residues (SEQ ID NO: 34). ARP6, ARP10, ARP12, ARP18, ARP19, ARP21, ARP22 and ARP29 also are androgen-regulated sequences expressed in prostate. The human ARP6 cDNA sequence is shown herein as a 504 nucleotide sequence (SEQ ID NO: 25); the human ARP11 cDNA sequence is shown herein as a 2189 nucleotide sequence (SEQ ID NO: 26); the human ARP12 cDNA sequence is shown herein as a 2576 nucleotide sequence (SEQ ID NO: 27); and the human ARP18 cDNA sequence is shown herein as a 521 nucleotide sequence (SEQ ID NO: 28). Furthermore, the human ARP19 cDNA sequence is shown herein as a 644 nucleotide sequence (SEQ ID NO: 29); the human ARP21 cDNA sequence is shown herein as a 1460 nucleotide sequence (SEQ ID NO: 30); the human ARP22 cDNA sequence is shown herein as a 774 nucleotide sequence (SEQ ID NO: 31); and the human ARP29 cDNA sequence is shown herein as a 386 nucleotide sequence (SEQ ID NO: 32).

TABLE 1
Summary of Transmembrane Domains Identified in ARPs

| Gene Name | TMPRED* |
|---|---|
| ARP 7 | 3 TMs** |
| ARP 15 | 3 TMs |
| ARP 16 | 8 TMs |
| ARP 8 | 0 |
| ARP 9 | 4 TMs |
| ARP 13 | 1 TM |
| ARP 24 | 4 TMs |
| ARP 28 | 3 TMs |
| ARP 30 | 0 |
| ARP 33 | 1 TM |

*TMPRED program at http://.ch.embnet.org/software/MPRED_form.html is used.
**Either CDS or the largest ORF is used for prediction, so the number of transmembranes (Tms) may be underestimated. Only scores above 500 are considered significant and reported here.

Cells were cultured as follows. LNCaP cells were cultured in RPMI 1640 medium with 5% FBS (Gibco-BRL). For androgen stimulation, six flasks (175 cm²) of LNCaP cells were starved for androgens by culturing in CS media (RPMI 1640 with 10% charcoal filtered FBS). After 48 hours of incubation, three flasks were incubated with CS media plus cycloheximide (1 μg/μl) and the other three were incubated with CS media plus 1 nM of R1881 and cycloheximide (1 μg/μl). All LNCaP cells were incubated for an additional 48 hours and then harvested. For time course experiments, LNCaP cells were harvested 4, 8, 12, 16, 24, 26, and 48 hours after incubation with R1881 containing media.

Microarray fabrication was performed essentially as follows. The 40 k sequence-verified cDNAs from Research Genetics, Inc., (Huntsville, Ala.) were PCR amplified according to the manufacturer's protocol. PCR products were purified in a 384-well format using MultiScreen PCR clean-up plates (Millipore, Bedford, Mass.) and verified by agarose gel electrophoresis. PCR products were re-suspended in a 384-well format at a concentration of 0.15 μg/μl in 3×SSC. After arraying the PCR products onto Type VII glass slides (Amersham) at 60% relative humidity and 20° C. using a 48-pin printhead on the ChipWriter high-speed robotics system (Virtek; Ontario, Calif.), arrayed slides were baked at 85° C. for two hours and then stored in a dessicator prior to use.

cDNA labeling and hybridization were performed essentially as follows. mRNA (1 μg) or total RNA (30 μg) was mixed with 1 μl of anchored oligo dT primer (Amersham), incubated at 70° C. for 10 minutes, and then chilled on ice. Then 4 μl of 5× first strand cDNA synthesis buffer (Gibco-BRL), 2 μl of 0.1 M DTT (Gibco-BRL), 1 μl of HPRI (20 μg/μl) (Amersham), and 1 μl of dNTP mix (Amersham); containing 2 mM dATP, 2 mM dGTP, 2 mM dTTP and 1 mM dCTP), 1 μl of Cy3 dCTP (1 mM) (Amersham) and 1 μl of SuperScript II RT (200 μg/μl) were added, and the mixture incubated at 42° C. for 2 hours. After first strand cDNA labeling, the reaction mixture was incubated at 94° C. for 3 minutes. Unlabeled RNAs were hybrolyzed by addition of 1 μl of 5N NaOH and incubation at 37° C. for 10 minutes. Subsequently, 1 μl of 5M HCl and 5 μl of 1M Tris-HCl (pH 7.5) were added to neutralize the reaction mixture. The mixture was then purified using a Qiagen PCR purification kit (Qiagen) essentially according to the manufacturer's protocol with two washes with PE buffer; DNA was eluted with 30 μl of dH₂O. The probe was mixed with 1 μl of dA/dT (12-18) (1 μg/μl) (Pharmacia) and 1 μl of human Cot I DNA (1 μg/μl) (Gibco-BRL) denatured at 94° C. for 5 minutes. An equal volume of 2× Microarray Hybridization Solution (Amersham) was added, and the mixture was prehybridized at 50° C. for 1 hour. After prehybridization, the probe mixture was added to an arrayed slide and covered with a cover slide. Hybridization was performed in a humid chamber at 52° C. for 16 hours. After hybridization, the slide was washed once with 1×SSC/0.2% SDS at room temperature for 5 minutes on a shaker, twice with 0.1×SSC/0.2% SDS at room temperature for 10 minutes, and once with 0.1×SSC at room temperature for 10 minutes. After washing, the slide was rinsed in distilled water to remove trace salts and dried. Hybridized microarray slides were scanned with the ScanArray 5000 (GSI Lumonics) at 10 um resolution.

Hybridization was repeated three times. For the first two hybridizations, RNAs from androgen-stimulated cells were labeled with Cy5 dCTP while RNAs from androgen-starved cells were labeled with Cy3 dCTP. For the third hybridization, RNAs from androgen-stimulated cells were labeled with Cy3 while RNAs from andorgen-starved cells were labeled with Cy5.

Microarray Data Analysis was performed as follows. Each spot on microarray was quantified with the QuantArray software (GSI Lumonics). Data were normalized with the median for each of the four duplicates. Statistical analyses were done using the software VERA and SAM. A lambda value, that describes how likely the gene is differentially expressed, was obtained for each spot on the array.

Northern hybridization was performed as follows. Total RNA (ten μg) was fractionated on 1.2% agarose denaturing gels and transferred to nylon membranes by capillary method (Maniatis). Human and mouse multiple tissue and master blots were purchased from CLONTECH. Blots were hybridized with DNA probes labeled with [alpha-³²P]dCTP by random priming using the Rediprime II random primer labeling system (Amersham) according to the manufacturer's protocol. Filters were imaged and quantitated using a phosphor-capture screen and Imagequant software (Molecular Dynamics).

### EXAMPLE II

### Characterization of ARP15

This example describes preparation of anti-ARP15 antibodies and characterization of ARP15 polypeptide expression.

### ARP15 is Expressed in Patient Serum

The coding region of the full-length ARP15 cDNA was cloned into PGEX 4T-1 (Pharmacia). The resulting GST-ARP15 fusion protein was expressed and purified according to the manufacturer's protocols (Pharmacia Inc.) The GST-ARP15 fusion protein was used to immunize mice using a standard protocol. Hybridomas were generated by standard methods and screened by differential ELISA using GST-ARP15 and GST proteins.

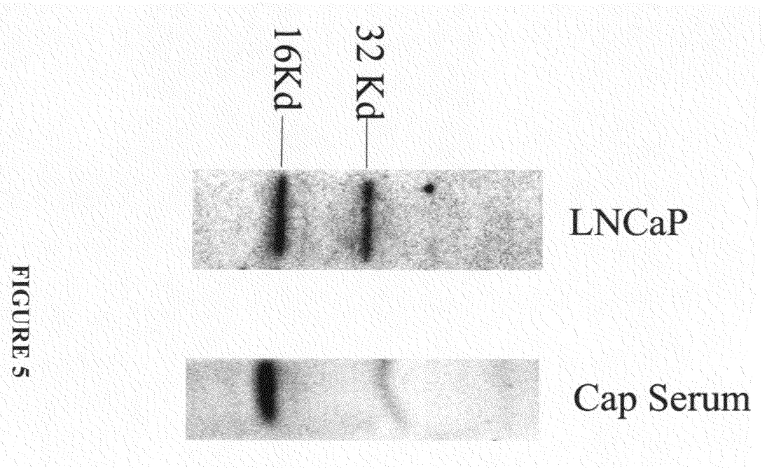

Monoclonal hydridomas were generated by limited dilution and screening using ELISA and Western blotting. Several clones were obtained that produced monoclonal antibodies: three clones secreted mAb of IgG1 isotype and one clone secreted mAb of IgG2b isotype. As shown in FIG. 5, monoclonal antibody “1R” detected bands of 32 kd and 16 kd both in a lysate prepared from the LNCaP cell line and in a serum sample from a prostate cancer patient.

### Cellular Localization of ARP15

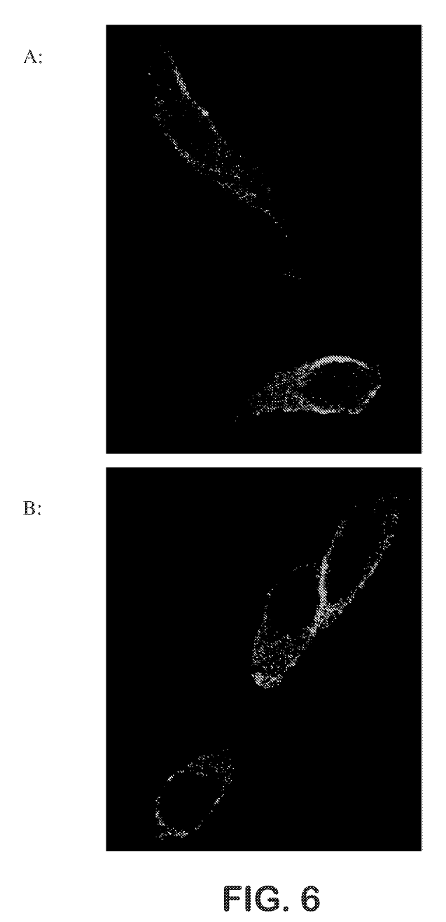

Using the anti-ARP15 monoclonal antibody “1R” prepared as described above, cell staining was performed. As shown in FIG. 6A, ARP15 was localized to the cell plasma membrane, similar to the expression pattern of β-integrin shown in FIG. 6B.

### Expression of ARP15 in Normal and Cancer Tissues

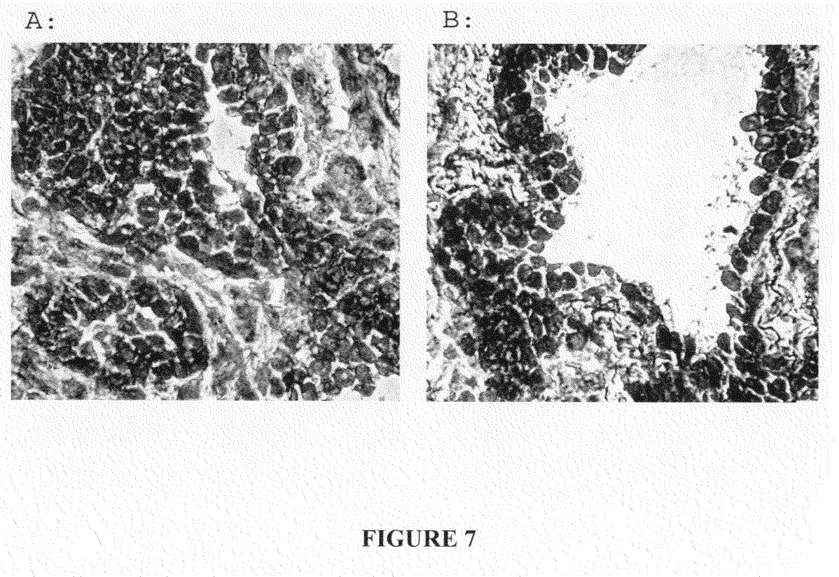

Immunohistochemical staining was performed using anti-ARP15 monoclonal antibody 1R against cancerous and normal prostate tissue sections. The immunostaining revealed that ARP15 protein expression was limited to prostate epithelial cells, with little or no expression in stromal cells (see FIG. 7). These results are consistent with the Northern analysis showing that ARP15 RNA is predominantly expressed in prostate, testis and ovary tissues.

1. A substantially pure ARP7 nucleic acid molecule, comprising the nucleotide sequence shown as SEQ ID NO: 1.

2. (canceled)

3. A method of diagnosing or predicting susceptibility to a prostate neoplastic condition in an individual, comprising:
(a) contacting a sample from said individual with an ARP7 nucleic acid molecule comprising at least 10 contiguous nucleotides of the nucleotide sequence of claim 1;
(b) determining a test expression level of ARP7 RNA in said sample; and
(c) comparing said test expression level to a non-neoplastic control expression level of ARP7 RNA,
wherein an altered test expression level as compared to said control expression level indicates the presence of a prostate neoplastic condition in said individual.

4. The method of claim 3, wherein said sample comprises prostate tissue.

5. The method of claim 3, wherein said sample is selected from the group consisting of blood, urine and semen.

6. The method of claim 3, wherein said ARP7 nucleic acid molecule is 15 to 35 nucleotides in length.

7-22. (canceled)

23. A substantially pure ARP16 nucleic acid molecule, comprising a nucleic acid sequence encoding an ARP16 polypeptide having at least 90% amino acid identity with SEQ ID NO: 6.

24. The substantially pure ARP16 nucleic acid molecule of claim 23, which encodes the amino acid sequence shown as SEQ ID NO:6.

25. The substantially pure ARP16 nucleic acid molecule of claim 24, comprising the nucleotide sequence shown as SEQ ID NO:5.

26. (canceled)

27. A method of diagnosing or predicting susceptibility to a prostate neoplastic condition in an individual, comprising:
(a) contacting a sample from said individual with an ARP16 nucleic acid molecule comprising at least 10 contiguous nucleotides of the nucleotide sequence of claim 25;
(b) determining a test expression level of ARP16 RNA in said sample; and
(c) comparing said test expression level to a non-neoplastic control expression level of ARP16 RNA,
wherein an altered test expression level as compared to said control expression level indicates the presence of a prostate neoplastic condition in said individual.

28. The method of claim 27, wherein said sample comprises prostate tissue.

29. The method of claim 27, wherein said sample is selected from the group consisting of blood, urine and semen.

30. The method of claim 27, wherein said ARP16 nucleic acid molecule is 15 to 35 nucleotides in length.

31-42. (canceled)

43. A substantially pure ARP8 nucleic acid molecule, comprising a nucleic acid sequence encoding an ARP8 polypeptide having at least 65% amino acid identity with SEQ ID NO: 8.

44. The substantially pure ARP8 nucleic acid molecule of claim 43, which encodes the amino acid sequence shown as SEQ ID NO: 8.

45. The substantially pure ARP8 nucleic acid molecule of claim 44, comprising the nucleotide sequence shown as SEQ ID NO: 7.

46. (canceled)

47. A method of diagnosing or predicting susceptibility to a prostate neoplastic condition in an individual, comprising:
(a) contacting a sample from said individual with an ARP8 nucleic acid molecule comprising at least 10 contiguous nucleotides of of the nucleotide sequence of claim 44;
(b) determining a test expression level of ARP8 RNA in said sample; and
(c) comparing said test expression level to a non-neoplastic control expression level of ARP8 RNA,
wherein an altered test expression level as compared to said control expression level indicates the presence of a prostate neoplastic condition in said individual.

48. The method of claim 47, wherein said sample comprises prostate tissue.

49. The method of claim 47, wherein said sample is selected from the group consisting of blood, urine and semen.

50. The method of claim 47, wherein said ARP8 nucleic acid molecule is 15 to 35 nucleotides in length.

51-241. (canceled)


SEQUENCE LISTING (34 sequences)

SEQ ID NO: 1 - DNA, 5470 bp, Homo sapiens
  CDS (474)...(4967)
  misc_feature (0)...(0): ARP7
cggccgccagtgtgctggaattcgcccttactcactatagggctcgagcggccgcccgggcaggtctcgccggaggagctgggccctgaatcaccctgctccccggccggctgtcggcgctgggggagggggtcccgggggtcgactcaccgatctgcccgatgaactcgatcttgatgccctggtgctccagccgcttgttggggttcttgagggcaaggctcaccttcccggagaccgtctccccgtcgtagaagaggaaatatttctccttcttcccgtcctccgtcttgtgctcggcccgcttcctactctctgcatcgttcagaaggatttccacctccacgctctgcccgaagccgaagaagctcatcgcaccgccgggccgggcgggtctcggaacgactcggcgcgcgcgcgggagcgagctttgaaagttgagcacggcggcggcgagccggtgccctgggatcatggtggcgttgcggggccttggtagcggcctgcagccctggtgtccgctggatcttagactcgaatgggttgacacagtgtgggaactggatttcacagagactgagcctttggatcccagcatagaagcagagatcatagagactggattggctgcattcacaaaactctatgaaagccttttaccctttgctactggagaacatggatctatggagagtatctggaccttcttcattgagaacaatgtttcccatagtacactggtggcattgttctatcattttgttcaaatagttcataagaagaatgtcagtgtacagtatcgagaatatggccttcatgccgctgggctttactttttgctactagaagtaccaggcagtgtagccaatcaagtattccacccagtgatgtttgacaaatgcattcagactctaaagaagagctggccccaggaatctaacttgaatcggaaaagaaagaaagaacagcctaagagctctcaggctaaccccgggaggcatagaaaaaggggaaagccacccaggagagaagatattgagatggatgaaattatagaagaacaagaagatgagaatatttgtttttctgcccgggacctttctcaaattcgaaatgccatctttcaccttttaaagaattttttaaggcttctgccaaagttttccttgaaagaaaagccacaatgtgtacagaattgtatagaggtctttgtttcattaactaattttgagccagttcttcatgaatgtcatgttacacaagccagagctcttaaccaagcaaaatacataccagaactggcttattatggattgtatttgctgtgctctcccattcatggagaaggagataaggtcatcagttgtgttttccatcaaatgctcagtgtaatattaatgttagaagttggtgaaggatcccatcgtgccccccttgctgttacctcccaagtcatcaactgtagaaaccaggcggtccagtttatcagcgcccttgtggatgaattaaaggagagtatattcccagtcgtccgtatcttactgcagcacatctgtgccaaggtggtagataaatcagagtatcgtacttttgcagcccagtccctagtccagctgctcagtaaacttccttgtggggaatacgctatgttcattgcctggctttacaaatactcccgaagttccaagatcccacaccgggtttttactcttgatgttgtcttagctctgttagaactgcctgaaagagaggtggataacaccctctccttggagcatcagaagttcttaaagcataagttcctggtgcaggaaattatgtttgatcgttgcttagacaaggcgcctactgtccgcagcaaggcactgtccagctttgcacactgtctggagttgactgttaccagtgcgtcggagagtatcctggagctcctgattaacagtcctacgttttctgtaatagagagtcaccctggtaccttactgagaaattcatcagctttttcctaccaaaggcagacatctaaccgttccgaaccctcaggggagatcaacatagacagcagtggtgaaacagttggatctggagaaagatgtgtcatggcaatgctgagaaggaggatcagggatgagaagaccaacgttaggaagtctgcactgcaggtattagtgagtattctgaaacactgtgatgtctcaggcatgaaggaagacctgtggattctgcaggaccagtgtcgggaccctgcagtgtctgtccggaagcaggccctccagtctcttactgaactccttatggctcagcctagatgcgtgcagatccagaaagcctggttgcggggggtggtcccggtggtgatggactgcgagagcactgtgcaggagaaggccctggagttcctggaccagctgctgctgcagaacatccggcatcacagtcattttcactctggggacgacagccaggtcctcgcctgggcgcttcttactctcctcaccaccgaaagccaggaactgagccgatatttaaataaggcttttcatatctggtccaagaaagaaaaattctcacccacttttataaacaatgtaatatctcacactggcacggaacattcggcacctgcctggatgctgctctccaagattgctggctcctcacccaggctggactacagcagaataatacaatcttgggagaaaatcagcagtcagcagaatcccaattcaaacaccttaggacatattctctgtgtgattgggcatattgcaaagcatcttcctaagagcacccgggacaaagtgactgatgctgtcaagtgtaagctgaatggatttcagtggtctctagaggtgatcagttcagctgttgacgccttgcagaggctttgtagagcatctgcagagacaccagcagaggagcaggaattgctgacgcaggtgtgtggggatgtactctccacctgcgagcaccgcctctccaacatcgttctcaaggagaatggaacagggaatatggacgaagacctgttggtgaagtacatttttaccttaggggatatagcccagctgtgtccagccagggtggagaagcgcatcttccttctgattcagtccgtcctggcttcgtctgctgatgctgaccactcaccatcatctcaaggcagcagtgaggccccagcgtctcagccacccccccaggtcagaggttctgtcatgccctctgtgattagagcacatgccatcattaccttaggtaagctgtgcttacagcacgaggatctggcaaagaagagcatcccagccctggtgcgagagctcgaggtgtgtgaggacgtggctgtccgcaacaacgtcatcattgtaatgtgcgatctctgcattcgctacaccatcatggtggacaagtatattcccaacatctccatgtgtctgaaggattccgacccattcatccggaagcagacactcatcttgcttaccaatctcttgcaggaggaatttgtgaaatggaagggctccctgttcttccgatttgtcagcactctgatcgattcacacccagacattgccagcttcggggagttttgcctggctcacctgttactgaagaggaaccctgtcatgttcttccaacacttcattgaatgtatttttcactttaataactatgagaagcatgagaagtacaacaagttcccccagtcagagagagagaagcggctgttttcattgaagggaaagtcaaacaaagagagacgaatgaaaatctacaaatttcttctagagcacttcacagatgaacagcgattcaacatcacttccaaaatctgccttagtattttggcgtgctttgctgatggcatcctacccctggacctggacgccagtgagttactctcagacacgtttgaggtcctcagctcaaaggagatcaagcttttggcaatgagatctaaaccagacaaagacctccttatggaagaagatgacatggccttggcaaatgtagtcatgcaggaagctcagaagaagctcatctcacaagttcagaagaggaatttcatagaaaatattattccaattatcatctccctgaagactgtgctggagaaaaataagatcccagctttgcgggaactcatgcactatctcagggaggtgatgcaggattaccgagatgagctcaaggacttctttgcagttgacaaacagctggcatcagagcttgagtatgacatgaagaagtaccaggaacagctggtccaggagcaggagctagcaaaacatgcagatgtggccgggacggctggaggtgctgaggtggcacctgtggcacaggttgccctgtgtttagaaacagtgccagttcctgctggccaagaaaaccctgccatgtcacctgccgtgagccagccctgcacacccagggcaagtgctggccatgtagcagtatcatctcctacacctgaaacagggccattgcagaggttgctgcccaaagccaggcccatgtccctgagcaccattgcaatcctgaattctgtcaagaaagccgtggagtcaaagagcaggcatcggagtcggagcttaggagtgctgcctttcactttaaattctggaagcccagaaaaaacgtgcagtcaggtgtcttcatacagtttggagcaagagtcgaatggcgagattgagcacgtgaccaagcgggccatcagcacccccgagaagagcatcagtgatgtcacgtttggagcaggggtcagttacatcgggacaccacggactccgtcgtcagccaaagagaaaattgaaggccggagtcaaggaaatgacatcttatgtttatcactgcctgataaaccgcccccacagcctcagcagtggaatgtgcggtctcccgccaggaataaagacactccagcctgcagcaggaggtccctccgaaagacccctctgaaaacagccaactaaacagcgcctcccaccagtgtccaggcaggcaggagcccttgaggaagcagtctcgtgtcctccgtgtgaaggcagctggatcacttcccgcagtccttgggcagcgctttgctgtggaacacgagagctcctcctcaggggcctggcactcaccttctattctgtatgatgtatttggttaaacactgtcaaataatagagatgtgccagatttagattttcttaccctaatctgtttaatattgtaactttattccatttgaaagtgtcaagcccattcagataagctataatctggtctttaaggaacacaactttaaaactgcagctttcttttatataaatcaagcctctgttaacttgaattccttatagtacatattttcccatctgtaatgacgaaattttgattctaatattttttctattatttataagtgcaaattttttaaaaaagtgtacagctttctaaaagtaataaaggtttagcataaatacag

SEQ ID NO: 2 - protein, 1498 aa, Homo sapiens
MVALRGLGSGLQPWCPLDLRLEWVDTVWELDFTETEPLDPSIEAEIIETGLAAFTKLYESLLPFATGEHGSMESIWTFFIENNVSHSTLVALFYHFVQIVHKKNVSVQYREYGLHAAGLYFLLLEVPGSVANQVFHPVMFDKCIQTLKKSWPQESNLNRKRKKEQPKSSQANPGRHRKRGKPPRREDIEMDEIIEEQEDENICFSARDLSQIRNAIFHLLKNFLRLLPKFSLKEKPQCVQNCIEVFVSLTNFEPVLHECHVTQARALNQAKYIPELAYYGLYLLCSPIHGEGDKVISCVFHQMLSVILMLEVGEGSHRAPLAVTSQVINCRNQAVQFISALVDELKESIFPVVRILLQHICAKVVDKSEYRTFAAQSLVQLLSKLPCGEYAMFIAWLYKYSRSSKIPHRVFTLDVVLALLELPEREVDNTLSLEHQKFLKHKFLVQEIMFDRCLDKAPTVRSKALSSFAHCLELTVTSASESILELLINSPTFSVIESHPGTLLRNSSAFSYQRQTSNRSEPSGEINIDSSGETVGSGERCVMAMLRRRIRDEKTNVRKSALQVLVSILKHCDVSGMKEDLWILQDQCRDPAVSVRKQALQSLTELLMAQPRCVQIQKAWLRGVVPVVMDCESTVQEKALEFLDQLLLQNIRHHSHFHSGDDSQVLAWALLTLLTTESQELSRYLNKAFHIWSKKEKFSPTFINNVISHTGTEHSAPAWMLLSKIAGSSPRLDYSRIIQSWEKISSQQNPNSNTLGHILCVIGHIAKHLPKSTRDKVTDAVKCKLNGFQWSLEVISSAVDALQRLCRASAETPAEEQELLTQVCGDVLSTCEHRLSNIVLKENGTGNMDEDLLVKYIFTLGDIAQLCPARVEKRIFLLIQSVLASSADADHSPSSQGSSEAPASQPPPQVRGSVMPSVIRAHAIITLGKLCLQHEDLAKKSIPALVRELEVCEDVAVRNNVIIVMCDLCIRYTIMVDKYIPNISMCLKDSDPFIRKQTLILLTNLLQEEFVKWKGSLFFRFVSTLIDSHPDIASFGEFCLAHLLLKRNPVMFFQHFIECIFHFNNYEKHEKYNKFPQSEREKRLFSLKGKSNKERRMKIYKFLLEHFTDEQRFNITSKICLSILACFADGILPLDLDASELLSDTFEVLSSKEIKLLAMRSKPDKDLLMEEDDMALANVVMQEAQKKLISQVQKRNFIENIIPIIISLKTVLEKNKIPALRELMHYLREVMQDYRDELKDFFAVDKQLASELEYDMKKYQEQLVQEQELAKHADVAGTAGGAEVAPVAQVALCLETVPVPAGQENPAMSPAVSQPCTPRASAGHVAVSSPTPETGPLQRLLPKARPMSLSTIAILNSVKKAVESKSRHRSRSLGVLPFTLNSGSPEKTCSQVSSYSLEQESNGEIEHVTKRAISTPEKSISDVTFGAGVSYIGTPRTPSSAKEKIEGRSQGNDILCLSLPDKPPPQPQQWNVRSPARNKDTPACSRRSLRKTPLKTAN

SEQ ID NO: 3 - DNA, 3070 bp, Homo sapiens
  CDS (253)...(1527)
  misc_feature (0)...(0): ARP15
agcggagttacttgggcggggccggtagcggcgggagctgcactggccagggttccggctgtatatccatgagcgccgctggcagccggggagctgcaggaaccagactgggggcgagctgagcacctgtagtcaatcacacgcagcttttaggtttgtttgaataagagatctgacctgaccggcccaactgtacaactcttcaaggaaaattcgtatttgcagtgggaagaataagtaacattgatcaagatgaatgccatgctggagactcccgaactcccagccgtgtttgatggagtgaagctggctgcagtggctgctgtgctgtacgtgatcgtccggtgtttgaacctgaagagccccacagccccacctgacctctacttccaggactcggggctctcacgctttctgctcaagtcctgtcctcttctgaccaaagaatacattccaccgttgatctgggggaaaagtggacacatccagacagccttgtatgggaagatgggaagggtgaggtcgccacatccttatgggcaccggaagttcatcactatgtctgatggagccacttctacattcgacctcttcgagcccttggctgagcactgtgttggagatgatatcaccatggtcatctgccctggaattgccaatcacagcgagaagcaatacatccgcactttcgttgactacgcccagaaaaatggctatcggtgcgccgtgctgaaccacctgggtgccctgcccaacattgaattgacctcgccacgcatgttcacctatggctgcacgtgggaatttggagccatggtgaactacatcaagaagacatatcccctgacccagctggtcgtcgtgggcttcagcctgggtggtaacattgtgtgcaaatacttgggggagactcaggcaaaccaagagaaggtcctgtgctgcgtcagcgtgtgccaggggtacagtgcactgagggcccaggaaaccttcatgcaatgggatcagtgccggcggttctacaacttcctcatggctgacaacatgaagaagatcatcctctcgcacaggcaagctctttttggagaccatgttaagaaaccccagagcctggaagacacggacttgagccggctctacacagcaacatccctgatgcagattgatgacaatgtgatgaggaagtttcacggctataactccctgaaggaatactatgaggaagaaagttgcatgcggtacctgcacaggatttatgttcctctcatgctggttaatgcagctgacgatccgttggtgcatgaaagtcttctaaccattccaaaatctctttcagagaaacgagagaacgtcatgtttgtgctgcctctgcatgggggccacttgggcttctttgagggctctgtgctgttccccgagcccctgacatggatggataagctggtggtggagtacgccaacgccatttgccaatgggagcgtaacaagttgcagtgctctgacacggagcaggtggaggccgacctggagtgaggcctccggactctggcacgctccagcagccctcctctggaagctgcgtcccctcaccccctgtttcaggtctcccatctccctcagtgacctggatctgacctcacaccatcagcagggggcacccaccatgcacacctgtctcggagtaggcagctcttcctgggagctccaggctatttttgtgcttagttactggttttctccattgcattgttaggcatggtgacaagtgacagagttcttgccctctgtccagtttcagcatctggttgcttttaagccaagtacatctagtttccctattaaaaatgtgtctgaatagcgattttgctttgccaccaaaaggcttttccctgagaacagtgaaggatgtatgtcattttgtggtggtgtatgtgtccttacatagaccttaaaaagagctcacccttccaggccaatgctgaagacacagctccgcttgggagcctgagaacccaggcttcccaggccagagtgtggcttcttaaacggcaaaggraattcctttgagtcacaagccaagttttcgccctgtctcctgagaccatttccctacgctttgctgctgctgagagttacctgaggcacttgttaaaaattcagcctcccaggtccctcccctcggagaggctgattcactgggtctgggaaggagcctggggattttaatttttcacaagtgccccagatgattctcatcaccaagcaaattttggaaatgctgttcaacagcgcccttaaattggaaacatctttgcagctcgttttattgaaattcataatcaggggtgtcctctagctcccacggtctccagagcagcaaggccggctatggagctgccgtcgtgtgaccacagtgtgatgtctcagaagggctctgggtgggctgagcatctgggctgtgcctggctctgcttttcaccctggacaaagtcgctgtggacttcaatttcttcacctctaaaatgggggacttggaccaggtagattgctgagctcactaccaggttcaaagttcaatgacaaactcagtttactgaggtttgagagaacatccctccaggggagcctgggagctgctctcccagtctaagcatgtagatatcatcgtttgccttttgtgtgtgtgtgtcccttatttgataaaaagatgttttgagttgtttttttttttaagcactcacttgtaattttagtttttaaacccaagtccctctaactttgcctttgataccaaacaattcaaaagttggatctgagtttggagaaagatatttccaacctaagtgggtattattttgaaaccagatttttaatttaatagcctatatttgtagtctgttggataggtgtttccaaagtgtgtcttctcaagtgaaaacgcaactctaggtttcaagtactccttttctccgatcctgtggtacttgaatatccaaaaaccctgcactttgaacaatcagctgttgctatctggaactaaacagaactatgagtaaaattgcctggatacttt

SEQ ID NO: 4 - protein, 425 aa, Homo sapiens
MNAMLETPELPAVFDGVKLAAVAAVLYVIVRCLNLKSPTAPPDLYFQDSGLSRFLLKSCPLLTKEYIPPLIWGKSGHIQTALYGKMGRVRSPHPYGHRKFITMSDGATSTFDLFEPLAEHCVGDDITMVICPGIANHSEKQYIRTFVDYAQKNGYRCAVLNHLGALPNIELTSPRMFTYGCTWEFGAMVNYIKKTYPLTQLVVVGFSLGGNIVCKYLGETQANQEKVLCCVSVCQGYSALRAQETFMQWDQCRRFYNFLMADNMKKIILSHRQALFGDHVKKPQSLEDTDLSRLYTATSLMQIDDNVMRKFHGYNSLKEYYEEESCMRYLHRIYVPLMLVNAADDPLVHESLLTIPKSLSEKRENVMFVLPLHGGHLGFFEGSVLFPEPLTWMDKLVVEYANAICQWERNKLQCSDTEQVEADLE

SEQ ID NO: 5 - DNA, 2161 bp, Homo sapiens
  CDS (138)...(1601)
  misc_feature (0)...(0): ARP16
ggggggccttccccgcgcagagctccgaccgcgggcggcccaggggcgggcgcgccgctgcatccccatcctcgtcgtcgcccggcacagcgcgagcgggcgagcggcgcgggcggccggagcgccgaggcccggccatggccaccaccagcaccacgggctccaccctgctgcagcccctcagcaacgccgtgcagctgcccatcgaccaggtcaactttgtagtgtgccaactctttgccttgctagcagccatttggtttcgaacttatctacattcaagcaaaactagctcttttataagacatgtagttgctacccttttgggcctttatcttgcacttttttgctttggatggtatgccttacactttcttgtacaaagtggaatttcctactgtatcatgatcatcataggagtggagaacatgcacaacccaatgatgatcattactcagaagatcactagtttggcttgcgaaattcatgatgggatgtttcggaaggatgaagaactgacttcctcacagagggatttagctgtaaggcgcatgccaagcttactggagtatttgagttacaactgtaacttcatggggatcctggcaggcccactttgctcttacaaagactacattactttcattgaaggcagatcataccatatcacacaatctggtgaaaatggaaaagaagagacacagtatgaaagaacagagccatctccaaatactgcggttgttcagaagctcttagtttgtgggctgtccttgttatttcacttgaccatctgtacaacattacctgtggagtacaacattgatgagcattttcaagctacagcttcgtggccaacaaagattatctatctgtatatctctcttttggctgccagacccaaatactattttgcatggacgctagctgatgccattaataatgctgcaggctttggtttcagagggtatgacgaaaatggagcagctcgctgggacttaatttccaatttgagaattcaacaaatagagatgtcaacaagtttcaagatgtttcttgataattggaatattcagacagctctttggctcaaaagggtgtgttatgaacgaacctccttcagtccaactatccagacgttcattctctctgccatttggcacggggtatacccaggatattatctaacgtttctaacaggggtgttaatgacattagcagcaagagctatgagaaataactttagacattatttcattgaaccttcccaactgaaattattttatgatgttataacatggatagtaactcaagtagcaataagttacacagttgtgccatttgtgcttctttctataaaaccatcactcacgttttacagctcctggtattattgcctgcacattcttggtatcttagtattattgttgttgccagtgaaaaaaactcaaagaagaaagaatacacatgaaaacattcagctctcacaatccaaaaagtttgatgaaggagaaaattctttgggacagaacagtttttctacaacaaacaatgtttgcaatcagaatcaagaaatagcctcgagacattcatcactaaagcagtgatcgggaaggctctgagggctgttttttttttttgatgttaacagaaaccaatcttagcaccttttcaaggggtttgagtttgttggaaaagcagttaactggggggaaatggacagttatagataaggaatttcctgtacaccagattggaaatggagtgaaacaagccctcccatgccatgtccccgtgggccacgccttatgtaagaatatttccatatttcagtgggcactcccaacctcagcacttgtccgtagggtcacacgcgtgccctgttgctgaatgtatgttgcgtatcccaaggcactgaagaggtggaaaaataatcgtgtcaatctggatgatagagagaaattaacttttccaaatgaatgtcttgccttaaaccctctatttcctaaaatattgttcctaaatggtattttcaagtgtaatattgtgagaacgctactgcagtagttgatgttgtgtgctgtaaaggattttaggaggaatttgaaacaggatatttaagagtgtggatatttttaaaatgcaataaacatctcagtatttg

SEQ ID NO: 6 - protein, 488 aa, Homo sapiens
MATTSTTGSTLLQPLSNAVQLPIDQVNFVVCQLFALLAAIWFRTYLHSSKTSSFIRHVVATLLGLYLALFCFGWYALHFLVQSGISYCIMIIIGVENMHNPMMIITQKITSLACEIHDGMFRKDEELTSSQRDLAVRRMPSLLEYLSYNCNFMGILAGPLCSYKDYITFIEGRSYHITQSGENGKEETQYERTEPSPNTAVVQKLLVCGLSLLFHLTICTTLPVEYNIDEHFQATASWPTKIIYLYISLLAARPKYYFAWTLADAINNAAGFGFRGYDENGAARWDLISNLRIQQIEMSTSFKMFLDNWNIQTALWLKRVCYERTSFSPTIQTFILSAIWHGVYPGYYLTFLTGVLMTLAARAMRNNFRHYFIEPSQLKLFYDVITWIVTQVAISYTVVPFVLLSIKPSLTFYSSWYYCLHILGILVLLLLPVKKTQRRKNTHENIQLSQSKKFDEGENSLGQNSFSTTNNVCNQNQEIASRHSSLKQ

SEQ ID NO: 7 - DNA, 2096 bp, Homo sapiens
  CDS (1)...(1728)
  misc_feature (0)...(0): ARP8
agcggggacctccaggattaccgctgctccagggactcagccccgagccccgtgccccatgagctggtgatcaccatcgaactgccgctgttgcgctcggccgagcaggcggcgctggaggtaacgagaaagctgctgtgcctcgactcgaggaaacctgactaccggctgcggctctcgctcccgtacccagtggacgatggccgcggcaaggcacaattcaacaaggcccggcggcagctggtggttacgctgccagtggtgctgccggccgcgcgccgggagcccgctgtcgccgtcgccgccgccgcgccggaagagtccgcggaccggtccggaactgacggccaggcctgcgcttccgctcgcgagggggaggcgggacccgcgaggagtcgcgccgaggacggaggccacgatacctgcgtggctggggctgcgggctccggggtcaccaccctgggcgacccggaggtggcgcctccgccggccgcagctggagaggagcgtgtccccaagccgggggagcaggacttgagcaggcacgcggggtcaccgccgggcagcgtggaggagccatctcctggaggagaaaactcacctggtggcggaggctccccttgtttgtcctcccggagcctggcgtggggttcttctgcgggaagagagagtgcgcgcggagatagcagtgtggaaacgcgcgaggagtcggagggcacgggcggccagcgctcagcctgcgccatgggtggtcccgggaccaagagcggggagcctttgtgtcctccgttactgtgtaatcaggacaaagaaaccttgactctgctcattcaggtgcctcggatccagccgcaaagtcttcaaggagatttgaatcccctctggtacaaattacgcttctccgcacaagacttagtttattccttctttttgcaatttgctccagagaataaattgagtaccacagaacctgtgattagcatttcttcaaacaatgcagtgatagaactggcaaaatctccagagagccatggacattggagagagtggtattatggtgtaaacaacgattctttggaggaaaggttatttgtcaatgaagaaaatgttaatgagtttcttgaagaggtcctgagctctccattcaaacagtctatgtccttgaccccaccattaattgaagttcttcaagttactgataataagattcaaattaatgcaaagttgcaagaatgtagtaactctgatcagctacaaggaaaggaggaaagagtaaatgaagaaagtcatctaactgaaaaggaatatatagaacattgtaacacccctacaactgattctgattcatctatagcagttaaagcactacaaatagatagctttggtttagttacatgctttcaacaagagtctcttgatgtttctcaaatgatacttggaaaatctcagcaacctgagtcaaaaatgcaatctgaatttataaaagaaaaaagtgctacttgttcaaatgaggaaaaagataacttaaacgagtcagtaataactgaagagaaagaaacagatggagatcacctatcttcattactgaacaaaactacggttcacaatatacctggattcgacagcataaaagaaaccaatatgcaggatggtagtgtgcaggtcattaaagatcatgtgaccaattgtgcattcagttttcagaattctttgctatatgatttggattaattctatataattttggacttttaaatattaaggttaaaaaatacctgtatctaaaattgattctgttaactgttgtcttaaaactaaaggtattaaagtataaaattaaaatttgcaattttttttaaaaaattgcaattttgattctcatgggggaaattggagataattttttttttttgcctctggagtttaaagtttccttatggagataagttttgtgattcctgtaatagatgtgtatgttttctatttgagagttaaaacatttgagagttaaaacatttagttttaatacaacctatgtatatatacttctgtgttaaattttgctttgtcattaataaaatttaaaaatattcact

SEQ ID NO: 8 - protein, 576 aa, Homo sapiens
SGDLQDYRCSRDSAPSPVPHELVITIELPLLRSAEQAALEVTRKLLCLDSRKPDYRLRLSLPYPVDDGRGKAQFNKARRQLVVTLPVVLPAARREPAVAVAAAAPEESADRSGTDGQACASAREGEAGPARSRAEDGGHDTCVAGAAGSGVTTLGDPEVAPPPAAAGEERVPKPGEQDLSRHAGSPPGSVEEPSPGGENSPGGGGSPCLSSRSLAWGSSAGRESARGDSSVETREESEGTGGQRSACAMGGPGTKSGEPLCPPLLCNQDKETLTLLIQVPRIQPQSLQGDLNPLWYKLRFSAQDLVYSFFLQFAPENKLSTTEPVISISSNNAVIELAKSPESHGHWREWYYGVNNDSLEERLFVNEENVNEFLEEVLSSPFKQSMSLTPPLIEVLQVTDNKIQINAKLQECSNSDQLQGKEERVNEESHLTEKEYIEHCNTPTTDSDSSIAVKALQIDSFGLVTCFQQESLDVSQMILGKSQQPESKMQSEFIKEKSATCSNEEKDNLNESVITEEKETDGDHLSSLLNKTTVHNIPGFDSIKETNMQDGSVQVIKDHVTNCAFSFQNSLLYDLD

SEQ ID NO: 9 - DNA, 2568 bp, Homo sapiens
  CDS (559)...(2232)
  misc_feature (0)...(0): ARP9
accgcacccgcgtcctcctcccgcgcgccggccggcagctccgggtttgccgtcgccgccgccgccactcagccgctgcacggcgcgtcctctcgggggcggcggaggcgcgtacagtcgccgccgccgccgccgccgcaccacgttccccacccggggctgcgtcaccgggagacacgttcccagccagcatgggtcggcgcccagcggcccgcccgagcactccggccgcagaaccagagtgccgccctgaggcctgctgagaacacaacaccctcccgaccgcgccaccgcgcccccctagccgggcgcgtccttgcagggcctgggctgtctccctcccactctcagaaataaggcacacgcctgggcattcgtgggccaacgggccttggctaaaccgtccccacatttgtcagcgccacagcaacatcctcagagtctgagcgaactgcgcccagcgcgggcacggagcctcccaccgccagcaacctgcggccccggagaaggcagcgagcgcagtgacagcgcctcaccgccaccagctcctggaccaccatggccaagaaccgcagggacagaaacagttggggtggattttcggaaaagacatatgaatggagctcagaagaggaggagccagtgaaaaaggcaggaccagtccaagtcctcattgtcaaagatgaccattcctttgagttagatgaaactgcattaaatcggatccttctctcggaggctgtcagagacaaggaggttgttgctgtatctgttgctggagcatttagaaaaggaaaatcattcctgatggacttcatgttgagatacatgtacaaccaggaatcagttgattgggttggagactacaatgaaccattgactggtttttcatggagaggtggatctgaacgagagaccacaggaattcagatatggagtgaaatcttccttatcaataaacctgatggtaaaaaggttgcagtgttattgatggatactcagggaacctttgatagtcagtcaactttgagagattcagccacagtatttgcccttagcacaatgatcagctcaatacaggtatataacttatcccaaaatgtccaggaggatgatcttcagcacctccagcttttcactgagtatggcagactggcaatggaggaaacattcctgaagccatttcagagtctgatatttcttgttcgagactggagtttcccatacgaattttcatatggagccgatggtggtgccaaattcttggaaaaacgcctcaaggtctcagggaaccagcatgaagaactacagaacgtcagaaaacacatccattcctgtttcaccaacatttcctgttttctgctacctcatcctggcttaaaagtagctaccaatccaaactttgatggaaaattgaaagaaatagatgatgaattcatcaaaaacttgaaaatactgattccttggctacttagtcccgagagcctagatattaaagagatcaatgggaataaaatcacctgccggggtctggtggagtacttcaaggcttatataaagatctatcaaggtgaagaattaccacatcccaaatccatgttacaggccacagcagaagctaacaatttagcagccgtggcaactgccaaggacacatacaacaaaaaaatggaagagatttgtggtggtgacaaaccatttctggccccaaatgacttgcagaccaaacacctgcaacttaaggaagaatctgtgaagctattccgaggggtgaagaagatgggtggggaagaatttagccggcgttacctgcagcagttggagagtgaaatagatgaactttacatccaatatatcaagcacaatgatagcaaaaatatcttccatgcagctcgtaccccagccacactgtttgtagtcatctttatcacatatgtgattgctggtgtgactggattcattggtttggacatcatagctagcctatgcaatatgataatgggactgacccttatcaccctgtgcacttgggcatatatccggtactctggagaataccgagagctgggagctgtaatagaccaggtggctgcagctctgtgggaccagggaagtacaaatgaggctttgtacaagctttacagtgcagcagcaacccacagacatctgtatcatcaagctttccctacaccaaagtcggaatctactgaacaatcagaaaagaaaaaaatgtaatgcaaattttaagaaatacaggtgcatgaccaattgtcaattaaatattcagttttatgtctccatgcaaacattcaaagtgcttccatcagaacggagtaaaatactaaacacctctgaagactgcaaactggattagttcttttacttcagtgtttaataagcagatgtatgtatgcatggttatactattttgttaacatgtacaatttcctgatttttcttcaaaaatgctgttataaagtatttgtctatttatgataacagtacacgtgttctgcttgaatttactaaattctactactgggttataattaaatcatgtgatattcc

SEQ ID NO: 10 - protein, 558 aa, Homo sapiens
MAKNRRDRNSWGGFSEKTYEWSSEEEEPVKKAGPVQVLIVKDDHSFELDETALNRILLSEAVRDKEVVAVSVAGAFRKGKSFLMDFMLRYMYNQESVDWVGDYNEPLTGFSWRGGSERETTGIQIWSEIFLINKPDGKKVAVLLMDTQGTFDSQSTLRDSATVFALSTMISSIQVYNLSQNVQEDDLQHLQLFTEYGRLAMEETFLKPFQSLIFLVRDWSFPYEFSYGADGGAKFLEKRLKVSGNQHEELQNVRKHIHSCFTNISCFLLPHPGLKVATNPNFDGKLKEIDDEFIKNLKILIPWLLSPESLDIKEINGNKITCRGLVEYFKAYIKIYQGEELPHPKSMLQATAEANNLAAVATAKDTYNKKMEEICGGDKPFLAPNDLQTKHLQLKEESVKLFRGVKKMGGEEFSRRYLQQLESEIDELYIQYIKHNDSKNIFHAARTPATLFVVIFITYVIAGVTGFIGLDIIASLCNMIMGLTLITLCTWAYIRYSGEYRELGAVIDQVAAALWDQGSTNEALYKLYSAAATHRHLYHQAFPTPKSESTEQSEKKKM

SEQ ID NO: 11 - DNA, 2920 bp, Homo sapiens
  CDS (141)...(1022)
  misc_feature (0)...(0): ARP13
tacgcacactatagggaatttggccctcgaggcaagaattcggcacgaggcggcggggtccgtggccagagctgcagagagacaaggcggcggcggctgctgtgctgggtgcagtgaggaagaggccctcggtggtgcccatggctggccaggatcctgcgctgagcacgagtcacccgttctacgacgtggccagacatggcattctgcaggtggcaggggatgaccgctttggaagacgtgttgtcacgttcagctgctgccggatgccaccctcccacgagctggaccaccagcggctgctggagtatttgaagtacacactggaccaatacgttgagaacgattataccatcgtctatttccactacgggctgaacagccggaacaagccttccctgggctggctccagagcgcatacaaggagttcgataggaagtacaagaagaacttgaaggccctctacgtggtgcaccccaccagcttcatcaaggtcctgtggaacatcttgaagcccctcatcagtcacaagtttgggaagaaagtcatctatttcaactacctgagtgagctccacgaacaccttaaatacgaccagctggtcatccctcccgaagttttgcggtacgatgagaagctccagagcctgcacgagggccggacgccgcctcccaccaagacaccaccgccgcggcccccgctgcccacacagcagtttggcgtcagtctgcaatacctcaaagacaaaaatcaaggcgaactcatcccccctgtgctgaggttcacagtgacgtacctgagagagaaaggcctgcgcaccgagggcctgttccggagatccgccagcgtgcagaccgtccgcgagatccagaggctctacaaccaagggaagcccgtgaactttgacgactacggggacattcacatccctgccgtgatcctgaagaccttcctgcgagagctgccccagccgcttctgaccttccaggcctacgagcagattctcgggatcacctgtgcgtagctgccctggcgcaggggtggggggcttggtcctcagatgctgtcccccagctactggcccagggtcaggctctggggtggccgaggtgacgtgtacccaccctcctcctgttgccatctggcactgcagggcaagagagggggttgttggggctgcccccaccatgcacagccagtccacactgtccaaaggcagaggaggtggggtcggtcagggttgtccaggccggtctctcaggcacagctgggccaggaggcagggtgatggggggctctctagatttggggctcatcatcctggtcaaagtcctgcctgtaaccgctcccaggccccagaccctcagctctctcatctagaaagggctctcggcatttctccccagatcattgtaggatgccaagcatatatcacaggaactgagcagctatagtgtctgatcctacaggcgctttgtgatgggtcaaatgctcagtgtggctgagcacagatgactcgtaaaaaactccgtaacagcatctcaaaacactggtgaatgctaaggaaagtgatgcctcgaaagaaccataaaacccctcaggccttcatctaagtcatcgaacaccgtcttttcaaatgggactacctccagcgccctcctttcttcttggagcactaacagtcgaaggggggagctgattttggtgtgaattctgaagcagctcggttaagatatcgtaaagacaaatcttgaatcttaaaatcaatgtttctaccccactgtgtattcaagaatcacatttgcccaaagggcagcctggcctttgtcctggcccctggggggtgacttctgaaccttgcactttcccaaggggcaggagttacctttgtcactcatcaggggccctgatggtttatggtaactcatggttatggtgtgactcaagataggggtgtaggggtggccattccagaaagaccaattacatggttagagggtgcgggctttgagtgatgtgagaccagcctgacctctggggagggcatggggctggagactgatttcagtctcaaggcgtcctgtcaaggtcgctggaccccaaactcccctcaaaggcaaatttacacccatttactcagcagtgctcctaagcgcctgccaaatgcaacgccctttctgagatggggaaggtctctcctacaccctggaagaccatgaaagatgttgagggctgactgactttggctagtggatgggaagcctgggagagttcagagccaggctgagcctcatggagacaaatttgatcatgcgcatagrggtacctatctggtggagacagaaggaggcactacattcagcagcatcctaccagccggggccagagcaggcaagatggacaatgctccatcacctgaatgcgccaggctgtgtcctagagagaagcttccagaagcctcccaacttcattagcccctgcagagggatgatgactccagcctctgtaggttcctgcttgagaaaactcaatgctgccaggcgaacttactgtttgttccagccaaaacctggtgacagggagataggccctggaacccctctttgagcagcagttcctttagaaagcttgcaattgtgggccaggcatggtggctcrcrcctgtaatcccagcactttgrgaggctgagttgggtggatcacttgtcataggagttcargaccagcctggtcaacatggcaaaaccctgtctctactaaaatacaaaaattagccaagcgtggtggtgcacgcctgtrrtcccagctactcrggaggctgaggcrggagaatgacttgaacccaggaggcagaggttgtagtgagccrggatcgtgccattgcactgcagcctrggtgacagaacaagactccatctc

SEQ ID NO: 12 - protein, 294 aa, Homo sapiens
MAGQDPALSTSHPFYDVARHGILQVAGDDRFGRRVVTFSCCRMPPSHELDHQRLLEYLKYTLDQYVENDYTIVYFHYGLNSRNKPSLGWLQSAYKEFDRKYKKNLKALYVVHPTSFIKVLWNILKPLISHKFGKKVIYFNYLSELHEHLKYDQLVIPPEVLRYDEKLQSLHEGRTPPPTKTPPPRPPLPTQQFGVSLQYLKDKNQGELIPPVLRFTVTYLREKGLRTEGLFRRSASVQTVREIQRLYNQGKPVNFDDYGDIHIPAVILKTFLRELPQPLLTFQAYEQILGITCA

SEQ ID NO: 13 - DNA, 1095 bp, Homo sapiens
  CDS (113)...(661)
  misc_feature (0)...(0): ARP20
agaggatcccaatttagctgcgcacagrgaggtgattttctgagtgtgactcctctgttcctggcaccctgtgcatccttagccatagcttacaagagaacagctggttgtgatggcaggaggccctcccaacaccaaggcggagatggaaatgtccctggcagaagaactgaatcatggacgccaaggggaaaaccaagagcacctggtgatagcagaaatgatggagcttggatctcggtcccggggtgcctcccagaagaagcagaagttggaacaaaaagctgctggctctgcttcagccaaacgagtttggaatatgactgccacccgacccaagaaaatggggtcccagctgccaaagcccagaatgctgagagaatcaggccatggggatgcccatctccaggagtacgctggcaatttccaaggcatacgtttccattatgatcgcaacccagggacagatgcagtggcgcagactagcctggaagagttcaatgtactggagatggaagtcatgagaagacagctgtatgcagtcaaccggcgtctgcgcgccctggaggaacagggcgccacctggcgccacagggagaccctgatcatcgccgtgctggtgtcggccagcattgccaacctgtggctgtggatgaaccagtgatcgccccagcgcggcctccgtattggagccctccctgcttccccttctttctttcctctttccccaggccgccactgcccttgcccctttcatctcccagcagccctcaggagcgtcaggatcattttcaactctggttaggcctcctacctggggaggccaggtcactgcactgggaggtcctggctgctgcgaagctggaggaggactgcgtgggctgagatgccaccctttgaagggtgaacagcatggcggcatctgggccccacagtaacacctagtggcaaccttgccttcctgacctcagcggcccttctgttccatcctctgtgggcaggggtgtggctttgttttcctccctcgtttgcttccacctcgtgcacagcgctctgcacagacaacacgctcaataaaagttcagccatagcagc

SEQ ID NO: 14 - protein, 183 aa, Homo sapiens
MAGGPPNTKAEMEMSLAEELNHGRQGENQEHLVIAEMMELGSRSRGASQKKQKLEQKAAGSASAKRVWNMTATRPKKMGSQLPKPRMLRESGHGDAHLQEYAGNFQGIRFHYDRNPGTDAVAQTSLEEFNVLEMEVMRRQLYAVNRRLRALEEQGATWRHRETLIIAVLVSASIANLWLWMNQ

SEQ ID NO: 15 - DNA, 3007 bp, Homo sapiens
  CDS (38)...(1378)
  misc_feature (0)...(0): ARP24
gattccattactggttgaactatggcacaaggataaaatgagtaaagatttacttctgggaattgcgagaatccagctttctaacatcttgtcttcagaaaaaactcgttttttaggttctaatggtgaacagtgttggcgtcaaacttacagtgaaagtgtgcctgttatagcagcacaaggatcaaataacaggatagcagatctttcttacacagtgactctagaagattatggactagtaaaaatgcgtgagatttttatctctgattcatctcagggtgtatctgccgtacagcaaaagccgtcttctcttcctccagcaccttgtccttcagagatccagacagagcctcgtgaaacgttagaatacaaagcagcacttgagctagaaatgtggaaggagatgcaagaagatatatttgaaaatcagctgaagcagaaagaactggctcatatgcaggctcttgcagaggaatggaagaaaagggaccgagaaagagaatcactagtaaagaaaaaggtggctgaatatactattctagaaggaaaacttcaaaaaactctaattgacttggagaagcgagagcagcagcttgctagtgtggaatcagagcttcaaagagaaaaaaaggaactgcaatcagaacgtcagcggaacctgcaagaactgcaggactctatccgtagggccaaagaggactgtattcaccaagtagaactagaaaggttaaaaatcaaacagctcgaagaggataaacaccgmcttcagcaacagcttaatgatgctgaaaataagtataagatttkgraaaaagagttccaacagttcaaggaccagcaaaacaacawaccagaaatccgtctacagtctgaaataaatcttctcaccttggaaaaggttgaacttgaaagaaagttggaatctgcaactaagtctaaactgcattacaagcagcagtggggacgagctttgaaagaacttgccagacttaaacagagggagcaagaaagtcaaatggctcgtcttaaaaaacagcaggaagaattggaacagatgagactacgttaccttgccgctgaggaaaaagatacagtaaaaaccgagcgacaagaattgttggatataagaaatgaattgaacaggttaaggcaacaagagcaaaaacaataccaggattccacagagattgcaagtggaaaaaaggatggcccccatggcagtgtattggaagaaggtttggatgattatttgactcgcctgatagaagaaagggatactttgatgagaacgggtgtgtataatcacgaggatcgaataataagtgaactcgaccgacagatcagagagattttggcaaaaagcaatgccagtaattaataacatttggaaaagctttatagagactctaagtctaaattttaatttctttgtaaaaacctcaaaagtgaggaaaatggatgttttaaaatggtattttcaattttttataagcaaaattttgtatgttattgtatagtatttatttgatcttatttactttatgctacctctcccacactggttttatttgtaatttgcatttatatactcattttaaatgacttttcagtgtttttcatagtttataatctgatggctaactaactttcaaaacagcttttaactaagtttgttgtaggagaaatgactgcggtaatttccagttctataatgtttcatgttgagccaaaagagtatatcttgcactttaaaaacactgcgtcctattccatttgaatgtaaattcttaaaagttgagaccaattgtaaccagtttaactcattttagatgacctttttttcttataatatttgcaagtgacaagtttgaaaacaaagcaagatcagtgcagagaagccacatgacatgttgggtgacagattggtcatttatttaaataaagttaatacagattaaatttgtttcaagagctattgaattttcaaattttcagtgtatttataacttttaagaacatgaagtattagcttaatatagttttctctgttggtttcttcctcaagtttgcattgttttcttttgttaaaattagagatttctttttattttccagttatagtaatactagctgtcagcttaaaccctctgtaatagaacatggaaacagacacataaagacattagctgaaaaaatagagtgaaaatcaactattttttactcccgctaatttcaatcaatcgttttcaaaagcgcacgagattcactcattggatttatgcagtgccctgtctgtataaaaactcttaagagttcctttatatcatattcttcagagccaacatttgtcctcaaagcaacgtttcccacctccttttactgagtactgaaaaagttttagcaaagtcacagattaggttgaatttcaaaatatatgtactttaaaaagttctgatttccaaatataataaagttaaaattaagtatatactttaggaagttaccaataacttccatcaaagcagagtaggatatatggtaacattaattttcggtcatttcaaatgaggtatatttcttattagggaaattaaagtccctatatttattatatattttttccttattaacagagtattaatctagtaaaaaataacccagcagtaaataataaagaaattactgaatgagaggataatgaatctgaatcatagcaggaatttgcaatattaattatggttagctatttttctctcattgatttttgtgccacttgaatggaacagaagcaagccttatgttttggaaggtctgcggtaaaatgctgtgactgtttactttcaattgcattgtgtgttgcctgtgactgctttcaaacgctagagggggcctctgatttaaagaaataaaaaggacttttctaaaatggatgtgtagtttattttgccttttgtaaagctcttttggctattgtaacttaacaaataaaatcataattgtgtgc

SEQ ID NO: 16 - protein, 447 aa, Homo sapiens
  VARIANT 243, 258, 259, 272: Xaa = Any Amino Acid
MSKDLLLGIARIQLSNILSSEKTRFLGSNGEQCWRQTYSESVPVIAAQGSNNRIADLSYTVTLEDYGLVKMREIFISDSSQGVSAVQQKPSSLPPAPCPSEIQTEPRETLEYKAALELEMWKEMQEDIFENQLKQKELAHMQALAEEWKKRDRERESLVKKKVAEYTILEGKLQKTLIDLEKREQQLASVESELQREKKELQSERQRNLQELQDSIRRAKEDCIHQVELERLKIKQLEEDKHXLQQQLNDAENKYKIXXKEFQQFKDQQNNXPEIRLQSEINLLTLEKVELERKLESATKSKLHYKQQWGRALKELARLKQREQESQMARLKKQQEELEQMRLRYLAAEEKDTVKTERQELLDIRNELNRLRQQEQKQYQDSTEIASGKKDGPHGSVLEEGLDDYLTRLIEERDTLMRTGVYNHEDRIISELDRQIREILAKSNASN

SEQ ID NO: 17 - DNA, 3937 bp, Homo sapiens
  CDS (240)...(1013)
  misc_feature (0)...(0): ARP26
cgaccgctgtcctccaacagcgcagggcagagcggctggcgccgccggagcgcggagccacgaccctccctggccgcctttgtctactggccgtgcggcccggaaccgccactctccagggccggggacgcgcccgcagctgtcggtgacagctcctccctaccgcaaccctccggggcggaggggcggtcgggccgggccctgctagcccgcgaccgcaagcccgcgctcgcggatcgatgcccccgcagcagggggaccccgcgttccccgaccgctgcgaggcgcctccggtgccgccgcgtcgggagcgcggtggacgcgggggacgcgggcctggggagccggggggccgggggcgtgcggggggtgccgaggggcgcggcgtcaagtgcgtgctggtcggcgacggcgcggtgggcaagacgagcctggtggtgagctacaccaccaacggctaccccaccgagtacatccctactgccttcgacaacttctccgcggtggtgtctgtggatgggcggcccgtgagactccaactctgtgacactgccggacaggatgaatttgacaagctgaggcctctctgctacaccaacacagacatcttcctgctctgcttcagtgtcgtgagcccctcatccttccagaacgtcagtgagaaatgggtgccggagattcgatgccactgtcccaaagcccccatcatcctagttggaacgcagtcggatctcagagaagatgtcaaagtcctcattgagttggacaaatgcaaagaaaagccagtgcctgaagaggcggctaagctgtgcgccgaggaaatcaaagccgcctcctacatcgagtgttcagccttgactcaaaaaaacctcaaagaggtctttgatgcagccatcgtcgctggcattcaatactcggacactcagcaacagccaaagaagtctaaaagcaggactccagataaaatgaaaaacctctccaagtcctggtggaagaagtactgctgtttcgtatgatgctggcaagacacccagaaaggctattttcagatgaaatcgatattagaagctatattagctgaaacaactccttttactgcgtagaacctatatcgagagtgtgtgtatatgtattataggaggagctctcaattttatgtattctttctgcctttaattttcttgtttgtttgagcttagggatgagatacttatgcaagatatttttgaagtaaattaaacatttttcacatctctggaaatttagagttctagacctctggttaatttatatctaatatgaagaagacacctctaatctggatgttaagaatgaagttctgctacattataatgtacagaagagcaaaagggaggaacactatggttaaccctctcttgatgaagggctacttaatgcacagtgcattatgtacacaggtcaaccatggtaacaatagttcttagctttgaaactccatgcaaaccatgcctttttttaaggagcaaaaatctgagaaaaaaagtgagagacctctgcctacaaaacctcaaaccagtcacttttgtcaattgctaatacccagttacttatgatttaaaaacaaccaacagaaaacatcccactgactgtatggcactctatagtcaaaaaaggaaacttccttattgggacttttctttcttagtccagttgtgttgacacatatgaacacagacaaagtgctatgcggaggaaagcaagtgttggtcagtagtttcatgttttagggagtggttcctgtggagatcagaaagtgacatttgctttcggtactgtaatacgtgcaccaaactgcctcaatcctaggtaacgagggcaacagggagcacctgtctggattgtttttaaacctccatactcaagctgtctcttcggcagggaggtgaatactcttgaaaggccaacagcaagtgtttgtgggacacaacacagataattttttcttaagtcggccaagatgtacttctctgtgtgcacacccatgcacactcatgcacacagatacataggtctgtatggctgtatttgctgttgattcagactttcacaccattaatggggaaaagcgtggccacaaaaacagatgctaggaagcttggcttcctcttcttgttgacccttttttgaaccaacatcttttttattatattcagagtatgtttttaagtgtatcttaatatatacattttttaggacatcttaaatctaaacaaaaaataaaatgaacatctcttgaaacctgttaaaacaaccagttaaagccacagatggctttcagggcagtagcagcagaggccagtggactctgaggactcctgaggggcggggcgtgtagccagccaggtgcatgccgggaccatggcccccatacttggctgcttcctgtgacagtgaaatacatccttcaaggtggcagctgttagggctgaatcttctggagaaaaaggtgccatctcaggagaatagcttttactctggtaggaatgcttccgagacaccacaaggcagcctgaacactcagttgcagggtcgggcttgcggtgggtgacccagagccaccaaagtcacatccacaactaatgagggaaatctgtaaagccagttagatagaagaattttatttttctgtgggttttgtgttgtcttttttatgttaaaaagaaatccagtttgtgtttttctataggaaaagtaaaagatcaggttatactttaggttaggggttctatttattcctgttagtaaataaaattaacaaatttctttgtttaacaaaagattaatctttaaaccactaaaatacatagactgattgattattcaacacattggaattgatgtcggtcatagtttcctgaagcatttagttacaacctgaaggaataaaatgatttgtggaaatgcttaaaatagacctaactgaatacagtctcatcttgccgcgcctggcttacctatctgtggaaagctaggcttcccaggctgggctctgcctgtctggtgcctggaggtgtgggagggaagatgagttatttaactggtaagcgatttgaaacactatttttatattaaagtaaatggcatggagtatagtgcaaattcatttttaagatagaacacaaaacttgaaagaagttttatgcgtgtgacagtgtatggggctgcagttggtctccctggaggggacttccacacctcctgcctttaggccatgggtggaaagtgctcagtgaagtacacctgtgtggcccagttctgaaagctttatacagttgaattttaagtggggttgataacaccttggactgttagtgttaaaaatctagtgggttgacctttaaatgcaacagtttttaaaatatattgctgcattttatagaatagtaaaggtacgattatacttgagattttcctccatttttatttcttcgtgaacatagagtttggggccgaaaatgtttttaaagtatgtgtttgagttaaatataaagttggttcacttcaaagctaaaaaattgttaaacttgcagcttggtattgcagagaagattttataagaattttgctttagagaatgccactttggctgaactacaagtgtaggccaccattataatttataaatacagcatacttcaaaactgtttgttatctcttgttaccatgtatgtataaatggaccttttataaccttgttctctgcttgacagactcaagagaaactacccaggtattacacaagccaaaatgggagcaaggccttctctccagactatcgtaacctggtgccttaccaagttgtgcttttctgttttcaagtgtaaatgatgttgagcagaatgttgtacttgaaaatgctataagtgagatggtatgaaataaattctgacttatg

SEQ ID NO: 18 - protein, 258 aa, Homo sapiens
MPPQQGDPAFPDRCEAPPVPPRRERGGRGGRGPGEPGGRGRAGGAEGRGVKCVLVGDGAVGKTSLVVSYTTNGYPTEYIPTAFDNFSAVVSVDGRPVRLQLCDTAGQDEFDKLRPLCYTNTDIFLLCFSVVSPSSFQNVSEKWVPEIRCHCPKAPIILVGTQSDLREDVKVLIELDKCKEKPVPEEAAKLCAEEIKAASYIECSALTQKNLKEVFDAAIVAGIQYSDTQQQPKKSKSRTPDKMKNLSKSWWKKYCCFV

SEQ ID NO: 19 - DNA, 1401 bp, Homo sapiens
  CDS (45)...(1085)
  misc_feature (0)...(0): ARP28
ccccctcccctcctgcagcctcctgcgccccgccgagctggcggatggagctgcgcagcgggagcgtgggcagccaggcggtggcgcggaggatggatggggacagccgagatggcggcggcggcaaggacgccaccgggtcggaggactacgagaacctgccgactagcgcctccgtgtccacccacatgacagcaggagcgatggccgggatcctggagcactcggtcatgtacccggtggactcggtgaagacacgaatgcagagtttgagtccagatcccaaagcccagtacacaagtatctacggagccctcaagaaaatcatgcggaccgaaggcttctggaggcccttgcgaggcgtcaacgtcatgatcatgggtgcagggccggcccatgccatgtattttgcctgctatgaaaacatgaaaaggactttaaatgacgttttccaccaccaaggaaacagccacctagccaacgggatagctgggagtatggccaccctgctccacgatgcggtaatgaatccagcagaagtggtgaagcagcgcttgcagatgtacaactcgcagcaccggtcagcaatcagctgcatccggacggtgtggaggaccgaggggttgggggccttctaccggagctacaccacgcagctgaccatgaacatccccttccagtccatccacttcatcacctatgagttcctgcaggagcaggtcaacccccaccggacctacaacccgcagtcccacatcatctcaggcgggctggccggggccctcgccgcggccgccacgacccccctggacgtctgtaagacccttctgaacactcaggagaacgtggccctctcgctggccaacatcagcggccggctgtcgggtatggccaatgccttccggacggtgtaccagctcaacggcctgccggctacttcaaaggcatccaggcgcgtgtcatctaccagatgccctccaccgccatttcttggtctgtctatgagttcttcaagtactttctcaccaagcgccagctggaaaatcgagctccatactaaaggaagggatcatagaatcttttcttaaagtcattctctgcctgcatccagccccttgccctctcctcacacgtagatcatttttttttttgcagggtgctgcctatgggccctctgctccccaatgccttagagagaggaggggacggcacggccgctcaccggaaggctgtgtgcggggacatccgaggtggtggtggacaggaaggacttgggaaggggagcgagaaattgctttttctcttcctccctgggcagaatgtagcttttctgcttcactgtggcagcctcctccctggatccttagatcccagaggagggaagaaaatttgcagtgactg

SEQ ID NO: 20 - protein, 347 aa, Homo sapiens
MELRSGSVGSQAVARRMDGDSRDGGGGKDATGSEDYENLPTSASVSTHMTAGAMAGILEHSVMYPVDSVKTRMQSLSPDPKAQYTSIYGALKKIMRTEGFWRPLRGVNVMIMGAGPAHAMYFACYENMKRTLNDVFHHQGNSHLANGIAGSMATLLHDAVMNPAEVVKQRLQMYNSQHRSAISCIRTVWRTEGLGAFYRSYTTQLTMNIPFQSIHFITYEFLQEQVNPHRTYNPQSHIISGGLAGALAAAATTPLDVCKTLLNTQENVALSLANISGRLSGMANAFRTVYQLNGLPATSKASRRVSSTRCPPPPFLGLSMSSSSTFSPSASWKIELHTKGRDHRIFS

SEQ ID NO: 21 - DNA, 3318 bp, Homo sapiens
  CDS (252)...(2054)
  misc_feature (0)...(0): ARP30
gtgggggccaggcagcacagatgaagcatttacctatctaggtaagtcaggaggagctcaaaaggagaagaaaacagtaggaggcagggcacgagggcctctgtctccatctctgccctttgaaacaaaagggtatttcttttctctcttcagcccccaacccagtggaggcccggcttgggacattgttcacttcccctcgcttcccctctagaagccccctttgccatccctgcaccttgtttcgggtgatgcccgagagggagctgtggccagcggggactggctcagaacccgtgacccgtgtcggcagctgtgacagcatgatgagcagcacctccacccgctctggatctagtgatagcagctacgacttcctgtccactgaagagaaggagtgtctgctcttcctggaggagaccattggctcactggacacggaggctgacagcggactgtccactgacgagtctgagccagccacaactcccagaggtttccgagcactgcccataacccaacccactccccggggaggtccagaggagaccatcactcagcaaggacgaacgccaaggacagtaactgagtccagctcatcccaccctcctgagccccagggcctaggcctcaggtctggctcctacagcctccctaggaatatccacattgccagaagccagaacttcaggaaaagcaccacccaggctagcagtcacaaccctggagaaccggggaggcttgcgccagagcctgagaaagaacaggtcagccagagcagccaacccaggcaggcacctgccagcccccaggaggctgcccttgacttggacgtggtgctcatccctccgccagaagctttccgggacacccagccagagcagtgtagggaagccagcctgcccgaggggccaggacagcagggccacacaccccagctccacacaccatccagctcccaggaaagagagcagactccttcagaagccatgtcccaaaaagccaaggaaacagtctcaaccaggtacacacaaccccagcctcctcctgcagggttgcctcagaatgcaagagctgaagatgctcccctctcatcaggggaggacccaaacagccgactagctcccctcacaacccctaagccccggaagctgccacctaatattgttctgaagagcagccgaagcagtttccacagtgacccccagcactggctgtcccgccacactgaggctgcccctggagattctggcctgatctcctgttcactgcaagagcagagaaaagcacgtaaagaagctctagagaagctggggctaccccaggatcaagatgagcctggactccacttaagtaagcccaccagctccatcagacccaaggagacacgggcccagcatctgtccccagctccaggtctggctcagcctgcagctccagcccaggcctcagcagctattcctgctgctgggaaggctctggctcaagctccggctccagctccaggtccagctcagggacctttgccaatgaagtctccagctccaggcaatgttgcagctagcaaatctatgccaattcctatccctaaggccccaagggcaaacagtgccctgactccaccgaagccagagtcagggctgactctccaggagagcaacacccctggcctgagacagatgaacttcaagtccaacactctggagcgctcaggcgtgggactgagcagctacctttcaactgagaaagatgccagccccaaaaccagcacttctctgggaaagggctccttcttggacaagatctcgcccagtgtcttacgtaattctcggccccgcccggcctccctgggcacggggaaagattttgcaggtatccaggtaggcaagctggctgacctggagcaggagcagagctccaagcgcctgtcctaccaaggacagagccgtgacaagcttcctcgccccccctgtgtcagtgtcaagatctccccaaagggtgtccccaatgaacacagaagggaggccctgaagaagctgggactgttgaaggagtagactctgcgaccagtacagaccctgtcctggctgaacaagaagagacacatgctccacttgggagcctttgccaccacgcaactcagggctcaagatgaatgggagggagagatttgagtccaagcatacatttatattcagtgttgtgccattgagttcccatgtggatcattctgaaggtgatctccacaagagggtgtgtgtgtgtgtgtttggtgtgtgtgtggagggggggccgctggatacatcactgaagctattgatataacacaatgagtcactgttcagaaaaaaaaaaaaaaaaaaaaatttcttacattgggtagagtccagcctagtgagagctgagtgaaggggctggccatgcctgagacaaaaagtcaaatgagacaatggacgtgtcaatgacttgaaaaaaagtcacatccagcaaatgcagggtcacatgaaatatgggcctcctggaatccctacagtggatggagactggctcataccttgccagatccctctctcagttccagccttctggacaaggcctgggctaagaggagctgattcgttatctcttcacccactgccctctcagtatcaccagtctcaaagacaggatacgtccctgtaatgcaatctctcggttgattgatagcagaacagctcttgttggtctgagaaggcagcataagtgtccacatatttatgccgctccctccaccaggtagagtccttctccacaggcttgataaattcaatcaccaactgtgctgtcgtccctgactctgctactcccgttcttcctgctttcctgctccgtatctcagtctgcactgaccccagcgctgggctgacatcaagatgggagccccagccacgggctttataaacacccaagaaccgtttcagatcttctctgtgctgatgcaggtagttttaaatttttctcagttccagtgatagaaaacccacacaatacatcctctgccagtcttaatagaatatcagaggtaagaggggcctcagagaagctctgacgcagtgctgctggggaagggaagtgactaaccccgggtcagcctgccatttagggaaagagctgaggttcttacccttgttgcatgctgccacctctccttagccagtgctcttgtacatccacacagcaccctaaggagccatagtcaccatcaaagactcaaccctaaggcccttcaagatctcaaagtgccttctgaagcatcagagattaaatattgttcaaact

SEQ ID NO: 22 - protein, 601 aa, Homo sapiens
MPERELWPAGTGSEPVTRVGSCDSMMSSTSTRSGSSDSSYDFLSTEEKECLLFLEETIGSLDTEADSGLSTDESEPATTPRGFRALPITQPTPRGGPEETITQQGRTPRTVTESSSSHPPEPQGLGLRSGSYSLPRNIHIARSQNFRKSTTQASSHNPGEPGRLAPEPEKEQVSQSSQPRQAPASPQEAALDLDVVLIPPPEAFRDTQPEQCREASLPEGPGQQGHTPQLHTPSSSQEREQTPSEAMSQKAKETVSTRYTQPQPPPAGLPQNARAEDAPLSSGEDPNSRLAPLTTPKPRKLPPNIVLKSSRSSFHSDPQHWLSRHTEAAPGDSGLISCSLQEQRKARKEALEKLGLPQDQDEPGLHLSKPTSSIRPKETRAQHLSPAPGLAQPAAPAQASAAIPAAGKALAQAPAPAPGPAQGPLPMKSPAPGNVAASKSMPIPIPKAPRANSALTPPKPESGLTLQESNTPGLRQMNFKSNTLERSGVGLSSYLSTEKDASPKTSTSLGKGSFLDKISPSVLRNSRPRPASLGTGKDFAGIQVGKLADLEQEQSSKRLSYQGQSRDKLPRPPCVSVKISPKGVPNEHRREALKKLGLLKE

SEQ ID NO: 23 - DNA, 1690 bp, Homo sapiens
  CDS (98)...(1313)
  misc_feature (0)...(0): ARP33
ggcacgagcacggcagccctacactcggcctggaagaattgtttttcttctctggaaaggtgaacatttatagcatttatttcccaaatctgttaacatggcaaaatatgtcagtctcactgaagctaacgaagaactcaaggtcttaatggacgagaaccagaccagccgccccgtggccgttcacacctccaccgtgaacccgctcgggaagcagctcttgccgaaaacctttggacagtccagtgtcaacattgaccagcaagtggtaattgggtatgcctcagagaccagcagcatcaaacatccctgtggtaggaagcccaaacccacccagcactcactttgcctctcagaaccagcattcctactcctcacctccttgggccggcagcacaacaggaaaggagagaagaatggcatgggcctgtgccgtctttccatgaaggtctgggagacggtgcagaggaaagggaccacttcctgccaggaagtggtgggcgagctggtcgccaagttcagagctgccagcaaccacgcctcaccaaacgagtcagcttatgacgtgaaaaacataaaacggcgcacctacgatgccttaaacgtgctgatggccatgaatatcatctccagggagaaaaagaagatcaagtggattggtctgaccaccaactcggctcagaactgtcagaacttacgggtggaaagacagaagagacttgaaagaataaagcagaaacagtctgaacttcaacaacttattctacagcaaattgctttcaagaacctggtgctgagaaaccagtatgtggaggagcaggtcagccagcggccgctgcccaactcagtcatccacgtgcccttcatcatcatcagcagtagcaagaagaccgtcatcaactgcagcatctccgacgacaaatcagaatatctgtttaagtttaacagctcctttgaaatccacgatgacacagaagtgctgatgtggatgggcatgacttttgggctagagtccgggagctgctctgccgaagaccttaaaatggccagaaatttggtcccaaaggctctggagccgtacgtgacagaaatggctcagggaacttttggaggtgtgttcacgacggcaggttccaggtctaatggcacgtggctttctgccagtgacctgaccaacattgcgattgggatgctggccacaagctccggtggatctcagtacagtggctccagggtggagaccccagcagtcgaggaggaagaggaggaggacaacaacgatgacgacctcagtgagaatgacgaggatgactgacgtcctctcgccttaagattcagcttcaggaaaacatttagggaaaagaaacttttttttttttttttaatgtgaggttttctgtttcttttttgcctactccccaagaagatattggtaagctatagaatttagatatgcacctctgataagcaaggattgtttcccgtatgattaagacgtgctgttgatgtgtgttttgataccagtgtgctgacacagaatctttatttactttttaggattttgtgttttcattttctatttttctttaaatgcagagttcattgttgccccttaacagtttttcctgagtttactgaagaaattgtacttcatccacatccatgaaaataaaatgctctccttttgtgc

SEQ ID NO: 24 - protein, 405 aa, Homo sapiens
MAKYVSLTEANEELKVLMDENQTSRPVAVHTSTVNPLGKQLLPKTFGQSSVNIDQQVVIGYASETSSIKHPCGRKPKPTQHSLCLSEPAFLLLTSLGRQHNRKGEKNGMGLCRLSMKVWETVQRKGTTSCQEVVGELVAKFRAASNHASPNESAYDVKNIKRRTYDALNVLMAMNIISREKKKIKWIGLTTNSAQNCQNLRVERQKRLERIKQKQSELQQLILQQIAFKNLVLRNQYVEEQVSQRPLPNSVIHVPFIIISSSKKTVINCSISDDKSEYLFKFNSSFEIHDDTEVLMWMGMTFGLESGSCSAEDLKMARNLVPKALEPYVTEMAQGTFGGVFTTAGSRSNGTWLSASDLTNIAIGMLATSSGGSQYSGSRVETPAVEEEEEEDNNDDDLSENDEDD

SEQ ID NO: 25 - DNA, 504 bp, Homo sapiens
  misc_feature (0)...(0): ARP6
tgcacataacttttcttcttgagcaagtgagtatgagcaagattggataagctaaaaataacttcaaatgttgttaattttgctcatttggtatactaacatcactttacagacttgtaaaatattagagataatatccaatgttggcaagaaagagtaaacaagtatgttggtgggaatataaaatgatacagccttttgagaagataatttgctatgatctagaaaaatatttagtatgcatcccatttgacccagaaattccacactgaagtccatattctacagagatatgaacccacgtgtaattatatgattgtatatatttacatataagtgcaaaatattttgaggtagcattgtttggaatagcatgatactggggctgggtccagaggctcacacctataatcccagcactttgggaggctaaggggggaggatggcttgaggccagcacagttcgggaccagactgggaaacatactgagactccatctctac

SEQ ID NO: 26 - DNA, 2189 bp, Homo sapiens
  misc_feature (0)...(0): ARP10
taattgtttataagagaggaaccactggaaagagtttaaaatccttgttccattttcaggttgggaagtgtacgtgtgtgggggtgggtattaaacaagacttgagcattaagcatttcccctcctgagcccagctcctctcctcacttgttgactaagccctgcttcactgagcattttctttttttctgcccacaacctgagtgccccatggaggtgaaggacatagcctgagtctgtgccatcagtcagtagaacaaatgggcttgctctgcctaacatcgggccatctcggattccacctcttttataggaatgagtgatgaagagaggtgggttccgctttgccgctgtattaatacacatcaggggccagctcctagcactaaatcacgctacggcatacatctgttatcggactcttcaccagtgtgatgaacaagactgcagatagaggcttccttgtgtcattctttataggattttcctaaaagaataaatagctcagatctctgccaacactctcccctgtcactgtgatgaattcagcttctttcttaaacatgcagccacaaatcttttccctttttcccctcccccagtggtagaaagttttgagttgcaattgactgaatttaaagatattaataaggctagagaggtgaagagcaggaagaaacagcatgtcttccttgcctttttgtactaatagtaacaaaaggccaaggaaaaagactgtaaatgaccatagggttgttaaatagacccatcttgaaagtgtagcacctttattttctttgtatcttcattctccctcctttacggctttctagcatgcaggtgtctagtgcacagaaccacaccctggatcttagtgagcaagcatgctaacctgcttctcagacttgatacaccagaacagggatttccctgcaagtgtctccctcatgctgaagtgaactagctgaatatgctcttaaagaaagtagtcagaaaagaaaaaaaagattatcttctccaaaatttgagaccaagtagctgtaaaaaacataataaacccagatgaaaaccaggcttcgttttcttgaaatgattttcttcatcagaatggtagatcagagccattggtgtgcagattccaatcctttaaaaagtaaacacatgccttttgataaagcggaattgaggtgatcagaaattctgttgagaacccagctatttgtgtgagtatattttagctatcccaaaaactttttctgacctttctctttctgggataggatatgtgtgcttagagtatcattcagaagggtacctaatagttaatctgttaattagttacatcaggtttcaaatactaggtcagtgatatgagagcgagagagagagatttgaattgtcaaatgtattgtcagatgcattcacaagagcaggactgctttatctgttttgttcactactgtacccctagcatctaaatgaatacctagcccatagaataaacccactggttatttgttgtaagaataaattaatagaatcttaaagttgaaacagagtgattcctaatatattgtaaccacatggttggattcagtagttccattttaggatgtcccttttctcaggagtcatagggcaaattcttattgcccactgtgtctttttaaagtttaaatgttttttaaaattaaacatgtttcttagtaaatattgaaggggtataaaagaacatttatagcagatatgtaaggagtaaataaaatacagcaaatatccatgtacctaccattcactttaagaaaaagttaaaattttcttttataaaaggtttaaaagttttaaaaagttaaaatctttaaaaatcttctccctgctctcctcagaggtaaaagttattctgattgttttttatcgtttgtcattcttttgtttttttcaagtagttttagcagacatatgtatcttcaagcaatacattgtccagttttgtgtttttgaaccttgtgtatatgaaatcaaagtatgtcatattttatgttgcagctttcaccgggttctgtttttgagattcaattatgctttgtcctgccaaaatctatgtttcaactgccatgtatgtggtattctattgtatgaatatgattaatatgttataattaaatgttcttgatgaacatttggattgt

SEQ ID NO: 27 - DNA, 2576 bp, Homo sapiens
  misc_feature (0)...(0): ARP12
cttggacgagggaaagatgcaatatgtcacttacacatttcttcaagctgaaaagtgcttgtggtgaacctgcttacacaaattatgttggtggctttcatggatgtctagattacattttcattgacttaaatgctttagaggttgaacaggtgattccattacctagtcatgaagaagttaccacccaccaggccttacctagtgtttcccatccctctgatcacatagcacttgtatgtgatttaaaatggaaatagatgtgtgtttaatggaattgaagtctgaaaaggaagtagttattttagcagaaaatttaatatgaatcaaagcttatatgtaaacttcaaggaggaatggtaaaatgttcagccctcctagttatgttcctgatgtcttcgttatgaaactgttgatgtttgcatcatacatcttctctttccttgttttcctctacaattggaggagaaacaaatatatttcttactagcaaaatagaaaattgaattatttttctccaaattgagactctcagaaaaggaagattgaattagcgtgttttttgtttgtttgtttttgtttttgtttttgtttttttgagatggagtttcactcttgttgcccagagctgagattgcgccactgcactccagcctcactgcaacctccgccccctgggtttaagcgattctcctgcctcagcttcccgagtagctgggattacaggcatgcgccaacatgtctggctaattttgtatttttagtagaaatggggtttcgccacgttggccaggctggtcttgaactcctgacctcaggtgatccacccacctcggcctcccaaagtgttgggattacaggcgtgagccaccgcacccggcccttgtgtacatttttataagagaatttttttagctaggagttcagaatttttaaagtaccatttgaatgatcttaatttttctttcatgacaacacattccaaaatgaatcatgcttatgtactaagagggaaaatgtatttaagttaagggtgagagacttaagttataggtgaccttagagacctaaggtgagagacttgacacatggaaggagtaacattagggtctacctctacctcaatttagttagcgatttactacaatttcagagctttaacaaaagataaaaataaatcgtcaccaattgttattgcttctcatctttcatttttcaatgaacaagtaaggtattttcattcttatttttaggattttagtttttagtgtatggtacaaatgaacacagtttatattctaattcttactgcagctcattttaatttttaggatgcaagcacaatttagtattcaaagtgagtagcaacatattcaacttgatcccattgtcttcagttactcttgcccatgaaaaatgttcataaatgaacagggtatttgaccatatgatattagaaaatacagcacattactttatgagaaactacctactgatatgggcttgaaattttggatgaatcattgagcatttctacactagaagtaatttcaaaattgttggtttttataaacaggaaaaaggttgagtagtgggacttttaagcatctctgaaataaaaaacttctttttacagaccaagcattatagttttgagttacagacaacagtgtgtatatatgtaatatatatatagtaaaatgaaatttaaatatgaagccaaactttttaaaattagaaactacaaatggttatactgattagtgtctagcctagagtggtaaccatgctttactaattcagttatgaaatacattatttataatgcattagctgtattagctgttgcttttttgatgttcaggataactatgttatctcatttctgcatttaattaatagctcgagtattaaaagcccactcccttcaagaaaagctttgattttccccagtcatgaaagcccttgtttcaaattctttaatctctgaacctagtatcataagaatttcctcttttgataacatctgtactttcatattctgctcactatcaaatgtattgttaacacttagtaagtttgaaaatgaaggggttttatctgcatttgacattgaaccttgaagtactttaagtactccaaggggaaaattaaagtggaagtttcttcggatcttgtttagaaaaaactataaataaaaaattgatgctaccaaattgtgccttcctaaataacatttttgagagcattttaacagcagtttacaaatatgtaaataatagattaaaaccaaatcttgattctcttgtgaatttttttttcattttaaaaatatgttttgggctgttttcaaagaaagatgttgatagaacccttagagtgactgtgggagaaaacaaagtgtcacatcaacaaagtttgagaaacattttgacagacaaaattcgaacatgccatgaaaaaagcatacagcttccacattaacactgggctaggattaaactctagtcaggaaaaactcaggcactttaacaggacattccatgtcctattatcctttaattttggatgttttc

SEQ ID NO: 28 - DNA, 521 bp, Homo sapiens
  misc_feature (0)...(0): ARP18
cagagattatgtgggcttttccatgggaagaaccactctacgttatcaccttctacatagtagcagttgaagccaaatggacagaaagcccgagacaacatgaagttgttctacaagttattttggagaaattgacttaccataccactcatcaacccatgcaaaagcctgtctatgtccaatcagcagaatgtctcggaccacctaaaaagtaaaagaaggagactgaaataatagcatctttgatgaaaactatctggaagacaagttgttaacaattctggggatcttggtgattacagagttcttaatccctctgtccataggtgatgacaattacaggctgcctataggtcctatagtgctcacacacctccagcccttccccatggtgtacacacacttgcagtatattcatctctttgtcttatttgagagtagggctgggtgtgtgtacaaactaatgacaaatacttgacagtcacacagcagtgatacaaataaatatctaggttaattac

SEQ ID NO: 29 - DNA, 644 bp, Homo sapiens
  misc_feature 591: n = A,T,C or G
ggcacgaggaaagaacgcacactttcaattttattgaggccttcaacactatttaaaagaaaatgtaagaatttgacattctggagttattataacattagaaaatgagcataacattcactctgattttagccattaagggagattagtaaacagactgctacagtgttccatagttggactgtgcatccaaaacatttttttatctttaataaatggtacagtttttatgtagttttcgaatgtaagaagaaaggaatgctgaccaaaacttgatttcatcagcttcatgaaaaggactagtgtcattaacctgttgaacagaattggtttattaaaaaaatcatttccagtagtgtgaaacctttacgagtctttaacatctaaatgttatgactccttgtaccttaagttttccagtctttcttatttatatcatctccaagtacctctggctcctttcctcttgctcaccggarccttagttttcctcaacagaatgctttgttaaagtagcccacagttgcaggatccatagcaccgtcgtgcagactagcagcccaaaggtgtgtttggtttggcttatacggngttttgctttttaaactacttgccataatttaaaagtggcaacacctagactt

SEQ ID NO: 30 - DNA, 1460 bp, Homo sapiens
  misc_feature (0)...(0): ARP21
atttaatcgactcactatagggaatttggaggaccggccttgcgagcggcgacactataaaatggcgcgtgctgcaacccgcgccgcttcggagagagaaatgctggggtgcagcttcaagcttaggaccacccaccatgcctatccaggtgctgaagggcctgaccatcactcattaagaacagaggaggctgcctgttactcctggtgttgcatccctccagacactctgctgtttcctgcctaggcgtggctgcagcatggctaggaaagcgctgccacccacccacctgggccagagctggttctgctcctgctgcagggacactgagctggctatctcggcgcttcgggcaagaactgcaacaggctctcctgggtcctgcaggtgtacagccgggcccctgccttgtgcctcagctctcgagagctgctgctgccgggtgacctgatccaacctgataaggtgccatcttcagctaccactgcaaggccctgagggcaacagcagcacggcactgccacccggctgctgatggcctggtgccagctgggagtcctcccggcacttcgaggccactgagccacccttccagccccagcccaccatggacaggggtatccagcttcctcctcaacctcgtcctctgcccctgagccagtgacgcccaaggacatgcctgttacccaggtcctgtaccagcactactgtcaagggcatgacagtgctggaggccgtcttggagatccaggccatcactggcagcaggctgtctccatggtgccagggcccgccagcaccaggctcatgctgggacccaacccagtgcacaaggacttggctgctgagccacacacccaggagaaggtggataagtgggctaccaagggcttcctgcaggctaggggaggagccacccccgcttccctattgtgaccaggcctatggggaggagctgtccatacgccaccgtgagacctgggcctggctctcaaggacagacaccgcctggcctggtgctccaggggtgaagcaggccagaatcctgggggagctgctcctggtttgagctgcattcaggaagtgcgggacatggtaggggaggcaaaaagccttgggcactaccctccctgtggagctgttcggtgtccgtcgagctagccacaccctgacaccatgttcaagggtaccggaagagaagggtgtctgcccccaacctcccctgtgggtgtcactggccagatgtcatgagggaagcaggccttgtgagtggacactgaccatgagtccctggggggagtgatcccccaggcatcgtgtgccatgttgcacttctgcccaggcagcagggtgggtgggtaccatgggtgcccacccctccaccacatggggccccaaagcactgcaggccaagcagggcaaccccacacccttgacataaaagcatcttgaagctttt

SEQ ID NO: 31 - DNA, 774 bp, Homo sapiens
  misc_feature (0)...(0): ARP22
ttgtaaaacgacggccgtgccaagctaaaattaaccctcactaaagggaataagcttgcggccgcttaattaattaattatagagacagggtctcactatgttggccaggttggtcttgaactcttggcctccagtaatcctcccaccttggtttcccaaagtgctaggattacaggtatgagccactgtgcctggtcatgcctggtattttgatggaatgcaggatactatgtttcaaatagtatagaggctaagtttcttctggcaggtaactagaataagggctgatcacctcattcaatcaaagttttgaagttagatttaaagctgataacttctggtcaacctttatttgtaggacataattcttttgaggtctcaaccgaaggcctcgaatatttccctttgtcattcccatttggctggccttgaaatctaatttttttgtctacctactcctgtaagaaacaaaaaackgtgccttaaagtttttaaagtctttgggtaatagctctcctactgaatttctccttctgaatttgaaaaatacctaacagcaaaagctaagtcaaatgttgagtttactaccctctttctctttattttcccaaagagatggggtctcactatgttgtcccaggctggagtgcaagkggtgtgatcaatgctcactgtaacctcgaactgctgggctyaagcaatcctcctgcctyagcctcctgagtagctgggactacagacaggkgscaccatgctggctgagttcawtactc

SEQ ID NO: 32 - DNA, 386 bp, Homo sapiens
  misc_feature (0)...(0): ARP29
tgtgggcggctacagggagaaattcaagaggaagttcttggtggtgccctccatgagtacaaagaagcctcagtccccaggacacccttccgtgcatggtgtcactgacatctttatttcttttgtcacgttctgtaaatcacaatgaatggggtattcttcttctattawawatttgttaagtcttttttggcatctttaaaaaaaagtggtaactttatcctatgtaatatccctgttaagtcctaaaagtcttttctgatgtctattttgtctgaaatttgcacagctactatagctttatttcggttcatattttcataatccatgttttctcatccttttatatttgtgaatgtgtaaacgtaactttcttgtgcatagct

SEQ ID NO: 33 - DNA, 3091 bp, Homo sapiens
  CDS (790)...(1809)
gagcgacgcccacggcctgtctcggccaccagcgtgttccagcgagcgcccagccacctcgctcgcagcctccccagcgcagcagcccggctgtgggcctgcggcagccgggtcttcctggtccccacctcctggggccgacgggcggcaggaaggggctcggcgggacgcgccgtcagggacctgaggaggaacaacggaacgcgttcggaacggcctggactcccgagactcacccgactcgtggccacaccgggagaactgaagcggcagtagccggcggagacgcccgacccgaaggccggctgctagggagcagacagctgaaccgcttgccagacgccgaaacccagtgacgccctccaccgctccaccgtgctcccggctccccgcccccgccgcccgcgggccccaaggcgcatgcgccgcctgtcctggaggggcccatttccgtccgtcgtggggggaggcacagtgagtccactggggcacggcagcgtctaagccacaagccgagcacataagccaggtcctaacggagcctatgtgtaagtccactactggtgcaaggttgcacacttctaagaagagcggcgtggggggctcggcgaccttcgcttcagtcgctcccccgtgcagtcccctgtgcccaagacacagcctgatgcttgtgctccggtgggcggacttggaggcggcgggaactgcaattggtggctttgaaggcgcggcgagcgggaacagctcttgaggagtgagactgcaggagatgtgggccgtgccaaagagatggatgagactgttgctgagttcatcaagaggaccatcttgaaaatccccatgaatgaactgacaacaatcctgaaggcctgggattttttgtctgaaaatcaactgcagactgtaaatttccgacagagaaaggaatctgtagttcagcacttgatccatctgtgtgaggaaaagcgtgcaagtatcagtgatgctgccctgttagacatcatttatatgcaatttcatcagcaccagaaagtttgggatgtttttcagatgagtaaaggaccaggtgaagatgttgacctttttgatatgaaacaatttaaaaattcgttcaagaaaattcttcagagagcattaaaaaatgtgacagtcagcttcagagaaactgaggagaatgcagtctggattcgaattgcctggggaacacagtacacaaagccaaaccagtacaaacctacctacgtggtgtactactcccagactccgtacgccttcacgtcctcctccatgctgaggcgcaatacaccgcttctgggtcaggagttgacaattgctagcaaacaccatcagattgtgaaaatggacctgagaagtcggtatctggactctcttaaggctattgtttttaaacagtataatcagacctttgaaactcacaactctacgacacctctacaggaaagaagccttggactagatataaatatggattcaaggatcattcatgaaaacatagtagaaaaagagagagtccaacgaataactcaagaaacatttggagattatcctcaaccacaactagaatttgcacaatataagcttgaaacgaaattcaaaagtggtttaaatgggagcatcttggctgagaggaaagaacccctccgatgcctaataaagttctctagcccacatcttctggaagcattgaaatccttagcaccagcgggtattgcagatgctccactttctccactgctcacttgcatacccaacaagagaatgaattattttaaaattagagataaataagacgtgcgtggtttcttaagcacagctcctccttcttgatattgcacatgcacttcagttcatggctagctgtatagcttccgtctgtaaacttgtattttcaagaatccttggtattgaatttttagaaatgctcacataattgttgggactgattcattcctccacgatatgcctcctctctctgatatcctgctaactgtagccgttgtggcatttgagatgacaggacatatatatatatggccccacacttgaccttgagtgcctgaatgctctgaaatcaagcatatggcacagcgctcaagacttttgggtttgtgtccttttttctatggctgtctcttctcaattctggagaggtctggttccagtggctggtttccagggattgattcttaagctctggatcacagagagaagcaacaaggaactatactcaactcaaaactttttaggagaatcatgaaattggtctattcaaaggatggagttgagtccattctgttattgttgcaagaggttgcatatttggtgagtcagttatataaaatagtgttcttattgtaaatatgatacttctcataatctattttatcatgtgtataacattcaaactgacaaatatattgacttatgaataaaggtgtcaaaaaactggcacatcagttaattttgatcaaagtacttcagtgatcatcactaaataccctatctttttaaaaattttttcctttctaattttttatttctttatttatttattgagacggggtctcgctgtgtcactccagcctrggtgacagagtgagactccrtcttaaaaaataaataaataaaataaaataaatgacatcactttggttcagagctctaaaatggagggaggaagccattctaaaaaggactccctacatgacctgcaacttgaaaaaaaattaaaagctccaaaaaaaaaaacaatacaggagcttaccttgaacctttgaattgggccaaattgcgatgaccactgcatcctggaaaattttatttcaccagcactacaactcctcaacagcaccaaccaataaactatggatttttgtactaagccagttgcctctttcaaaacaacttgtcaacttgtctaatcaccctcagctttttttaaaaacccctcctctaccctctctcttcagaacacaagtggcttctagctgaatctgtctcccaaattgcaattcctaagacctcaataaaaacaccttgtcttgctaaaaaaaaaaaaaaaaaaaaaaaa

SEQ ID NO: 34 - protein, 339 aa, Homo sapiens
MDETVAEFIKRTILKIPMNELTTILKAWDFLSENQLQTVNFRQRKESVVQHLIHLCEEKRASISDAALLDIIYMQFHQHQKVWDVFQMSKGPGEDVDLFDMKQFKNSFKKILQRALKNVTVSFRETEENAVWIRIAWGTQYTKPNQYKPTYVVYYSQTPYAFTSSSMLRRNTPLLGQELTIASKHHQIVKMDLRSRYLDSLKAIVFKQYNQTFETHNSTTPLQERSLGLDINMDSRIIHENIVEKERVQRITQETFGDYPQPQLEFAQYKLETKFKSGLNGSILAERKEPLRCLIKFSSPHLLEALKSLAPAGIADAPLSPLLTCIPNKRMNYFKIRDK